SEQUENCE LISTING (44 sequences)

SEQ ID NO: 1 - DNA, 1494 bp, Homo sapiens
atgcccacccccgacgccaccacgccacaggccaagggcttccgcagggccgtgtctgagctggacgccaagcaggcagaggccatcatgtccccgcggttcattgggcgcaggcagagcctcatcgaggacgcccgcaaggagcgggaggcggcggtggcagcagcggccgctgcagtcccctcggagcccggggaccccctggaggctgtggcctttgaggagaaggaggggaaggccgtgctaaacctgctcttctccccgagggccaccaagccctcggcgctgtcccgagctgtgaaggtgtttgagacgtttgaagccaaaatccaccatctagagacccggcccgcccagaggccgcgagctgggggcccccacctggagtacttcgtgcgcctcgaggtgcgccgaggggacctggccgccctgctcagtggtgtgcgccaggtgtcagaggacgtgcgcagccccgcggggcccaaggtcccctggttcccaagaaaagtgtcagagctggacaagtgtcatcacctggtcaccaagttcgaccctgacctggacttggaccacccgggcttctcggaccaggtgtaccgccagcgcaggaagctgattgctgagatcgccttccagtacaggcacggcgacccgattccccgtgtggagtacaccgccgaggagattgccacctggaaggaggtctacaccacgctgaagggcctctacgccacgcacgcctgcggggagcacctggaggcctttgctttgctggagcgcttcagcggctaccgggaagacaatatcccccagctggaggacgtctcccgcttcctgaaggagcgcacgggcttccagctgcggcctgtggccggcctgctgtccgcccgggacttcctggccagcctggccttccgcgtgttccagtgcacccagtatatccgccacgcgtcctcgcccatgcactcccctgagccggactgctgccacgagctgctggggcacgtgcccatgctggccgaccgcaccttcgcgcagttctcgcaggacattggcctggcgtccctgggggcctcggatgaggaaattgagaagctgtccacgctgtactggttcacggtggagttcgggctgtgtaagcagaacggggaggtgaaggcctatggtgccgggctgctgtcctcctacggggagctcctgcactgcctgtctgaggagcctgagattcgggccttcgaccctgaggctgcggccgtgcagccctaccaagaccagacgtaccagtcagtctacttcgtgtctgagagcttcagtgacgccaaggacaagctcaggagctatgcctcacgcatccagcgccccttctccgtgaagttcgacccgtacacgctggccatcgacgtgctggacagcccccaggccgtgcggcgctccctggagggtgtccaggatgagctggacacccttgcccatgcgctgagtgccattggctag

SEQ ID NO: 2 - DNA, 1029 bp, Homo sapiens
atgagccccgcggggcccaaggtcccctggttcccaagaaaagtgtcagagctggacaagtgtcatcacctggtcaccaagttcgaccctgacctggacttggaccacccgggcttctcggaccaggtgtaccgccagcgcaggaagctgattgctgagatcgccttccagtacaggcacggcgacccgattccccgtgtggagtacaccgccgaggagattgccacctggaaggaggtctacaccacgctgaagggcctctacgccacgcacgcctgcggggagcacctggaggcctttgctttgctggagcgcttcagcggctaccgggaagacaatatcccccagctggaggacgtctcccgcttcctgaaggagcgcacgggcttccagctgcggcctgtggccggcctgctgtccgcccgggacttcctggccagcctggccttccgcgtgttccagtgcacccagtatatccgccacgcgtcctcgcccatgcactcccctgagccggactgctgccacgagctgctggggcacgtgcccatgctggccgaccgcaccttcgcgcagttctcgcaggacattggcctggcgtccctgggggcctcggatgaggaaattgagaagctgtccacgctgtactggttcacggtggagttcgggctgtgtaagcagaacggggaggtgaaggcctatggtgccgggctgctgtcctcctacggggagctcctgcactgcctgtctgaggagcctgagattcgggccttcgaccctgaggctgcggccgtgcagccctaccaagaccagacgtaccagtcagtctacttcgtgtctgagagcttcagtgacgccaaggacaagctcaggagctatgcctcacgcatccagcgccccttctccgtgaagttcgacccgtacacgctggccatcgacgtgctggacagcccccaggccgtgcggcgctccctggagggtgtccaggatgagctggacacccttgcccatgcgctgagtgccattggctag

SEQ ID NO: 3 - DNA, 792 bp, Mus musculus
ggtggttttcctttgaaaaacacgatgataatatggccacaaccgcggccgtagatcccgggaccatggagaagccgcggggagtcaggtgcaccaatgggttctccgagcgggagctgccgcggcccggggccagcccgcctgccgagaagtcccggccgcccgaggccaagggcgcacagccggccgacgcctggaaggcagggcggcaccgcagcgaggaggaaaaccaggtgaacctccccaaactggcggctgcttactcgtccattctgctctcgctgggcgaggacccccagcggcaggggctgctcaagacgccctggagggcggccaccgccatgcagtacttcaccaagggataccaggagaccatctcagatgtcctgaatgatgctatatttgatgaagatcatgacgagatggtgattgtgaaggacatagatatgttctccatgtgtgagcatcaccttgttccatttgtaggaagggtccatattggctatcttcctaacaagcaagtccttggtctcagtaaacttgccaggattgtagaaatctacagtagacgactacaagttcaagagcgcctcaccaaacagattgcggtggccatcacagaagccttgcagcctgctggcgttggagtagtgattgaagcgacacacatgtgcatggtaatgcgaggcgtgcagaaaatgaacagcaagactgtcactagcaccatgctgggcgtgttccgggaagaccccaagactcgggaggagttcctcacactaatcaggagctgag

SEQ ID NO: 4 - DNA, 753 bp, Homo sapiens
atggagaagggccctgtgcgggcaccggcggagaagccgcggggcgccaggtgcagcaatgggttccccgagcgggatccgccgcggcccgggcccagcaggccggcggagaagcccccgcggcccgaggccaagagcgcgcagcccgcggacggctggaagggcgagcggccccgcagcgaggaggataacgagctgaacctccctaacctggcagccgcctactcgtccatcctgagctcgctgggcgagaacccccagcggcaagggctgctcaagacgccctggagggcggcctcggccatgcagttcttcaccaagggctaccaggagaccatctcagatgtcctaaacgatgctatatttgatgaagatcatgatgagatggtgattgtgaaggacatagacatgttttccatgtgtgagcatcacttggttccatttgttggaaaggtccatattggttatcttcctaacaagcaagtccttggcctcagcaaacttgcgaggattgtagaaatctatagtagaagactacaagttcaggagcgccttacaaaacaaattgctgtagcaatcacggaagccttgcggcctgctggagtcggggtagtggttgaagcaacacacatgtgtatggtaatgcgaggtgtacagaaaatgaacagcaaaactgtgaccagcacaatgttgggtgtgttccgggaggatccaaagactcgggaagagttcctgactctcattaggagctga

SEQ ID NO: 5 - DNA, 469 bp, Homo sapiens
ctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 6 - DNA, 461 bp, Foot-and-mouth disease virus
agcaggtttccccaactgacacaaaacgtgcaacttgaaactccgcctggtctttccaggtctagaggggtaacactttgtactgcgtttggctccacgctcgatccactggcgagtgttagtaacagcactgttgcttcgtagcggagcatgacggccgtgggaactcctccttggtaacaaggacccacggggccaaaagccacgcccacacgggcccgtcatgtgtgcaaccccagcacggcgactttactgcgaaacccactttaaagtgacattgaaactggtacccacacactggtgacaggctaaggatgcccttcaggtaccccgaggtaacacgcgacactcgggatctgagaaggggactggggcttctataaaagcgctcggtttaaaaagcttctatgcctgaataggtgaccggaggtcggcacctttcctttgcaattactgaccac

SEQ ID NO: 7 - DNA, 551 bp, Encephalomyocarditis virus
cgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctcccctcaagcgtattcaacaaggggctgaaggatgcccagaaggtaccccattgtatgggatctgatctggggcctcggtgcacatgcttttcatgtgtttagtcgaggttaaaaaacgtctaggccccccgaaccacggggacgtggttttcctttgaaaaacacgatgataata

SEQ ID NO: 8 - DNA, 84 bp, Artificial Sequence
  Viral 2A peptide spacer sequence
cgcgcgaaacgcgcgccggtgaaacagaccctgaactttgatctgctgaaactggcgggcgatgtggaaagcaacccgggcccg

SEQ ID NO: 9 - DNA, 45 bp, Artificial Sequence
  Flexible linker sequence
ggaggtggcgggtccgggggcgggggtagcggtggcgggggctcc

SEQ ID NO: 10 - DNA, 592 bp, Woodchuck hepatitis B virus
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctg

SEQ ID NO: 11 - DNA, 247 bp, Woodchuck hepatitis B virus
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgt

SEQ ID NO: 12 - DNA, 224 bp, Simian virus 40
agcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggta

SEQ ID NO: 13 - DNA, 6633 bp, Artificial Sequence
  Recombinant vector
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccggtcgcgtactagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcagatctttgtcgatcctaccatccactcgacacacccgccagcggccgctgccaagcttccgagctctcgaattcaaaggaggtacccaccatggccaccatgagccccgcggggcccaaggtcccctggttcccaagaaaagtgtcagagctggacaagtgtcatcacctggtcaccaagttcgaccctgacctggacttggaccacccgggcttctcggaccaggtgtaccgccagcgcaggaagctgattgctgagatcgccttccagtacaggcacggcgacccgattccccgtgtggagtacaccgccgaggagattgccacctggaaggaggtctacaccacgctgaagggcctctacgccacgcacgcctgcggggagcacctggaggcctttgctttgctggagcgcttcagcggctaccgggaagacaatatcccccagctggaggacgtctcccgcttcctgaaggagcgcacgggcttccagctgcggcctgtggccggcctgctgtccgcccgggacttcctggccagcctggccttccgcgtgttccagtgcacccagtatatccgccacgcgtcctcgcccatgcactcccctgagccggactgctgccacgagctgctggggcacgtgcccatgctggccgaccgcaccttcgcgcagttctcgcaggacattggcctggcgtccctgggggcctcggatgaggaaattgagaagctgtccacgctgtactggttcacggtggagttcgggctgtgtaagcagaacggggaggtgaaggcctatggtgccgggctgctgtcctcctacggggagctcctgcactgcctgtctgaggagcctgagattcgggccttcgaccctgaggctgcggccgtgcagccctaccaagaccagacgtaccagtcagtctacttcgtgtctgagagcttcagtgacgccaaggacaagctcaggagctatgcctcacgcatccagcgccccttctccgtgaagttcgacccgtacacgctggccatcgacgtgctggacagcccccaggccgtgcggcgctccctggagggtgtccaggatgagctggacacccttgcccatgcgctgagtgccattggctaaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctcccctcaagcgtattcaacaaggggctgaaggatgcccagaaggtaccccattgtatgggatctgatctggggcctcggtgcacatgcttttcatgtgtttagtcgaggttaaaaaacgtctaggccccccgaaccacggggacgtggttttcctttgaaaaacacgatgataatagccaccatggagaagggccctgtgcgggcaccggcggagaagccgcggggcgccaggtgcagcaatgggttccccgagcgggatccgccgcggcccgggcccagcaggccggcggagaagcccccgcggcccgaggccaagagcgcgcagcccgcggacggctggaagggcgagcggccccgcagcgaggaggataacgagctgaacctccctaacctggcagccgcctactcgtccatcctgagctcgctgggcgagaacccccagcggcaagggctgctcaagacgccctggagggcggcctcggccatgcagttcttcaccaagggctaccaggagaccatctcagatgtcctaaacgatgctatatttgatgaagatcatgatgagatggtgattgtgaaggacatagacatgttttccatgtgtgagcatcacttggttccatttgttggaaaggtccatattggttatcttcctaacaagcaagtccttggcctcagcaaacttgcgaggattgtagaaatctatagtagaagactacaagttcaggagcgccttacaaaacaaattgctgtagcaatcacggaagccttgcggcctgctggagtcggggtagtggttgaagcaacacacatgtgtatggtaatgcgaggtgtacagaaaatgaacagcaaaactgtgaccagcacaatgttgggtgtgttccgggaggatccaaagactcgggaagagttcctgactctcattaggagctgagccacctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccctggctgactgatacaatcgatttctggatccgcaggcctctgctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaagcttaacgcggtaaccacgtgcggacccaacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 14 - DNA, 6542 bp, Artificial Sequence
  Recombinant vector
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccggtcgcgtactagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcagatctttgtcgatcctaccatccactcgacacacccgccagcggccgctgccaagcttccgagctctcgaattcaaaggaggtacccaccatggccaccatgagccccgcggggcccaaggtcccctggttcccaagaaaagtgtcagagctggacaagtgtcatcacctggtcaccaagttcgaccctgacctggacttggaccacccgggcttctcggaccaggtgtaccgccagcgcaggaagctgattgctgagatcgccttccagtacaggcacggcgacccgattccccgtgtggagtacaccgccgaggagattgccacctggaaggaggtctacaccacgctgaagggcctctacgccacgcacgcctgcggggagcacctggaggcctttgctttgctggagcgcttcagcggctaccgggaagacaatatcccccagctggaggacgtctcccgcttcctgaaggagcgcacgggcttccagctgcggcctgtggccggcctgctgtccgcccgggacttcctggccagcctggccttccgcgtgttccagtgcacccagtatatccgccacgcgtcctcgcccatgcactcccctgagccggactgctgccacgagctgctggggcacgtgcccatgctggccgaccgcaccttcgcgcagttctcgcaggacattggcctggcgtccctgggggcctcggatgaggaaattgagaagctgtccacgctgtactggttcacggtggagttcgggctgtgtaagcagaacggggaggtgaaggcctatggtgccgggctgctgtcctcctacggggagctcctgcactgcctgtctgaggagcctgagattcgggccttcgaccctgaggctgcggccgtgcagccctaccaagaccagacgtaccagtcagtctacttcgtgtctgagagcttcagtgacgccaaggacaagctcaggagctatgcctcacgcatccagcgccccttctccgtgaagttcgacccgtacacgctggccatcgacgtgctggacagcccccaggccgtgcggcgctccctggagggtgtccaggatgagctggacacccttgcccatgcgctgagtgccattggctaaagcaggtttccccaactgacacaaaacgtgcaacttgaaactccgcctggtctttccaggtctagaggggtaacactttgtactgcgtttggctccacgctcgatccactggcgagtgttagtaacagcactgttgcttcgtagcggagcatgacggccgtgggaactcctccttggtaacaaggacccacggggccaaaagccacgcccacacgggcccgtcatgtgtgcaaccccagcacggcgactttactgcgaaacccactttaaagtgacattgaaactggtacccacacactggtgacaggctaaggatgcccttcaggtaccccgaggtaacacgcgacactcgggatctgagaaggggactggggcttctataaaagcgctcggtttaaaaagcttctatgcctgaataggtgaccggaggtcggcacctttcctttgcaattactgaccacgccaccatggagaagggccctgtgcgggcaccggcggagaagccgcggggcgccaggtgcagcaatgggttccccgagcgggatccgccgcggcccgggcccagcaggccggcggagaagcccccgcggcccgaggccaagagcgcgcagcccgcggacggctggaagggcgagcggccccgcagcgaggaggataacgagctgaacctccctaacctggcagccgcctactcgtccatcctgagctcgctgggcgagaacccccagcggcaagggctgctcaagacgccctggagggcggcctcggccatgcagttcttcaccaagggctaccaggagaccatctcagatgtcctaaacgatgctatatttgatgaagatcatgatgagatggtgattgtgaaggacatagacatgttttccatgtgtgagcatcacttggttccatttgttggaaaggtccatattggttatcttcctaacaagcaagtccttggcctcagcaaacttgcgaggattgtagaaatctatagtagaagactacaagttcaggagcgccttacaaaacaaattgctgtagcaatcacggaagccttgcggcctgctggagtcggggtagtggttgaagcaacacacatgtgtatggtaatgcgaggtgtacagaaaatgaacagcaaaactgtgaccagcacaatgttgggtgtgttccgggaggatccaaagactcgggaagagttcctgactctcattaggagctgagccacctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccctggctgactgatacaatcgatttctggatccgcaggcctctgctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaagcttaacgcggtaaccacgtgcggacccaacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 15 - DNA, 6165 bp, Artificial Sequence
  Recombinant vector
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccggtcgcgtactagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcagatctttgtcgatcctaccatccactcgacacacccgccagcggccgctgccaagcttccgagctctcgaattcaaaggaggtacccaccatggccaccatgagccccgcggggcccaaggtcccctggttcccaagaaaagtgtcagagctggacaagtgtcatcacctggtcaccaagttcgaccctgacctggacttggaccacccgggcttctcggaccaggtgtaccgccagcgcaggaagctgattgctgagatcgccttccagtacaggcacggcgacccgattccccgtgtggagtacaccgccgaggagattgccacctggaaggaggtctacaccacgctgaagggcctctacgccacgcacgcctgcggggagcacctggaggcctttgctttgctggagcgcttcagcggctaccgggaagacaatatcccccagctggaggacgtctcccgcttcctgaaggagcgcacgggcttccagctgcggcctgtggccggcctgctgtccgcccgggacttcctggccagcctggccttccgcgtgttccagtgcacccagtatatccgccacgcgtcctcgcccatgcactcccctgagccggactgctgccacgagctgctggggcacgtgcccatgctggccgaccgcaccttcgcgcagttctcgcaggacattggcctggcgtccctgggggcctcggatgaggaaattgagaagctgtccacgctgtactggttcacggtggagttcgggctgtgtaagcagaacggggaggtgaaggcctatggtgccgggctgctgtcctcctacggggagctcctgcactgcctgtctgaggagcctgagattcgggccttcgaccctgaggctgcggccgtgcagccctaccaagaccagacgtaccagtcagtctacttcgtgtctgagagcttcagtgacgccaaggacaagctcaggagctatgcctcacgcatccagcgccccttctccgtgaagttcgacccgtacacgctggccatcgacgtgctggacagcccccaggccgtgcggcgctccctggagggtgtccaggatgagctggacacccttgcccatgcgctgagtgccattggctaacgcgcgaaacgcgcgccggtgaaacagaccctgaactttgatctgctgaaactggcgggcgatgtggaaagcaacccgggcccggccaccatggagaagggccctgtgcgggcaccggcggagaagccgcggggcgccaggtgcagcaatgggttccccgagcgggatccgccgcggcccgggcccagcaggccggcggagaagcccccgcggcccgaggccaagagcgcgcagcccgcggacggctggaagggcgagcggccccgcagcgaggaggataacgagctgaacctccctaacctggcagccgcctactcgtccatcctgagctcgctgggcgagaacccccagcggcaagggctgctcaagacgccctggagggcggcctcggccatgcagttcttcaccaagggctaccaggagaccatctcagatgtcctaaacgatgctatatttgatgaagatcatgatgagatggtgattgtgaaggacatagacatgttttccatgtgtgagcatcacttggttccatttgttggaaaggtccatattggttatcttcctaacaagcaagtccttggcctcagcaaacttgcgaggattgtagaaatctatagtagaagactacaagttcaggagcgccttacaaaacaaattgctgtagcaatcacggaagccttgcggcctgctggagtcggggtagtggttgaagcaacacacatgtgtatggtaatgcgaggtgtacagaaaatgaacagcaaaactgtgaccagcacaatgttgggtgtgttccgggaggatccaaagactcgggaagagttcctgactctcattaggagctgagccacctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccctggctgactgatacaatcgatttctggatccgcaggcctctgctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaagcttaacgcggtaaccacgtgcggacccaacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 16 - DNA, 6126 bp, Artificial Sequence
  Recombinant vector
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccggtcgcgtactagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcagatctttgtcgatcctaccatccactcgacacacccgccagcggccgctgccaagcttccgagctctcgaattcaaaggaggtacccaccatggccaccatgagccccgcggggcccaaggtcccctggttcccaagaaaagtgtcagagctggacaagtgtcatcacctggtcaccaagttcgaccctgacctggacttggaccacccgggcttctcggaccaggtgtaccgccagcgcaggaagctgattgctgagatcgccttccagtacaggcacggcgacccgattccccgtgtggagtacaccgccgaggagattgccacctggaaggaggtctacaccacgctgaagggcctctacgccacgcacgcctgcggggagcacctggaggcctttgctttgctggagcgcttcagcggctaccgggaagacaatatcccccagctggaggacgtctcccgcttcctgaaggagcgcacgggcttccagctgcggcctgtggccggcctgctgtccgcccgggacttcctggccagcctggccttccgcgtgttccagtgcacccagtatatccgccacgcgtcctcgcccatgcactcccctgagccggactgctgccacgagctgctggggcacgtgcccatgctggccgaccgcaccttcgcgcagttctcgcaggacattggcctggcgtccctgggggcctcggatgaggaaattgagaagctgtccacgctgtactggttcacggtggagttcgggctgtgtaagcagaacggggaggtgaaggcctatggtgccgggctgctgtcctcctacggggagctcctgcactgcctgtctgaggagcctgagattcgggccttcgaccctgaggctgcggccgtgcagccctaccaagaccagacgtaccagtcagtctacttcgtgtctgagagcttcagtgacgccaaggacaagctcaggagctatgcctcacgcatccagcgccccttctccgtgaagttcgacccgtacacgctggccatcgacgtgctggacagcccccaggccgtgcggcgctccctggagggtgtccaggatgagctggacacccttgcccatgcgctgagtgccattggctaaggaggtggcgggtccgggggcgggggtagcggtggcgggggctccgccaccatggagaagggccctgtgcgggcaccggcggagaagccgcggggcgccaggtgcagcaatgggttccccgagcgggatccgccgcggcccgggcccagcaggccggcggagaagcccccgcggcccgaggccaagagcgcgcagcccgcggacggctggaagggcgagcggccccgcagcgaggaggataacgagctgaacctccctaacctggcagccgcctactcgtccatcctgagctcgctgggcgagaacccccagcggcaagggctgctcaagacgccctggagggcggcctcggccatgcagttcttcaccaagggctaccaggagaccatctcagatgtcctaaacgatgctatatttgatgaagatcatgatgagatggtgattgtgaaggacatagacatgttttccatgtgtgagcatcacttggttccatttgttggaaaggtccatattggttatcttcctaacaagcaagtccttggcctcagcaaacttgcgaggattgtagaaatctatagtagaagactacaagttcaggagcgccttacaaaacaaattgctgtagcaatcacggaagccttgcggcctgctggagtcggggtagtggttgaagcaacacacatgtgtatggtaatgcgaggtgtacagaaaatgaacagcaaaactgtgaccagcacaatgttgggtgtgttccgggaggatccaaagactcgggaagagttcctgactctcattaggagctgagccacctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccctggctgactgatacaatcgatttctggatccgcaggcctctgctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaagcttaacgcggtaaccacgtgcggacccaacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 17 - DNA, 4561 bp, Artificial Sequence
  Recombinant vector
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccggtcgcgtctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgcaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttgcaccaaaatcaacgggactttccaaaatgtcgtaacaactccgccccattgacgcaaatgggcggtaggcgtgtacggtgggaggtctatataagcagagctgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaacatcgattgaattcagatccgctagtaatacgactcactatagggagaggatccggtaccgaggagatctgccgccgcgatcgccggcgcgccagatctcacgcttaactagctagcggaccgacgcgtacgcggccgctcgaggattataaggatgacgacgataaattcgtcgagcaccaccaccaccaccactaataaggtttatccgatccaccggatctagataagatatccgatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcggtaaccacgtgcggacccaacggccgcaggaacccctagtgatggagttggccactccctctagagctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 18 - DNA, 6773 bp, Artificial Sequence
  Genetic construct
ggcgatcgcggctcccgacatcttggaccattagctccacaggtatcttcttccctctagtggtcataacagcagcttcagctacctctcaattcaaaaaacccctcaagacccgtttagaggccccaaggggttatgctatcaatcgttgcgttacacacacaaaaaaccaacacacatccatcttcgatggatagcgattttattatctaactgctgatcgagtgtagccagatctagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcagatctagagatcccgggaccgccaccatgagccccgcggggcccaaggtcccctggttcccaagaaaagtgtcagagctggacaagtgtcatcacctggtcaccaagttcgaccctgacctggacttggaccacccgggcttctcggaccaggtgtaccgccagcgcaggaagctgattgctgagatcgccttccagtacaggcacggcgacccgattccccgtgtggagtacaccgccgaggagattgccacctggaaggaggtctacaccacgctgaagggcctctacgccacgcacgcctgcggggagcacctggaggcctttgctttgctggagcgcttcagcggctaccgggaagacaatatcccccagctggaggacgtctcccgcttcctgaaggagcgcacgggcttccagctgcggcctgtggccggcctgctgtccgcccgggacttcctggccagcctggccttccgcgtgttccagtgcacccagtatatccgccacgcgtcctcgcccatgcactcccctgagccggactgctgccacgagctgctggggcacgtgcccatgctggccgaccgcaccttcgcgcagttctcgcaggacattggcctggcgtccctgggggcctcggatgaggaaattgagaagctgtccacgctgtactggttcacggtggagttcgggctgtgtaagcagaacggggaggtgaaggcctatggtgccgggctgctgtcctcctacggggagctcctgcactgcctgtctgaggagcctgagattcgggccttcgaccctgaggctgcggccgtgcagccctaccaagaccagacgtaccagtcagtctacttcgtgtctgagagcttcagtgacgccaaggacaagctcaggagctatgcctcacgcatccagcgccccttctccgtgaagttcgacccgtacacgctggccatcgacgtgctggacagcccccaggccgtgcggcgctccctggagggtgtccaggatgagctggacacccttgcccatgcgctgagtgccattggctaaagcaggtttccccaactgacacaaaacgtgcaacttgaaactccgcctggtctttccaggtctagaggggtaacactttgtactgcgtttggctccacgctcgatccactggcgagtgttagtaacagcactgttgcttcgtagcggagcatgacggccgtgggaactcctccttggtaacaaggacccacggggccaaaagccacgcccacacgggcccgtcatgtgtgcaaccccagcacggcgactttactgcgaaacccactttaaagtgacattgaaactggtacccacacactggtgacaggctaaggatgcccttcaggtaccccgaggtaacacgcgacactcgggatctgagaaggggactggggcttctataaaagcgctcggtttaaaaagcttctatgcctgaataggtgaccggaggtcggcacctttcctttgcaattactgaccacgccaccatggagaagccgcggggagtcaggtgcaccaatgggttctccgagcgggagctgccgcggcccggggccagcccgcctgccgagaagtcccggccgcccgaggccaagggcgcacagccggccgacgcctggaaggcagggcggcaccgcagcgaggaggaaaaccaggtgaacctccccaaactggcggctgcttactcgtccattctgctctcgctgggcgaggacccccagcggcaggggctgctcaagacgccctggagggcggccaccgccatgcagtacttcaccaagggataccaggagaccatctcagatgtcctgaatgatgctatatttgatgaagatcatgacgagatggtgattgtgaaggacatagatatgttctccatgtgtgagcatcaccttgttccatttgtaggaagggtccatattggctatcttcctaacaagcaagtccttggtctcagtaaacttgccaggattgtagaaatctacagtagacgactacaagttcaagagcgcctcaccaaacagattgcggtggccatcacagaagccttgcagcctgctggcgttggagtagtgattgaagcgacacacatgtgcatggtaatgcgaggcgtgcagaaaatgaacagcaagactgtcactagcaccatgctgggcgtgttccgggaagaccccaagactcgggaggagttcctcacactaatcaggagctgaggccacctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttcccatatgcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcaggcgatctctcgatttcgatcaagacattcctttaatggtcttttctggacaccactaggggtcagaagtagttcatcaaactttcttccctccctaatctcattggttaccttgggctatcgaaacttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgagtattcaacatttccgtgtcgcccttattcccttttttgcggcattttgccttcctgtttttgctcacccagaaacgctggtgaaagtaaaagatgctgaagatcagttgggtgcgcgagtgggttacatcgaactggatctcaacagcggtaagatccttgagagttttcgccccgaagaacgctttccaatgatgagcacttttaaagttctgctatgtggcgcggtattatcccgtattgacgccgggcaagagcaactcggtcgccgcatacactattctcagaatgacttggttgagtattcaccagtcacagaaaagcatcttacggatggcatgacagtaagagaattatgcagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaaccttgcgtaaactattaactggcgaactacttactctagcttcccggcaacagttgatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaagatcccgaatcgtttaaactcgactctggctctatcgaatctccgtcgtttcgagcttacgcgaacagccgtggcgctcatttgctcgtcgggcatcgaatctcgtcagctatcgtcagcttacctttttggca

SEQ ID NO: 19 - DNA, 6390 bp, Artificial Sequence
  Genetic construct
ggcgatcgcggctcccgacatcttggaccattagctccacaggtatcttcttccctctagtggtcataacagcagcttcagctacctctcaattcaaaaaacccctcaagacccgtttagaggccccaaggggttatgctatcaatcgttgcgttacacacacaaaaaaccaacacacatccatcttcgatggatagcgattttattatctaactgctgatcgagtgtagccagatctagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcagatctagagatcccgggaccgccaccatgagccccgcggggcccaaggtcccctggttcccaagaaaagtgtcagagctggacaagtgtcatcacctggtcaccaagttcgaccctgacctggacttggaccacccgggcttctcggaccaggtgtaccgccagcgcaggaagctgattgctgagatcgccttccagtacaggcacggcgacccgattccccgtgtggagtacaccgccgaggagattgccacctggaaggaggtctacaccacgctgaagggcctctacgccacgcacgcctgcggggagcacctggaggcctttgctttgctggagcgcttcagcggctaccgggaagacaatatcccccagctggaggacgtctcccgcttcctgaaggagcgcacgggcttccagctgcggcctgtggccggcctgctgtccgcccgggacttcctggccagcctggccttccgcgtgttccagtgcacccagtatatccgccacgcgtcctcgcccatgcactcccctgagccggactgctgccacgagctgctggggcacgtgcccatgctggccgaccgcaccttcgcgcagttctcgcaggacattggcctggcgtccctgggggcctcggatgaggaaattgagaagctgtccacgctgtactggttcacggtggagttcgggctgtgtaagcagaacggggaggtgaaggcctatggtgccgggctgctgtcctcctacggggagctcctgcactgcctgtctgaggagcctgagattcgggccttcgaccctgaggctgcggccgtgcagccctaccaagaccagacgtaccagtcagtctacttcgtgtctgagagcttcagtgacgccaaggacaagctcaggagctatgcctcacgcatccagcgccccttctccgtgaagttcgacccgtacacgctggccatcgacgtgctggacagcccccaggccgtgcggcgctccctggagggtgtccaggatgagctggacacccttgcccatgcgctgagtgccattggctaacgcgcgaaacgcgcgccggtgaaacagaccctgaactttgatctgctgaaactggcgggcgatgtggaaagcaacccgggcccgatggagaagccgcggggagtcaggtgcaccaatgggttctccgagcgggagctgccgcggcccggggccagcccgcctgccgagaagtcccggccgcccgaggccaagggcgcacagccggccgacgcctggaaggcagggcggcaccgcagcgaggaggaaaaccaggtgaacctccccaaactggcggctgcttactcgtccattctgctctcgctgggcgaggacccccagcggcaggggctgctcaagacgccctggagggcggccaccgccatgcagtacttcaccaagggataccaggagaccatctcagatgtcctgaatgatgctatatttgatgaagatcatgacgagatggtgattgtgaaggacatagatatgttctccatgtgtgagcatcaccttgttccatttgtaggaagggtccatattggctatcttcctaacaagcaagtccttggtctcagtaaacttgccaggattgtagaaatctacagtagacgactacaagttcaagagcgcctcaccaaacagattgcggtggccatcacagaagccttgcagcctgctggcgttggagtagtgattgaagcgacacacatgtgcatggtaatgcgaggcgtgcagaaaatgaacagcaagactgtcactagcaccatgctgggcgtgttccgggaagaccccaagactcgggaggagttcctcacactaatcaggagctgaggccacctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttcccatatgcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcaggcgatctctcgatttcgatcaagacattcctttaatggtcttttctggacaccactaggggtcagaagtagttcatcaaactttcttccctccctaatctcattggttaccttgggctatcgaaacttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgagtattcaacatttccgtgtcgcccttattcccttttttgcggcattttgccttcctgtttttgctcacccagaaacgctggtgaaagtaaaagatgctgaagatcagttgggtgcgcgagtgggttacatcgaactggatctcaacagcggtaagatccttgagagttttcgccccgaagaacgctttccaatgatgagcacttttaaagttctgctatgtggcgcggtattatcccgtattgacgccgggcaagagcaactcggtcgccgcatacactattctcagaatgacttggttgagtattcaccagtcacagaaaagcatcttacggatggcatgacagtaagagaattatgcagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaaccttgcgtaaactattaactggcgaactacttactctagcttcccggcaacagttgatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaagatcccgaatcgtttaaactcgactctggctctatcgaatctccgtcgtttcgagcttacgcgaacagccgtggcgctcatttgctcgtcgggcatcgaatctcgtcagctatcgtcagcttacctttttggca

SEQ ID NO: 20 - DNA, 6380 bp, Artificial Sequence
  Genetic construct
ggcgatcgcggctcccgacatcttggaccattagctccacaggtatcttcttccctctagtggtcataacagcagcttcagctacctctcaattcaaaaaacccctcaagacccgtttagaggccccaaggggttatgctatcaatcgttgcgttacacacacaaaaaaccaacacacatccatcttcgatggatagcgattttattatctaactgctgatcgagtgtagccagatctagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcagatctagagatcccgggaccgccaccatgagccccgcggggcccaaggtcccctggttcccaagaaaagtgtcagagctggacaagtgtcatcacctggtcaccaagttcgaccctgacctggacttggaccacccgggcttctcggaccaggtgtaccgccagcgcaggaagctgattgctgagatcgccttccagtacaggcacggcgacccgattccccgtgtggagtacaccgccgaggagattgccacctggaaggaggtctacaccacgctgaagggcctctacgccacgcacgcctgcggggagcacctggaggcctttgctttgctggagcgcttcagcggctaccgggaagacaatatcccccagctggaggacgtctcccgcttcctgaaggagcgcacgggcttccagctgcggcctgtggccggcctgctgtccgcccgggacttcctggccagcctggccttccgcgtgttccagtgcacccagtatatccgccacgcgtcctcgcccatgcactcccctgagccggactgctgccacgagctgctggggcacgtgcccatgctggccgaccgcaccttcgcgcagttctcgcaggacattggcctggcgtccctgggggcctcggatgaggaaattgagaagctgtccacgctgtactggttcacggtggagttcgggctgtgtaagcagaacggggaggtgaaggcctatggtgccgggctgctgtcctcctacggggagctcctgcactgcctgtctgaggagcctgagattcgggccttcgaccctgaggctgcggccgtgcagccctaccaagaccagacgtaccagtcagtctacttcgtgtctgagagcttcagtgacgccaaggacaagctcaggagctatgcctcacgcatccagcgccccttctccgtgaagttcgacccgtacacgctggccatcgacgtgctggacagcccccaggccgtgcggcgctccctggagggtgtccaggatgagctggacacccttgcccatgcgctgagtgccattggcggaggtggcgggtccgggggcgggggtagcggtggcgggggctccgccaccatggagaagggccctgtgcgggcaccggcggagaagccgcggggcgccaggtgcagcaatgggttccccgagcgggatccgccgcggcccgggcccagcaggccggcggagaagcccccgcggcccgaggccaagagcgcgcagcccgcggacggctggaagggcgagcggccccgcagcgaggaggataacgagctgaacctccctaacctggcagccgcctactcgtccatcctgagctcgctgggcgagaacccccagcggcaagggctgctcaagacgccctggagggcggcctcggccatgcagttcttcaccaagggctaccaggagaccatctcagatgtcctaaacgatgctatatttgatgaagatcatgatgagatggtgattgtgaaggacatagacatgttttccatgtgtgagcatcacttggttccatttgttggaaaggtccatattggttatcttcctaacaagcaagtccttggcctcagcaaacttgcgaggattgtagaaatctatagtagaagactacaagttcaggagcgccttacaaaacaaattgctgtagcaatcacggaagccttgcggcctgctggagtcggggtagtggttgaagcaacacacatgtgtatggtaatgcgaggtgtacagaaaatgaacagcaaaactgtgaccagcacaatgttgggtgtgttccgggaggatccaaagactcgggaagagttcctgactctcattaggagctgagccacctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttcccatatgcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcaggcgatctctcgatttcgatcaagacattcctttaatggtcttttctggacaccactaggggtcagaagtagttcatcaaactttcttccctccctaatctcattggttaccttgggctatcgaaacttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgagtattcaacatttccgtgtcgcccttattcccttttttgcggcattttgccttcctgtttttgctcacccagaaacgctggtgaaagtaaaagatgctgaagatcagttgggtgcgcgagtgggttacatcgaactggatctcaacagcggtaagatccttgagagttttcgccccgaagaacgctttccaatgatgagcacttttaaagttctgctatgtggcgcggtattatcccgtattgacgccgggcaagagcaactcggtcgccgcatacactattctcagaatgacttggttgagtattcaccagtcacagaaaagcatcttacggatggcatgacagtaagagaattatgcagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaaccttgcgtaaactattaactggcgaactacttactctagcttcccggcaacagttgatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaagatcccgaatcgtttaaactcgactctggctctatcgaatctccgtcgtttcgagcttacgcgaacagccgtggcgctcatttgctcgtcgggcatcgaatctcgtcagctatcgtcagcttacctttttggca

SEQ ID NO: 21 - protein, 497 aa, Homo sapiens
MPTPDATTPQAKGFRRAVSELDAKQAEAIMSPRFIGRRQSLIEDARKEREAAVAAAAAAVPSEPGDPLEAVAFEEKEGKAVLNLLFSPRATKPSALSRAVKVFETFEAKIHHLETRPAQRPRAGGPHLEYFVRLEVRRGDLAALLSGVRQVSEDVRSPAGPKVPWFPRKVSELDKCHHLVTKFDPDLDLDHPGFSDQVYRQRRKLIAEIAFQYRHGDPIPRVEYTAEEIATWKEVYTTLKGLYATHACGEHLEAFALLERFSGYREDNIPQLEDVSRFLKERTGFQLRPVAGLLSARDFLASLAFRVFQCTQYIRHASSPMHSPEPDCCHELLGHVPMLADRTFAQFSQDIGLASLGASDEEIEKLSTLYWFTVEFGLCKQNGEVKAYGAGLLSSYGELLHCLSEEPEIRAFDPEAAAVQPYQDQTYQSVYFVSESFSDAKDKLRSYASRIQRPFSVKFDPYTLAIDVLDSPQAVRRSLEGVQDELDTLAHALSAIG

SEQ ID NO: 22 - protein, 342 aa, Homo sapiens
MSPAGPKVPWFPRKVSELDKCHHLVTKFDPDLDLDHPGFSDQVYRQRRKLIAEIAFQYRHGDPIPRVEYTAEEIATWKEVYTTLKGLYATHACGEHLEAFALLERFSGYREDNIPQLEDVSRFLKERTGFQLRPVAGLLSARDFLASLAFRVFQCTQYIRHASSPMHSPEPDCCHELLGHVPMLADRTFAQFSQDIGLASLGASDEEIEKLSTLYWFTVEFGLCKQNGEVKAYGAGLLSSYGELLHCLSEEPEIRAFDPEAAAVQPYQDQTYQSVYFVSESFSDAKDKLRSYASRIQRPFSVKFDPYTLAIDVLDSPQAVRRSLEGVQDELDTLAHALSAIG

SEQ ID NO: 23 - protein, 250 aa, Homo sapiens
MEKGPVRAPAEKPRGARCSNGFPERDPPRPGPSRPAEKPPRPEAKSAQPADGWKGERPRSEEDNELNLPNLAAAYSSILSSLGENPQRQGLLKTPWRAASAMQFFTKGYQETISDVLNDAIFDEDHDEMVIVKDIDMFSMCEHHLVPFVGKVHIGYLPNKQVLGLSKLARIVEIYSRRLQVQERLTKQIAVAITEALRPAGVGVVVEATHMCMVMRGVQKMNSKTVTSTMLGVFREDPKTREEFLTLIRS

SEQ ID NO: 24 - protein, 15 aa, Artificial Sequence
  Flexible linker sequence
GGGGSGGGGSGGGGS

SEQ ID NO: 25 - DNA, 663 bp, Artificial Sequence
  CMV promoter
acgcgtggagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgtcaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttgcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcc

SEQ ID NO: 26 - DNA, 1338 bp, Artificial Sequence
  OIPR sequence
attgggatcttcacacagcaggtaaggttgcgggccgggcctgggccgggtccgggccgggtattgcccgcctaatgagcgggcttttttttcttaccccttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggtaccccggcccgcactgacccctggtgttgctttttttttttaggccgcaagctgaagcgtgtcc

SEQ ID NO: 27 - DNA, 60 bp, Artificial Sequence
  E2A spacer sequence
cagtgtactaattatgctctcttgaaattggctggagatgttgagagcaaccctggacct

SEQ ID NO: 28 - DNA, 66 bp, Artificial Sequence
  F2A spacer sequence
gtgaaacagactttgaattttgaccttctcaagttggcgggagacgtggagtccaaccctggacct

SEQ ID NO: 29 - DNA, 57 bp, Artificial Sequence
  P2A spacer sequence
gccacgaacttctctctgttaaagcaagcaggagatgttgaagaaaaccccgggcct

SEQ ID NO: 30 - DNA, 54 bp, Artificial Sequence
  T2A spacer sequence
gagggcaggggaagtcttctaacatgcggggacgtggaggaaaatcccggcccc

SEQ ID NO: 31 - DNA, 9 bp, Artificial Sequence
  GSG spacer
ggaagcgga

SEQ ID NO: 32 - DNA, 435 bp, Homo sapiens
atgagcacggaaggtggtggccgtcgctgccaggcacaagtgtcccgccgcatctccttcagcgcgagccaccgattgtacagtaaatttctaagtgatgaagaaaacttgaaactgtttgggaaatgcaacaatccaaatggccatgggcacaattataaagttgtggtgacagtacatggagagattgaccctgctacgggaatggttatgaatctggctgatctcaaaaaatatatggaggaggcgattatgcagccccttgatcataagaatctggatatggatgtgccatactttgcagatgtggtgagcacgactgaaaatgtagctgtttatatctgggacaacctccagaaagttcttcctgtaggagttctttataaagtaaaagtatacgaaactgacaataatattgtggtttataaaggagaa

SEQ ID NO: 33 - protein, 145 aa, Homo sapiens
MSTEGGGRRCQAQVSRRISFSASHRLYSKFLSDEENLKLFGKCNNPNGHGHNYKVVVTVHGEIDPATGMVMNLADLKKYMEEAIMQPLDHKNLDMDVPYFADVVSTTENVAVYIWDNLQKVLPVGVLYKVKVYETDNNIVVYKGE

SEQ ID NO: 34 - DNA, 6244 bp, Artificial Sequence
  Recombinant vector
gcgatcgcggctcccgacatcttggaccattagctccacaggtatcttcttccctctagtggtcataacagcagcttcagctacctctcaattcaaaaaacccctcaagacccgtttagaggccccaaggggttatgctatcaatcgttgcgttacacacacaaaaaaccaacacacatccatcttcgatggatagcgattttattatctaactgctgatcgagtgtagccagatctagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcagatctttgtcgatcctaccatccactcgacacacccgccagcggccgctgccaagcttccgagctctcgaattcaaaggaggtacccaccatggccaccatgagccccgcggggcccaaggtcccctggttcccaagaaaagtgtcagagctggacaagtgtcatcacctggtcaccaagttcgaccctgacctggacttggaccacccgggcttctcggaccaggtgtaccgccagcgcaggaagctgattgctgagatcgccttccagtacaggcacggcgacccgattccccgtgtggagtacaccgccgaggagattgccacctggaaggaggtctacaccacgctgaagggcctctacgccacgcacgcctgcggggagcacctggaggcctttgctttgctggagcgcttcagcggctaccgggaagacaatatcccccagctggaggacgtctcccgcttcctgaaggagcgcacgggcttccagctgcggcctgtggccggcctgctgtccgcccgggacttcctggccagcctggccttccgcgtgttccagtgcacccagtatatccgccacgcgtcctcgcccatgcactcccctgagccggactgctgccacgagctgctggggcacgtgcccatgctggccgaccgcaccttcgcgcagttctcgcaggacattggcctggcgtccctgggggcctcggatgaggaaattgagaagctgtccacgctgtactggttcacggtggagttcgggctgtgtaagcagaacggggaggtgaaggcctatggtgccgggctgctgtcctcctacggggagctcctgcactgcctgtctgaggagcctgagattcgggccttcgaccctgaggctgcggccgtgcagccctaccaagaccagacgtaccagtcagtctacttcgtgtctgagagcttcagtgacgccaaggacaagctcaggagctatgcctcacgcatccagcgccccttctccgtgaagttcgacccgtacacgctggccatcgacgtgctggacagcccccaggccgtgcggcgctccctggagggtgtccaggatgagctggacacccttgcccatgcgctgagtgccattggccgcgcgaaacgcgcgccggtgaaacagaccctgaactttgatctgctgaaactggcgggcgatgtggaaagcaacccgggcccgatggagaagggccctgtgcgggcaccggcggagaagccgcggggcgccaggtgcagcaatgggttccccgagcgggatccgccgcggcccgggcccagcaggccggcggagaagcccccgcggcccgaggccaagagcgcgcagcccgcggacggctggaagggcgagcggccccgcagcgaggaggataacgagctgaacctccctaacctggcagccgcctactcgtccatcctgagctcgctgggcgagaacccccagcggcaagggctgctcaagacgccctggagggcggcctcggccatgcagttcttcaccaagggctaccaggagaccatctcagatgtcctaaacgatgctatatttgatgaagatcatgatgagatggtgattgtgaaggacatagacatgttttccatgtgtgagcatcacttggttccatttgttggaaaggtccatattggttatcttcctaacaagcaagtccttggcctcagcaaacttgcgaggattgtagaaatctatagtagaagactacaagttcaggagcgccttacaaaacaaattgctgtagcaatcacggaagccttgcggcctgctggagtcggggtagtggttgaagcaacacacatgtgtatggtaatgcgaggtgtacagaaaatgaacagcaaaactgtgaccagcacaatgttgggtgtgttccgggaggatccaaagactcgggaagagttcctgactctcattaggagctgagccacctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccctggctgactgatacaatcgatttctggatccgcaggcctctgctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagtcgtcagctatcctgcaggcgatctctcgatttcgatcaagacattcctttaatggtcttttctggacaccactaggggtcagaagtagttcatcaaactttcttccctccctaatctcattggttaccttgggctatcgaaacttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgattgaacaagatggattgcacgcaggttctccggcggcttgggtggagaggctattcggctatgactgggcacaacagacaatcggctgctctgatgccgccgtgttccggctgtcagcgcaggggcgtccggttctttttgtcaagaccgacctgtccggtgccctgaatgaactgcaagacgaggcagcgcggctatcgtggctggcgacgacgggcgttccttgcgcggctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgtctatgcccgacggcgaggatctcgtcgtgacccacggcgatgcctgcttgccgaatatcatggtggaaaatggccgcttttctggattcatcgactgtggccgtctgggtgtggcggaccgctatcaggacatagcgttggctacccgtgatattgctgaagagcttggcggcgaatgggctgaccgcttccttgtgctttacggtatcgccgcgcccgattcgcagcgcatcgccttctatcgccttcttgacgagttcttctgaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtttaaactcgactctggctctatcgaatctccgtcgtttcgagcttacgcgaacagccgtggcgctcatttgctcgtcgggcatcgaatctcgtcagctatcgtcagcttacctttttggca

SEQ ID NO: 35 - DNA, 3015 bp, Homo sapiens
ctgctaggggctgcttcccagctactcctcttggctccgtggcttgccttccagcctgtgtgctgtctggagagcctttaaagcctcacttccaccaactagaagtctctccccaaccctgccctgacctcaagtgcacctcttcaaagtcaggtttagcagctgcagctgggggccctgaatcccacccctgctgtcttccttgaagacagaagtgttgggagctgaggatctgggctagagactggctgtatgatccagagaagtagtgtgcttctgggcctcagatttcccttctgtagaacaggtttgtctgaaatggagaggttggtgctcctctgcagggcctagtgggagtcaccatgagtggttaaaagatccagcttgtcttttggtgagctttgagaggaggtaacagggctgagttctggaagcctgaccaagggcagacttaaggggcctcttggagttgttctcatcaaatggggatgggacacagctaaagtgcccagggcttctctgtgcccacagatgctttagatcttggcacagtgtggtctaccagctgtctctctctgtgtatatatatgtatttcatagacagtgtacagtggcctggtttgtgctatcaggctggatatggacagaggcaagagtttgtggcagcagttatctcccaagagagtccaaagacatcatgttttcaagtttaggccaggtgctacttgagagagctcagacacagacaaaggtctggagagcacatgtcctccacccccacctagcttctgttgcaagcacctccagccgagacaagagaacgaattaaaaagcaatatttgtgtcagtgtaagacatttgccgaaaggttaaatccacattcgtgttgctgcagagcagccccctatgcaggatttgttagatacagctccgtcctaccctgtgccagctgagcaaacgccaggctgggtggggtggaacccagcctgggtttgcctcaccctgcaatccccccagcaccctctaaaggaggaccctgtggtgggcatgcagacctagggactgggcatagataacctttgggtttgggcaacagcccccactcctcaggattgaaggctaaggtgcagccagctctgccttcatggtgggaatgtctccacgtgacccctttctgggctgtggagaacactcagagaagagtcctgggatgccaggcaggccagggatgtgctgggcatgttgagacaggagtgggctaagccagcagagttgctgacccaggaagagttcagaaaggggcatggaacatggggaggggtccatagtgagagagagcaggcagtgcagagtaaatagtccctgagctgggggttatgggatttgcaggagcttgctcagagaaggcagaggagagatgctgcgccaagctgggtatcacagagcctcagactcctggaacaggaactgtgggggtcaggtcagcaggggaggttagggagtgttccctttgtactgacttagcatttatcctgcttctaggggggaaggggggccagtgggggatgcacagcaaggcagtgatgtggcaggcagcctgcgggagctcctggttcctggtgtgaaaaagctgggaaggaagagggctgggtctggtaagtacagcaggcagttggctcctgagagtccaagccctgtctagagggtggagtgagatttcagagggagagctaaacggggtgggggctggggagtccaggcttctggctcctgctaatactcagtgtgctgggtcctcagaacctcagggtggccattttcagggtgagagctctgtcctttggcacttctgcagactccagtatccagaggaataaagatggtactcttcctcagttcccttagtgagaggacacctttctctgaagggcttgggcagttgtcctgaaccattgcctgaaggaaggacttgactccagggacatagaatgggctcagcataagtcccctgtagtagagaaaggtcccctctctggtctccttagagatcctgtttccttggctgaggaagctagggtggatctttgtgtaagtgggtgtggatgctcactggaaatcaaaaggccccttggtgttagaccttggggtgccatgggagagttgatcactgagtgcgcccttacatgggggccagctgagaatggggctgcctctagctcgagaccatgatgcagggagtgagtgggggagttcaggatactcttaactaaagcagaggtctgtccccccagggaggggaggtcagaagaccctagggagatgccaaaggctagggttggcaccatgttgcaggctgtgtcttcaaggagatgataatcagaggaatcgaacctgcaaaagtgggccagtcttagatacactatagaggaataatcttctgaaacattctgtgtctcataggacctgcctgaggacccagccccagtgccagcacatacactggggcagtgagtagatagtatactttgttacatgggctggggggacatggcctgtgccctggaggggacttgaagacatccaaaaagctagtgagagggctcctagatttatttgtctccaagggctatatatagccttcctaacatgaacccttgggtaatccagcatgggcgctcccatatgccctggtttgattagagagctctagatgtctcctgtcccagaacaccagccagcccctgtcttcatgtcgtgtctagggcggagggtgattcagaggcaggtgcctgcgacagtggatgcaattagatctaatgggacggaggcctctctcgtccgtcgccctcgctctgtgcccacccccgcctccctcaggcacagcaggcgtggagaggatgcgcaggaggtaggaggtgggggacccagaggggctttgacgtcagcctggcctttaagaggccgcctgcctggcaagggccgtggagacagaactcgggaccaccagcttgcact

SEQ ID NO: 36 - DNA, 1179 bp, Homo sapiens
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 37 - DNA, 232 bp, Homo sapiens
ggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagg

SEQ ID NO: 38 - DNA, 344 bp, Simian virus 40
ctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagct

SEQ ID NO: 39 - DNA, 505 bp, Homo sapiens
gggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagg

SEQ ID NO: 40 - DNA, 1178 bp, Homo sapiens
ggtgcagcggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaa

SEQ ID NO: 41 - DNA, 305 bp, Artificial Sequence
  CMV enhancer
gcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatg

SEQ ID NO: 42 - DNA, 237 bp, Simian virus 40
cgatggagcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagc

SEQ ID NO: 43 - DNA, 1733 bp, Artificial Sequence
  CMV promoter
ctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattg

SEQ ID NO: 44 - DNA, 798 bp, Artificial Sequence
  CMV promoter
ccaacctgaaaaaaagtgatttcaggcaggtgctccaggtaattaaacattaataccccaccaaccaaccatcccttaaacccttacctcttgctcagctaattacagcccggaggagaagggccgtcccgcccgctcacctgtgggagtaacgcggtcagtcagagccggggcgggcggcgcgaggcggcggcggagcggggcacggggcgaaggcagcgcgcagcgactcccgcccgccgcgcgcttcgctttttatagggccgccgccgccgccgcctcgccataaaaggaaactttcggagcgcgccgctctgattggctgccgccgcacctctccgcctcgccccgccccgcccctcgccccgccccgccccgcctggcgcgcgccccccccccccccccgcccccatcgctgcacaaaataattaaaaaataaataaatacaaaattgggggtggggaggggggggagatggggagagtgaagcagaacgtggggctcacctcgaccatggtaatagcgatgactaatacgtagatgtactgccaagtaggaaagtcccataaggtcatgtactgggcacaatgccaggcgggccatttaccgtcattgacgtcaatagggggcgtacttggcatatgatacacttgatgtactgccaagtgggcagtttaccgtaaatactccacccattgacgtcaatggaaagtccctattggcgttactattgacgtcaatgggcgggggtcgttgggcggtcagccaggcgggccatttaccgtaagttatgtaacg

(19) United States
(12) Patent Application Publication
(10) Pub. No.: US 2022/0280657 A1
(43) Pub. Date: Sep. 8, 2022

(54) Genetic Construct

(71) Applicants: Healing Genes LLC, New York, NY (US); Panacea Venture Healthcare Fund I, L.P., Shanghai (CN)

(72) Inventor: Michael McDonald, Guildford (GB)

(21) Appl. No.: 17/634,942

(22) PCT Filed: Aug. 11, 2020
(86) PCT No.: PCT/GB2020/051910

§ 371 Date: Feb. 11, 2022

(30) Foreign Application Priority Data
Aug. 12, 2019 (GB) 1911522.9

(51) Int. Cl. A61K 48/00; A61P 25/16

(52) U.S. Cl. CPC A61K 48/0075; A61K 48/005; A61P 25/16

(57) ABSTRACT

The invention relates to the use of genetic constructs, expression cassettes and recombinant vectors comprising such constructs and cassettes for gene therapy and methods for treating neurodegenerative disorders, such as Parkinson's disease (PD). The constructs comprise a promoter operably linked to a first coding sequence, which encodes tyrosine hydroxylase (TH), and a second coding sequence, which encodes GTP cyclohydrolase 1 (GCH1). The second coding sequence is 3′ to the first coding sequence, and the first and second coding sequences are part of a single operon, wherein the genetic construct does not encode aromatic amino acid decarboxylase (AADC). The construct is delivered to the cerebrospinal fluid (CSF) of the subject.

The present invention relates to the use of genetic constructs, expression cassettes and recombinant vectors comprising such constructs and cassettes for gene therapy and methods for treating neurodegenerative disorders, such as Parkinson's disease (PD).

Parkinson's disease is a neurodegenerative disease associated with the loss of dopamine-producing cells in the striatum. There are three enzymes which are necessary for the production of dopamine by brain cells: tyrosine hydroxylase (TH), GTP cyclohydrolase 1 (GCH1) and aromatic amino acid decarboxylase (AADC). TH and GCH1 regulate the production of L-DOPA (a precursor to dopamine) from tyrosine, and AADC converts L-DOPA to dopamine. The current treatment options for Parkinson's disease include oral administration of L-DOPA, which, in contrast to dopamine, is absorbed across the blood-brain barrier. This treatment is efficacious because AADC is still present in the brains of Parkinson's disease patients.

However, a problem with oral L-DOPA therapy is that it can lead to side effects, such as abnormal movement. These side effects are believed to be due to the fluctuation of levels of L-DOPA in the blood and brain caused by the short half-life of L-DOPA and the variable absorption across the gut mucosa and blood brain barrier resulting from competition with other amino acids for active transport (Lees, April 2008, The Importance of Steady-State plasma DOPA levels in reducing motor fluctuations in Parkinson's disease, Expert Roundtable Supplement, CNS Spectr 13:4 (Suppl 7) P4-7).

Many attempts have been made to formulate L-DOPA into a sustained release oral product that will deliver steady blood and brain levels of L-DOPA. These have not been successful. Currently, the most effective method for delivering steady plasma L-DOPA level requires constant slow infusion of a gel formulation of L-DOPA directly into the patient's jejunum via a tube through the patient's abdominal wall. The more stable plasma levels of L-DOPA result in significantly improved symptomatic control and reduced dyskinesias (Olanow et al Continuous intrajejunal infusion of levodopa-carbidopa intestinal gel for patients with advanced Parkinson's disease: a randomised, controlled, double-blind, double-dummy study. The Lancet Neurology Vol 13 Feb. 2014) However the lifelong requirement for a tube through the abdominal wall (with adverse events including dislodgement, kinking, blockage and infection), to carry a large pump and to refresh the supply of gel daily restrict use of this therapy and make it suboptimal especially for elderly patients with PD.

Many attempts, therefore, have, been made by multiple authors to restore dopamine levels in Parkinson's disease patients by targeting gene therapy directly into the most affected area of the brain, i.e. the striatum. Preclinical and clinical studies have shown some effect with various constructs (including mixtures of three AAV vectors delivering TH, GCH, and AADC (Muramatsu 10 Feb. 2002, Behavioral Recovery in a Primate Model of Parkinson's disease by Tripe Transduction of Striatal Cells with Adeno-Associated Viral (AAV) Vectors Expressing dopamine-Synthesizing Enzymes, Human Gene Therapy, 12: 345-354), a single tricistronic Lente vector with all three genes or a bicistronic AAV vector with just TH and GCH (WO2013/061076 and WO2010/055209). Rosenblad et al evaluated a bicistronic AAV expressing tyrosine hydroxylase and GCH1 administered directly to the striatum to produce L-DOPA However, there are numerous problems and complexities associated with injecting the gene therapy construct directly into the patient's striatum, including: (a) vector targeting, (b) achieving sufficient vector distribution throughout the striatum, (c) the need to treat the patient's brain bilaterally, (d) because the injection is into the brain tissue it needs to be done very slowly, by a process called convection enhanced delivery, to avoid damage, and also to avoid backflow along the outside of the needle, i.e. the path of least resistance, (e) multiple needle tracts are usually required and the process takes about 3-10 hours of neurosurgical time.

The present invention is seeking to address one or more problems inherent in the prior art.

The inventor has previously developed a novel genetic construct, based on AAV, which leads to improved production of GCH1 and TH, and hence is suitable for use with an improved method of treatment for neurodegenerative diseases, in particular diseases associated with catecholamine dysfunction, such as Parkinson's disease (WO 2018215787). The inventor has developed a novel method of using gene therapy to treat Parkinson's disease and other brain disorders involving reduced levels of dopamine. The invention uses gene therapy which does not require to be targeted to the striatum to increase substrate generally in the brain but achieve a selective targeted increase in of the desired neurotransmitter (dopamine) in the targeted area of the brain due to the innate selective regional distribution of AADC.

Based on this previous work, the inventor hypothesised that by injecting the AAV vector into the intrathecal space (i.e. into the cerebrospinal fluid), the challenges of delivering vector directly to the striatum would be avoided but that restoration of dopamine levels would still be targeted to relevant areas of brain with innate expression of AADC.

The inventor therefore performed a study in rats using two routes to administer constructs of the invention into the CSF, the first involving an injection into an intracerebral ventricle, and the second involving an injection into the cisterna magna.

To his surprise, the inventor observed that by delivering constructs of the invention into the cerebrospinal fluid (CSF), it is possible to produce surprisingly high levels of L-DOPA in the CSF and a subsequent decrease in intrastriatal dopamine in the striatum consistent with feedback inhibition via striatal dopamine receptors, thus demonstrating that increasing substrate in the brain using non-targeted gene therapy is able to achieve a more targeted effect due to the innate selective regional distribution of AADC.

Thus, according to a first aspect of the invention, there is provided a genetic construct comprising a promoter operably linked to a first coding sequence, which encodes tyrosine hydroxylase (TH), and a second coding sequence, which encodes GTP cyclohydrolase 1 (GCH1), wherein the second coding sequence is 3′ to the first coding sequence, and the first and second coding sequences are part of a single operon, and wherein the genetic construct does not encode aromatic amino acid decarboxylase (AADC), for use in treating, preventing, or ameliorating a neurodegenerative disorder in a subject, wherein the construct is delivered to the cerebrospinal fluid (CSF) of the subject.

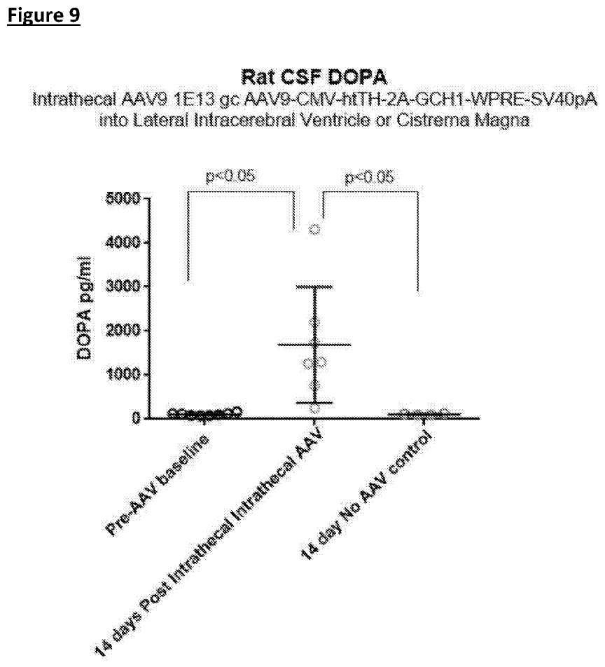

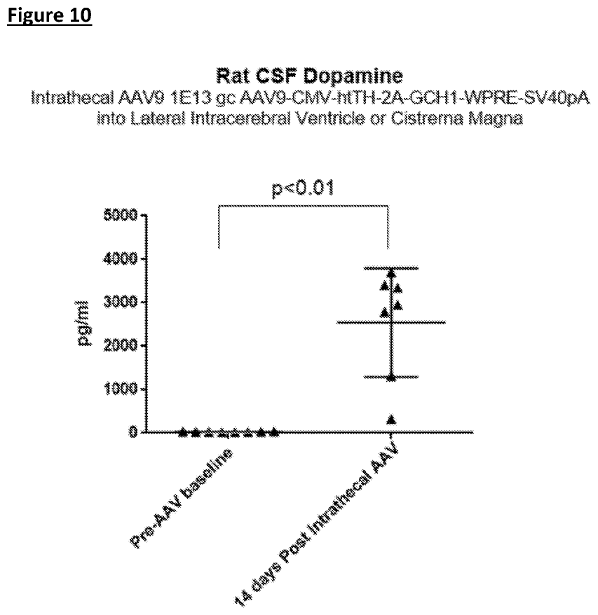

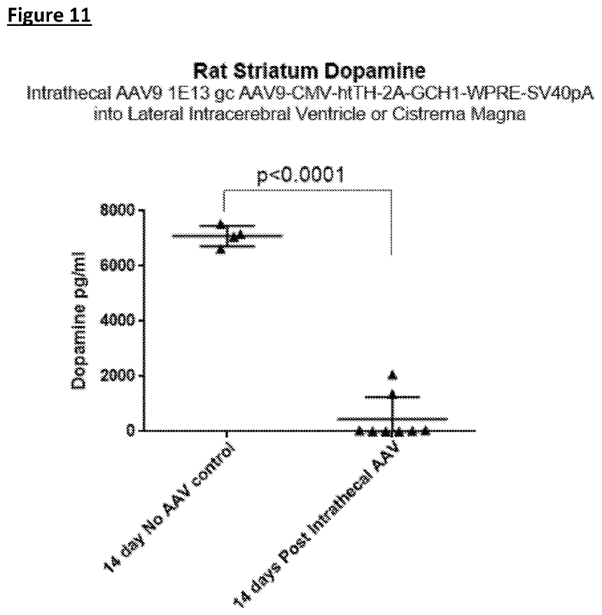

Advantageously, the inventors have identified a highly novel administration route for delivering the construct to a subject suffering from neurodegenerative disease, which results in a surprisingly effective approach for the treating the condition, such as Parkinson's disease (PD). As shown in FIG. 9, delivering the construct of the invention to the CSF results in surprisingly elevated L-DOPA concentrations in the CSF. Furthermore, as shown in FIG. 10, the L-DOPA in the CSF is being decarboxylated to dopamine in the CSF, by AADC. Moreover, FIG. 11 shows that intracellular dopamine levels in the striatum are also significantly reduced. This provides evidence that the L-DOPA and dopamine produced outside of the striatum, for example produced ectopically by the transduced ependyma and tissue adjacent to the CSF and to a lesser degree neurons throughout the brain, may be transported to the striatum by the blood supply and/or by pulsatile flow of extracellular fluid in the perivascular space. The increased level of dopamine and L-DOPA in the extracellular striatum restores local dopaminergic stimulation. The result demonstrates that the restoration is sufficient to produce feedback inhibition of additional local dopamine production within surviving dopaminergic cells. This implies that biologically effective levels have been reached.

Thus, by using the vector to increase the level of L-DOPA in the CSF and extracellular fluid of the brain, an alternative source L-DOPA substrate may be provided to enable partial of recomplete restoration of dopamine in regions in which dopa production is pathologically low but AADC activity is sufficient, such as the Parkinsonian striatum. CSF and brain levels would be more stable without the acute fluctuations experienced with oral L-DOPA therapy. Although the invention exposes the entire brain to an increased level of L-DOPA, over forty years of clinical experience with orally administered L-DOPA indicates that long term exposure of other areas of the brain to increased levels of DOPA is well tolerated. The peaks and troughs in brain L-DOPA levels inherent with oral therapy are avoided. This may result in reduced fluctuation of dopamine levels in the striatum and thus improved symptomatic control of Parkinson's disease (or other conditions due to reduced brain dopamine) and a reduced risk of L-DOPA induced dyskinesia.

Thus, L-DOPA produced by cells expressing the construct outside of the striatum, enters the CSF and diffuses from the CSF into striatal extracellular space, making it available for conversion by local residual AADC to dopamine to mitigate the symptoms PD.

Advantageously, delivering the gene therapy construct to the CSF ensures that the side effects associated with oral L-DOPA therapy, such as abnormal movement, can be avoided, because the variable absorption across the gut mucosa and blood brain barrier resulting from competition with other amino for active transport can be circumvented. In addition, it will be readily appreciated that delivery to the CSF is easier, safer and less time-consuming that injecting the gene therapy construct directly into the patient's striatum, as currently described in the prior art. Injection of the vector can be achieved in minutes rather than hours.

Preferably, the construct is delivered to the CSF by injection. One or more injections of the construct may be carried out to deliver the construct to the CSF. However, preferably the construct is delivered to the CSF by a single injection.

Preferably, the construct is delivered to the CSF by intrathecal injection. More preferably, the genetic construct is delivered to the CSF via one or more of a group selected from: the intracerebral ventricle system; the cisterna magna; and between lumbar vertebrae L3/L4, L4/L5 or L5/S1. More preferably, the genetic construct is delivered to the CSF via the intracerebral ventricle system or via the cisterna magna, preferably by a single injection.

In one embodiment, construct is delivered to the CSF via between lumbar vertebrae L3/L4, L4/L5 or L5/S1. Advantageously, adding a contrast agent to the injected composition enables effective delivery of the construct to the brain, wherein the increased mass associated with the contrast agent enables the construct to be transported to the brain when the head is lowered after injection of the genetic construct between lumbar vertebrae L3/L4, L4/L5 or L5/S1. A means of delivering the contrast agent, and therefore the construct of the invention, to the brain may be by use of a Trendelenburg tilting table, such method is well known to those skilled in the art. Thus, the use may comprise tilting the patent between about 10 and 40 degrees, preferably about 15 and 30 degrees head-down, i.e. supine with the feet being elevated above the head, during infusion of the contrast agent and construct.

Accordingly, the use may further comprise injecting a contrast media in combination with the genetic construct of the invention.

The contrast media may be any suitable non-ionic, water-soluble contrast media, which would be known to those skilled in the art. Preferably, the contrast media may be iohexol, which the skilled person would understand may be referred to as Omnipaque 180™.

The inventor was especially surprised to observe that it is not required to target striatal cells with the construct of the invention, and that delivering the construct to the CSF results in uptake of the construct by cells outside of the striatum, for example ependymal and/or leptomeningeal cells surrounding the CSF. It is also known that intracisternal AAV9 transduces neurons and astrocytes throughout most regions of the brain and spinal cord outside of the striatum. The transduced cells may then produce and release L-DOPA into the CSF, blood and extracellular fluid, which may be transported to the striatum. This results in a selective increase in dopamine production in the striatum with intrinsic expression of AADC.

Thus, in one embodiment, the construct is substantively expressed by cells outside of the striatum. Preferably the construct is expressed by cells outside of the striatum. Thus, in one embodiment, the construct is expressed by ependymal cells, leptomeningeal cells, and/or neurons and astrocytes throughout the brain and spinal cord. More preferably, the construct is expressed by ependymal cells and/or leptomeningeal cells. In another embodiment the construct is not selectively expressed by cells of the striatum. Preferably the construct is not substantively expressed by cells of the striatum. More preferably, the construct is not expressed by cells of the striatum.

Preferably, the CSF DOPA level is increased sufficiently to trigger feedback inhibition of dopamine production by surviving dopaminergic cells within the striatum. The skilled person would understand that feedback inhibition of dopamine by surviving dopaminergic cells within the striatum may indicate that physiological or pharmacologically relevant levels of dopamine have been achieved. In one embodiment, the CSF DOPA level may be increased to between 5 pmol/ml and 20 pmol/ml. Preferably, the CSF DOPA level may be increased to between 7 pmol/ml and 15 pmol/ml. Most preferably, the CSF DOPA level may be increased to between 8 pmol/ml and 12 pmol/ml. The skilled person would understand that “pmol” refers to 10⁻¹² mol/ml.

In one embodiment, the neurodegenerative disorder to be treated is a disease associated with catecholamine dysfunction. In a preferred embodiment, the catecholamine dysfunction may be characterised by a dopamine deficiency. In another embodiment, the disorder to be treated is selected from the group consisting of Parkinson's disease, DOPA responsive dystonia, vascular Parkinsonism, side effects associated with L-DOPA treatment, or L-DOPA induced dyskinesia.

In a more preferred embodiment, the neurodegenerative disorder to be treated is Parkinson's disease.

In one embodiment, the first coding sequence comprises a nucleotide sequence encoding human TH. The nucleotide sequence encoding human TH is referred to herein as SEQ ID No:1, or a fragment or variant thereof, as set out below:

[SEQ ID NO: 1]
atgcccacccccgacgccaccacgccacaggccaagggcttccgcagggccgtgtctgagctggacgccaagcaggca
gaggccatcatgtccccgcggttcattgggcgcaggcagagcctcatcgaggacgcccgcaaggagcgggaggcggcg
gtggcagcagcggccgctgcagtcccctcggagcccggggaccccctggaggctgtggcctttgaggagaaggagggg
aaggccgtgctaaacctgctcttctccccgagggccaccaagccctcggcgctgtcccgagctgtgaaggtgtttgag
acgtttgaagccaaaatccaccatctagagacccggcccgcccagaggccgcgagctgggggcccccacctggagtac
ttcgtgcgcctcgaggtgcgccgaggggacctggccgccctgctcagtggtgtgcgccaggtgtcagaggacgtgcgc
agccccgcggggcccaaggtcccctggttcccaagaaaagtgtcagagctggacaagtgtcatcacctggtcaccaag
ttcgaccctgacctggacttggaccacccgggcttctcggaccaggtgtaccgccagcgcaggaagctgattgctgag
atcgccttccagtacaggcacggcgacccgattccccgtgtggagtacaccgccgaggagattgccacctggaaggag
gtctacaccacgctgaagggcctctacgccacgcacgcctgcggggagcacctggaggcctttgctttgctggagcgc
ttcagcggctaccgggaagacaatatcccccagctggaggacgtctcccgcttcctgaaggagcgcacgggcttccag
ctgcggcctgtggccggcctgctgtccgcccgggacttcctggccagcctggccttccgcgtgttccagtgcacccag
tatatccgccacgcgtcctcgcccatgcactcccctgagccggactgctgccacgagctgctggggcacgtgcccatg
ctggccgaccgcaccttcgcgcagttctcgcaggacattggcctggcgtccctgggggcctcggatgaggaaattgag
aagctgtccacgctgtactggttcacggtggagttcgggctgtgtaagcagaacggggaggtgaaggcctatggtgcc
gggctgctgtcctcctacggggagctcctgcactgcctgtctgaggagcctgagattcgggccttcgaccctgaggct
gcggccgtgcagccctaccaagaccagacgtaccagtcagtctacttcgtgtctgagagcttcagtgacgccaaggac
aagctcaggagctatgcctcacgcatccagcgccccttctccgtgaagttcgacccgtacacgctggccatcgacgtg
ctggacagcccccaggccgtgcggcgctccctggagggtgtccaggatgagctggacacccttgcccatgcgctgagt
gccattggctag

Preferably, therefore, the first coding sequence comprises a nucleotide sequence substantially as set out in SEQ ID No:1, or a fragment or variant thereof.

In one preferred embodiment, the first coding sequence comprises a nucleotide sequence encoding human TH. Human TH may have an amino acid sequence according to NCBI Reference Sequence: NP_000351.2, which is referred to herein as SEQ ID NO: 21, or a fragment or variant thereof, as set out below:

[SEQ ID NO: 21]
MPTPDATTPQAKGFRRAVSELDAKQAEAIMSPRFIGRRQSLIEDARKEREAAVAAAAAAVPSEPGDPLEAVAFEEKEG
KAVLNLLFSPRATKPSALSRAVKVFETFEAKIHHLETRPAQRPRAGGPHLEYFVRLEVRRGDLAALLSGVRQVSEDVR
SPAGPKVPWFPRKVSELDKCHHLVTKFDPDLDLDHPGFSDQVYRQRRKLIAEIAFQYRHGDPIPRVEYTAEEIATWKE
VYTTLKGLYATHACGEHLEAFALLERFSGYREDNIPQLEDVSRFLKERTGFQLRPVAGLLSARDFLASLAFRVFQCTQ
YIRHASSPMHSPEPDCCHELLGHVPMLADRTFAQFSQDIGLASLGASDEEIEKLSTLYWFTVEFGLCKQNGEVKAYGA
GLLSSYGELLHCLSEEPEIRAFDPEAAAVQPYQDQTYQSVYFVSESFSDAKDKLRSYASRIQRPFSVKFDPYTLAIDV
LDSPQAVRRSLEGVQDELDTLAHALSAIG*

Preferably, therefore, the first coding sequence comprises a nucleotide sequence encoding an amino acid sequence substantially as set out in SEQ ID No:21, or a fragment or variant thereof.

In another embodiment, the first coding sequence comprises a nucleotide sequence encoding human truncated TH. Human truncated TH is a variant of TH with only the catalytic domain, and with the regulatory domain removed. The domains of TH and their roles are described in Daubner et al. (Daubner S C, Lohse D L, Fitzpatrick' P F. Expression and characterization of catalytic and regulatory domains of rat tyrosine hydroxylase.

hydroxylase. Protein Sci. 1993; 2:1452-60). Human truncated TH comprises the nucleotide sequence referred to herein as SEQ ID No:2, or a fragment or variant thereof, as set out below:

[SEQ ID NO: 2]
atgagccccgcggggcccaaggtcccctggttcccaagaaaagtgtcagagctggacaagtgtcatcacc
tggtcaccaagttcgaccctgacctggacttggaccacccgggcttctcggaccaggtgtaccgccagcg
caggaagctgattgctgagatcgccttccagtacaggcacggcgacccgattccccgtgtggagtacacc
gccgaggagattgccacctggaaggaggtctacaccacgctgaagggcctctacgccacgcacgcctgcg
gggagcacctggaggcctttgctttgctggagcgcttcagcggctaccgggaagacaatatcccccagct
ggaggacgtctcccgcttcctgaaggagcgcacgggcttccagctgcggcctgtggccggcctgctgtcc
gcccgggacttcctggccagcctggccttccgcgtgttccagtgcacccagtatatccgccacgcgtcct
cgcccatgcactcccctgagccggactgctgccacgagctgctggggcacgtgcccatgctggccgaccg
caccttcgcgcagttctcgcaggacattggcctggcgtccctgggggcctcggatgaggaaattgagaag
ctgtccacgctgtactggttcacggtggagttcgggctgtgtaagcagaacggggaggtgaaggcctatg
gtgccgggctgctgtcctcctacggggagctcctgcactgcctgtctgaggagcctgagattcgggcctt
cgaccctgaggctgcggccgtgcagccctaccaagaccagacgtaccagtcagtctacttcgtgtctgag
agcttcagtgacgccaaggacaagctcaggagctatgcctcacgcatccagcgccccttctccgtgaagt
tcgacccgtacacgctggccatcgacgtgctggacagcccccaggccgtgcggcgctccctggagggtgt
ccaggatgagctggacacccttgcccatgcgctgagtgccattggctag

Preferably, therefore, the first coding sequence comprises a nucleotide sequence substantially as set out in SEQ ID No:2, or a fragment or variant thereof.

In one preferred embodiment, the first coding sequence comprises a nucleotide sequence encoding human truncated TH. Human truncated TH comprises an amino acid sequence referred to herein as SEQ ID NO: 22, or a fragment or variant thereof, as set out below:

[SEQ ID NO: 22]
MSPAGPKVPWFPRKVSELDKCHHLVTKFDPDLDLDHPGFSDQVYRQRRKLIAEIAFQYRHGDPIPRVEYT
AEEIATWKEVYTTLKGLYATHACGEHLEAFALLERFSGYREDNIPQLEDVSRFLKERTGFQLRPVAGLLS
ARDFLASLAFRVFQCTQYIRHASSPMHSPEPDCCHELLGHVPMLADRTFAQFSQDIGLASLGASDEEIEK
LSTLYWFTVEFGLCKQNGEVKAYGAGLLSSYGELLHCLSEEPEIRAFDPEAAAVQPYQDQTYQSVYFVSE
SFSDAKDKLRSYASRIQRPFSVKFDPYTLAIDVLDSPQAVRRSLEGVQDELDTLAHALSAIG*

Preferably, therefore, the first coding sequence comprises a nucleotide sequence encoding an amino acid sequence substantially as set out in SEQ ID No:22, or a fragment or variant thereof.

In an embodiment, the second coding sequence comprises a nucleotide sequence encoding murine GCH1. The nucleotide sequence encoding murine GCH1 is referred to herein as SEQ ID No:3, or a fragment or variant thereof:

[SEQ ID NO: 3]
Ggtggttttcctttgaaaaacacgatgataatatggccacaaccgcggccgtagatcccgggaccatgga
gaagccgcggggagtcaggtgcaccaatgggttctccgagcgggagctgccgcggcccggggccagcccg
cctgccgagaagtcccggccgcccgaggccaagggcgcacagccggccgacgcctggaaggcagggcggc
accgcagcgaggaggaaaaccaggtgaacctccccaaactggcggctgcttactcgtccattctgctctc
gctgggcgaggacccccagcggcaggggctgctcaagacgccctggagggcggccaccgccatgcagtac
ttcaccaagggataccaggagaccatctcagatgtcctgaatgatgctatatttgatgaagatcatgacg
agatggtgattgtgaaggacatagatatgttctccatgtgtgagcatcaccttgttccatttgtaggaag
ggtccatattggctatcttcctaacaagcaagtccttggtctcagtaaacttgccaggattgtagaaatc
tacagtagacgactacaagttcaagagcgcctcaccaaacagattgcggtggccatcacagaagccttgc
agcctgctggcgttggagtagtgattgaagcgacacacatgtgcatggtaatgcgaggcgtgcagaaaat
gaacagcaagactgtcactagcaccatgctgggcgtgttccgggaagaccccaagactcgggaggagttc
ctcacactaatcaggagctgag

Therefore, the second coding sequence may comprise a nucleotide sequence substantially as set out in SEQ ID No:3, or a fragment or variant thereof.

In a preferred embodiment, the second coding sequence comprises a nucleotide sequence encoding human GCH1. For example, the sequence encoding human GCH may be the sequence according to GenBank NM 000161.2. The nucleotide sequence encoding human GCH1 is referred to herein as SEQ ID No:4, or a fragment or variant thereof, as set out below:

[SEQ ID NO: 4]
atggagaagggccctgtgcgggcaccggcggagaagccgcggggcgccagg
tgcagcaatgggttccccgagcgggatccgccgcggcccgggcccagcagg
ccggcggagaagcccccgcggcccgaggccaagagcgcgcagcccgcggac
ggctggaagggcgagcggccccgcagcgaggaggataacgagctgaacctc
cctaacctggcagccgcctactcgtccatcctgagctcgctgggcgagaac
ccccagcggcaagggctgctcaagacgccctggagggcggcctcggccatg
cagttcttcaccaagggctaccaggagaccatctcagatgtcctaaacgat
gctatatttgatgaagatcatgatgagatggtgattgtgaaggacatagac
atgttttccatgtgtgagcatcacttggttccatttgttggaaaggtccat
attggttatcttcctaacaagcaagtccttggcctcagcaaacttgcgagg
attgtagaaatctatagtagaagactacaagttcaggagcgccttacaaaa
caaattgctgtagcaatcacggaagccttgcggcctgctggagtcggggta
gtggttgaagcaacacacatgtgtatggtaatgcgaggtgtacagaaaatg
aacagcaaaactgtgaccagcacaatgttgggtgtgttccgggaggatcca
aagactcgggaagagttcctgactctcattaggagctga

Preferably, therefore, the second coding sequence comprises a nucleotide sequence substantially as set out in SEQ ID No: 4, or a fragment or variant thereof.

In one preferred embodiment, the second coding sequence comprises a nucleotide sequence encoding human GCH1. Human GCH1 may have an amino acid sequence according to NCBI Reference Sequence: NP_000152.1. Human GCH1 comprises an amino acid sequence referred to herein as SEQ ID NO: 23, or a fragment or variant thereof, as set out below:

[SEQ ID NO: 23]
MEKGPVRAPAEKPRGARCSNGFPERDPPRPGPSRPAEKPPRPEAKSAQPAD
GWKGERPRSEEDNELNLPNLAAAYSSILSSLGENPQRQGLLKTPWRAASAM
QFFTKGYQETISDVLNDAIFDEDHDEMVIVKDIDMFSMCEHHLVPFVGKVH
IGYLPNKQVLGLSKLARIVEIYSRRLQVQERLTKQIAVAITEALRPAGVGV
VVEATHMCMVMRGVQKMNSKTVTSTMLGVFREDPKTREEFLTLIRS*

Preferably, therefore, the second coding sequence comprises a nucleotide sequence encoding an amino acid sequence substantially as set out in SEQ ID No:23, or a fragment or variant thereof. 6-pyruvoyltetrahydropterin (PTPS) is the second rate-limiting enzyme after GCH1 that is necessary for the production of BH4, which is a cofactor essential for TH activity.

Thus, in one embodiment, the construct may further comprise a third coding sequence, which encodes 6-pyruvoyltetrahydropterin (PTPS), wherein the third coding sequence may be 3′ to the second coding sequence and is part of the a single operon.

In another embodiment, the PTPS sequence may be 5′ to the second coding sequence and is part of a single operon. For example, the third coding sequence may be 3′ of the first coding sequence and 5′ of the second coding sequence or the third coding sequence may be 5′ of the first coding sequence and 5′ the second coding sequence.

Preferably, the construct comprises a third coding sequence, which encodes 6-pyruvoyltetrahydropterin (PTPS), wherein the third coding sequence is 3′ to the second coding sequence and is part of the a single operon.

In one embodiment, the third coding sequence comprises a nucleotide sequence encoding human PTPS.

For example, the sequence encoding human PTPS may be the sequence according to GenBank NM000317. The nucleotide sequence encoding human PTPS is referred to herein as SEQ ID No: 32, or a fragment or variant thereof, as set out below:

[SEQ ID NO: 32]
atgagcacggaaggtggtggccgtcgctgccaggcacaagtgtcccgccgc
atctccttcagcgcgagccaccgattgtacagtaaatttctaagtgatgaa
gaaaacttgaaactgtttgggaaatgcaacaatccaaatggccatgggcac
aattataaagttgtggtgacagtacatggagagattgaccctgctacggga
atggttatgaatctggctgatctcaaaaaatatatggaggaggcgattatg
cagccccttgatcataagaatctggatatggatgtgccatactttgcagat
gtggtgagcacgactgaaaatgtagctgtttatatctgggacaacctccag
aaagttcttcctgtaggagttctttataaagtaaaagtatacgaaactgac
aataatattgtggtttataaaggagaa

Preferably, therefore, the third coding sequence comprises a nucleotide sequence substantially as set out in SEQ ID No: 32, or a fragment or variant thereof.

Human PTPS may have an amino acid sequence according to NCBI Reference Sequence: NP000308.1. Human PTPS comprises an amino acid sequence referred to herein as SEQ ID NO: 33, or a fragment or variant thereof, as set out below:

[SEQ ID NO: 33]
MSTEGGGRRCQAQVSRRISFSASHRLYSKFLSDEENLKLFGKCNNPNGHGH
NYKVVVTVHGEIDPATGMVMNLADLKKYMEEAIMQPLDHKNLDMDVPYFAD
VVSTTENVAVYIWDNLQKVLPVGVLYKVKVYETDNNIVVYKGE

Preferably, therefore, the third coding sequence a nucleotide sequence encoding an amino acid sequence substantially as set out in SEQ ID No:33, or a fragment or variant thereof.

The genetic construct according to the first aspect comprises a promoter. The promoter may be any suitable promoter, including a constitutive promoter, an activatable promoter, an inducible promoter, or a tissue-specific promoter. In a preferred embodiment, the promoter is a one enabling the generation of TH and GCH1 and optionally PTPS in the most suitable tissue or tissues for therapy. In an embodiment, the promoter is one that permits high expression in ependyma and neurons. The promoter may be a neuron-specific promoter.

In an embodiment, the promoter is the CMV promoter, one embodiment of which is referred to herein as SEQ ID NO: 25, as follows:

[SEQ ID No: 25]
ACGCGTGGAGCTAGTTATTAATAGTAATCAATTACGGGGTCATTAGTTCAT
AGCCCATATATGGAGTTCCGCGTTACATAACTTACGGTAAATGGCCCGCCT
GGCTGACCGCCCAACGACCCCCGCCCATTGACGTCAATAACGACGTATGTT
CCCATAGTAACGTCAATAGGGACTTTCCATTGACGTCAATGGGTGGAGTAT
TTACGGTAAACTGCCCACTTGGCAGTACATCAAGTGTATCATATGCCAAGT
ACGCCCCCTATTGACGTCAATGACGGTAAATGGCCCGCCTGGCATTATGCC
CAGTACATGACCTTATGGGACTTTCCTACTTGGCAGTACATCTACGTATTA
GTCATCGCTATTACCATGGCGATGCGGTTTTGGCAGTACATCAATGGGCGT
GGATAGCGGTTTGACTCACGGGGATTTCCAAGTCTCCACCCCATTGACGTC
AATGGGAGTTTGTTTTGCACCAAAATCAACGGGACTTTCCAAAATGTCGTA
ACAACTCCGCCCCATTGACGCAAATGGGCGGTAGGCGTGTACGGTGGGAGG
TCTATATAAGCAGAGCTCGTTTAGTGAACCGTCAGATCGCCTGGAGACGCC
ATCCACGCTGTTTTGACCTCCATAGAAGACACCGGGACCGATCCAGCCTCC

Preferably, therefore, the promoter may comprise a nucleotide sequence substantially as set out in SEQ ID No: 25, or a fragment or variant thereof.

In an embodiment, the promoter may be a human synapsin promoter. In an embodiment, the promoter is a human synapsin 1 promoter. One embodiment of the 469 nucleotide sequence encoding the human synapsin I (SYN I) promoter is referred to herein as SEQ ID NO: 5, as follows:

[SEQ ID NO: 5]
CTGCAGAGGGCCCTGCGTATGAGTGCAAGTGGGTTTTAGGACCAGGATGAG
GCGGGGTGGGGGTGCCTACCTGACGACCGACCCCGACCCACTGGACAAGCA
CCCAACCCCCATTCCCCAAATTGCGCATCCCCTATCAGAGAGGGGGAGGGG
AAACAGGATGCGGCGAGGCGCGTGCGCACTGCCAGCTTCAGCACCGCGGAC
AGTGCCTTCGCCCCCGCCTGGCGGCGCGCGCCACCGCCGCCTCAGCACTGA
AGGCGCGCTGACGTCACTCGCCGGTCCCCCGCAAACTCCCCTTCCCGGCCA
CCTTGGTCGCGTCCGCGCCGCCGCCGGCCCAGCCGGACCGCACCACGCGAG
GCGCGAGATAGGGGGGCACGGGCGCGACCATCTGCGCTGCGGCGCCGGCGA
CTCAGCGCTGCCTCAGTCTGCGGTGGGCAGCGGAGGAGTCGTGTCGTGCCT
GAGAGCGCAG

Preferably, therefore, the promoter may comprise a nucleotide sequence substantially as set out in SEQ ID No: 5, or a fragment or variant thereof.

In one embodiment, the promoter may be a tyrosine hydroxylase promoter, one embodiment of which is referred to herein as SEQ ID No: 35, as follows:

[SEQ ID No: 35]
CTGCTAGGGGCTGCTTCCCAGCTACTCCTCTTGGCTCCGTGGCTTGCCTTC
CAGCCTGTGTGCTGTCTGGAGAGCCTTTAAAGCCTCACTTCCACCAACTAG
AAGTCTCTCCCCAACCCTGCCCTGACCTCAAGTGCACCTCTTCAAAGTCAG
GTTTAGCAGCTGCAGCTGGGGGCCCTGAATCCCACCCCTGCTGTCTTCCTT
GAAGACAGAAGTGTTGGGAGCTGAGGATCTGGGCTAGAGACTGGCTGTATG
ATCCAGAGAAGTAGTGTGCTTCTGGGCCTCAGATTTCCCTTCTGTAGAACA
GGTTTGTCTGAAATGGAGAGGTTGGTGCTCCTCTGCAGGGCCTAGTGGGAG
TCACCATGAGTGGTTAAAAGATCCAGCTTGTCTTTTGGTGAGCTTTGAGAG
GAGGTAACAGGGCTGAGTTCTGGAAGCCTGACCAAGGGCAGACTTAAGGGG
CCTCTTGGAGTTGTTCTCATCAAATGGGGATGGGACACAGCTAAAGTGCCC
AGGGCTTCTCTGTGCCCACAGATGCTTTAGATCTTGGCACAGTGTGGTCTA
CCAGCTGTCTCTCTCTGTGTATATATATGTATTTCATAGACAGTGTACAGT
GGCCTGGTTTGTGCTATCAGGCTGGATATGGACAGAGGCAAGAGTTTGTGG
CAGCAGTTATCTCCCAAGAGAGTCCAAAGACATCATGTTTTCAAGTTTAGG
CCAGGTGCTACTTGAGAGAGCTCAGACACAGACAAAGGTCTGGAGAGCACA
TGTCCTCCACCCCCACCTAGCTTCTGTTGCAAGCACCTCCAGCCGAGACAA
GAGAACGAATTAAAAAGCAATATTTGTGTCAGTGTAAGACATTTGCCGAAA
GGTTAAATCCACATTCGTGTTGCTGCAGAGCAGCCCCCTATGCAGGATTTG
TTAGATACAGCTCCGTCCTACCCTGTGCCAGCTGAGCAAACGCCAGGCTGG
GTGGGGTGGAACCCAGCCTGGGTTTGCCTCACCCTGCAATCCCCCCAGCAC
CCTCTAAAGGAGGACCCTGTGGTGGGCATGCAGACCTAGGGACTGGGCATA
GATAACCTTTGGGTTTGGGCAACAGCCCCCACTCCTCAGGATTGAAGGCTA
AGGTGCAGCCAGCTCTGCCTTCATGGTGGGAATGTCTCCACGTGACCCCTT
TCTGGGCTGTGGAGAACACTCAGAGAAGAGTCCTGGGATGCCAGGCAGGCC
AGGGATGTGCTGGGCATGTTGAGACAGGAGTGGGCTAAGCCAGCAGAGTTG
CTGACCCAGGAAGAGTTCAGAAAGGGGCATGGAACATGGGGAGGGGTCCAT
AGTGAGAGAGAGCAGGCAGTGCAGAGTAAATAGTCCCTGAGCTGGGGGTTA
TGGGATTTGCAGGAGCTTGCTCAGAGAAGGCAGAGGAGAGATGCTGCGCCA
AGCTGGGTATCACAGAGCCTCAGACTCCTGGAACAGGAACTGTGGGGGTCA
GGTCAGCAGGGGAGGTTAGGGAGTGTTCCCTTTGTACTGACTTAGCATTTA
TCCTGCTTCTAGGGGGGAAGGGGGGCCAGTGGGGGATGCACAGCAAGGCAG
TGATGTGGCAGGCAGCCTGCGGGAGCTCCTGGTTCCTGGTGTGAAAAAGCT
GGGAAGGAAGAGGGCTGGGTCTGGTAAGTACAGCAGGCAGTTGGCTCCTGA
GAGTCCAAGCCCTGTCTAGAGGGTGGAGTGAGATTTCAGAGGGAGAGCTAA
ACGGGGTGGGGGCTGGGGAGTCCAGGCTTCTGGCTCCTGCTAATACTCAGT
GTGCTGGGTCCTCAGAACCTCAGGGTGGCCATTTTCAGGGTGAGAGCTCTG
TCCTTTGGCACTTCTGCAGACTCCAGTATCCAGAGGAATAAAGATGGTACT
CTTCCTCAGTTCCCTTAGTGAGAGGACACCTTTCTCTGAAGGGCTTGGGCA
GTTGTCCTGAACCATTGCCTGAAGGAAGGACTTGACTCCAGGGACATAGAA
TGGGCTCAGCATAAGTCCCCTGTAGTAGAGAAAGGTCCCCTCTCTGGTCTC
CTTAGAGATCCTGTTTCCTTGGCTGAGGAAGCTAGGGTGGATCTTTGTGTA
AGTGGGTGTGGATGCTCACTGGAAATCAAAAGGCCCCTTGGTGTTAGACCT
TGGGGTGCCATGGGAGAGTTGATCACTGAGTGCGCCCTTACATGGGGGCCA
GCTGAGAATGGGGCTGCCTCTAGCTCGAGACCATGATGCAGGGAGTGAGTG
GGGGAGTTCAGGATACTCTTAACTAAAGCAGAGGTCTGTCCCCCCAGGGAG
GGGAGGTCAGAAGACCCTAGGGAGATGCCAAAGGCTAGGGTTGGCACCATG
TTGCAGGCTGTGTCTTCAAGGAGATGATAATCAGAGGAATCGAACCTGCAA
AAGTGGGCCAGTCTTAGATACACTATAGAGGAATAATCTTCTGAAACATTC
TGTGTCTCATAGGACCTGCCTGAGGACCCAGCCCCAGTGCCAGCACATACA
CTGGGGCAGTGAGTAGATAGTATACTTTGTTACATGGGCTGGGGGGACATG
GCCTGTGCCCTGGAGGGGACTTGAAGACATCCAAAAAGCTAGTGAGAGGGC
TCCTAGATTTATTTGTCTCCAAGGGCTATATATAGCCTTCCTAACATGAAC
CCTTGGGTAATCCAGCATGGGCGCTCCCATATGCCCTGGTTTGATTAGAGA
GCTCTAGATGTCTCCTGTCCCAGAACACCAGCCAGCCCCTGTCTTCATGTC
GTGTCTAGGGCGGAGGGTGATTCAGAGGCAGGTGCCTGCGACAGTGGATGC
AATTAGATCTAATGGGACGGAGGCCTCTCTCGTCCGTCGCCCTCGCTCTGT
GCCCACCCCCGCCTCCCTCAGGCACAGCAGGCGTGGAGAGGATGCGCAGGA
GGTAGGAGGTGGGGGACCCAGAGGGGCTTTGACGTCAGCCTGGCCTTTAAG
AGGCCGCCTGCCTGGCAAGGGCCGTGGAGACAGAACTCGGGACCACCAGCT
TGCACT

Preferably, therefore, the promoter may comprise a nucleotide sequence substantially as set out in SEQ ID No: 35, or a fragment or variant thereof.

In one embodiment, the promoter may be a human eukaryotic translation elongation factor 1 alpha 1 promoter, one embodiment of which is referred to herein as SEQ ID No: 36, as follows:

[SEQ ID No: 36]
GGCTCCGGTGCCCGTCAGTGGGCAGAGCGCACATCGCCCACAGTCCCCGAG
AAGTTGGGGGGAGGGGTCGGCAATTGAACCGGTGCCTAGAGAAGGTGGCGC
GGGGTAAACTGGGAAAGTGATGTCGTGTACTGGCTCCGCCTTTTTCCCGAG
GGTGGGGGAGAACCGTATATAAGTGCAGTAGTCGCCGTGAACGTTCTTTTT
CGCAACGGGTTTGCCGCCAGAACACAGGTAAGTGCCGTGTGTGGTTCCCGC
GGGCCTGGCCTCTTTACGGGTTATGGCCCTTGCGTGCCTTGAATTACTTCC
ACCTGGCTGCAGTACGTGATTCTTGATCCCGAGCTTCGGGTTGGAAGTGGG
TGGGAGAGTTCGAGGCCTTGCGCTTAAGGAGCCCCTTCGCCTCGTGCTTGA
GTTGAGGCCTGGCCTGGGCGCTGGGGCCGCCGCGTGCGAATCTGGTGGCAC
CTTCGCGCCTGTCTCGCTGCTTTCGATAAGTCTCTAGCCATTTAAAATTTT
TGATGACCTGCTGCGACGCTTTTTTTCTGGCAAGATAGTCTTGTAAATGCG
GGCCAAGATCTGCACACTGGTATTTCGGTTTTTGGGGCCGCGGGCGGCGAC
GGGGCCCGTGCGTCCCAGCGCACATGTTCGGCGAGGCGGGGCCTGCGAGCG
CGGCCACCGAGAATCGGACGGGGGTAGTCTCAAGCTGGCCGGCCTGCTCTG
GTGCCTGGTCTCGCGCCGCCGTGTATCGCCCCGCCCTGGGCGGCAAGGCTG
GCCCGGTCGGCACCAGTTGCGTGAGCGGAAAGATGGCCGCTTCCCGGCCCT
GCTGCAGGGAGCTCAAAATGGAGGACGCGGCGCTCGGGAGAGCGGGCGGGT
GAGTCACCCACACAAAGGAAAAGGGCCTTTCCGTCCTCAGCCGTCGCTTCA
TGTGACTCCACGGAGTACCGGGCGCCGTCCAGGCACCTCGATTAGTTCTCG
AGCTTTTGGAGTACGTCGTCTTTAGGTTGGGGGGAGGGGTTTTATGCGATG
GAGTTTCCCCACACTGAGTGGGTGGAGACTGAAGTTAGGCCAGCTTGGCAC
TTGATGTAATTCTCCTTGGAATTTGCCCTTTTTGAGTTTGGATCTTGGTTC
ATTCTCAAGCCTCAGACAGTGGTTCAAAGTTTTTTTCTTCCATTTCAGGTG
TCGTGA

Preferably, therefore, the promoter may comprise a nucleotide sequence substantially as set out in SEQ ID No: 36, or a fragment or variant thereof.

In one embodiment, the promoter may be a human eukaryotic translation elongation factor 1 alpha 1 short form promoter, one embodiment of which is referred to herein as SEQ ID No: 37, as follows:

[SEQ ID No: 37]
GGCTCCGGTGCCCGTCAGTGGGCAGAGCGCACATCGCCCACAGTCCCCGAG
AAGTTGGGGGGAGGGGTCGGCAATTGATCCGGTGCCTAGAGAAGGTGGCGC
GGGGTAAACTGGGAAAGTGATGTCGTGTACTGGCTCCGCCTTTTTCCCGAG
GGTGGGGGAGAACCGTATATAAGTGCAGTAGTCGCCGTGAACGTTCTTTTT
CGCAACGGGTTTGCCGCCAGAACACAGG

Preferably, therefore, the promoter may comprise a nucleotide sequence substantially as set out in SEQ ID No: 37, or a fragment or variant thereof.

In one embodiment, the promoter may be a Simian virus 40 early promoter, one embodiment of which is referred to herein as SEQ ID No: 38, as follows:

[SEQ ID No: 38]
CTGTGGAATGTGTGTCAGTTAGGGTGTGGAAAGTCCCCAGGCTCCCCAGCA
GGCAGAAGTATGCAAAGCATGCATCTCAATTAGTCAGCAACCAGGTGTGGA
AAGTCCCCAGGCTCCCCAGCAGGCAGAAGTATGCAAAGCATGCATCTCAAT
TAGTCAGCAACCATAGTCCCGCCCCTAACTCCGCCCATCCCGCCCCTAACT
CCGCCCAGTTCCGCCCATTCTCCGCCCCATGGCTGACTAATTTTTTTTATT
TATGCAGAGGCCGAGGCCGCCTCTGCCTCTGAGCTATTCCAGAAGTAGTGA
GGAGGCTTTTTTGGAGGCCTAGGCTTTTGCAAAAAGCT

Preferably, therefore, the promoter may comprise a nucleotide sequence substantially as set out in SEQ ID No: 38, or a fragment or variant thereof.

In one embodiment, the promoter may be a human phosphoglycerated kinase 1 promoter, one embodiment of which is referred to herein as SEQ ID No: 39, as follows:

[SEQ ID No: 39]
GGGTTGCGCCTTTTCCAAGGCAGCCCTGGGTTTGCGCAGGGACGCGGCTGC
TCTGGGCGTGGTTCCGGGAAACGCAGCGGCGCCGACCCTGGGTCTCGCACA
TTCTTCACGTCCGTTCGCAGCGTCACCCGGATCTTCGCCGCTACCCTTGTG
GGCCCCCCGGCGACGCTTCCTGCTCCGCCCCTAAGTCGGGAAGGTTCCTTG
CGGTTCGCGGCGTGCCGGACGTGACAAACGGAAGCCGCACGTCTCACTAGT
ACCCTCGCAGACGGACAGCGCCAGGGAGCAATGGCAGCGCGCCGACCGCGA
TGGGCTGTGGCCAATAGCGGCTGCTCAGCAGGGCGCGCCGAGAGCAGCGGC
CGGGAAGGGGCGGTGCGGGAGGCGGGGTGTGGGGCGGTAGTGTGGGCCCTG
TTCCTGCCCGCGCGGTGTTCCGCATTCTGCAAGCCTCCGGAGCGCACGTCG
GCAGTCGGCTCCCTCGTTGACCGAATCACCGACCTCTCTCCCCAGG

Preferably, therefore, the promoter may comprise a nucleotide sequence substantially as set out in SEQ ID No: 39, or a fragment or variant thereof.

In one embodiment, the promoter may be a human ubiquitin C promoter, one embodiment of which is referred to herein as SEQ ID No: 40, as follows:

[SEQ ID No: 40]
GGTGCAGCGGCCTCCGCGCCGGGTTTTGGCGCCTCCCGCGGGCGCCCCCCT
CCTCACGGCGAGCGCTGCCACGTCAGACGAAGGGCGCAGCGAGCGTCCTGA
TCCTTCCGCCCGGACGCTCAGGACAGCGGCCCGCTGCTCATAAGACTCGGC
CTTAGAACCCCAGTATCAGCAGAAGGACATTTTAGGACGGGACTTGGGTGA
CTCTAGGGCACTGGTTTTCTTTCCAGAGAGCGGAACAGGCGAGGAAAAGTA
GTCCCTTCTCGGCGATTCTGCGGAGGGATCTCCGTGGGGCGGTGAACGCCG
ATGATTATATAAGGACGCGCCGGGTGTGGCACAGCTAGTTCCGTCGCAGCC
GGGATTTGGGTCGCGGTTCTTGTTTGTGGATCGCTGTGATCGTCACTTGGT
GAGTAGCGGGCTGCTGGGCTGGCCGGGGCTTTCGTGGCCGCCGGGCCGCTC
GGTGGGACGGAAGCGTGTGGAGAGACCGCCAAGGGCTGTAGTCTGGGTCCG
CGAGCAAGGTTGCCCTGAACTGGGGGTTGGGGGGAGCGCAGCAAAATGGCG
GCTGTTCCCGAGTCTTGAATGGAAGACGCTTGTGAGGCGGGCTGTGAGGTC
GTTGAAACAAGGTGGGGGGCATGGTGGGCGGCAAGAACCCAAGGTCTTGAG
GCCTTCGCTAATGCGGGAAAGCTCTTATTCGGGTGAGATGGGCTGGGGCAC
CATCTGGGGACCCTGACGTGAAGTTTGTCACTGACTGGAGAACTCGGTTTG
TCGTCTGTTGCGGGGGCGGCAGTTATGGCGGTGCCGTTGGGCAGTGCACCC
GTACCTTTGGGAGCGCGCGCCCTCGTCGTGTCGTGACGTCACCCGTTCTGT
TGGCTTATAATGCAGGGTGGGGCCACCTGCCGGTAGGTGTGCGGTAGGCTT
TTCTCCGTCGCAGGACGCAGGGTTCGGGCCTAGGGTAGGCTCTCCTGAATC
GACAGGCGCCGGACCTCTGGTGAGGGGAGGGATAAGTGAGGCGTCAGTTTC
TTTGGTCGGTTTTATGTACCTATCTTCTTAAGTAGCTGAAGCTCCGGTTTT
GAACTATGCGCTCGGGGTTGGCGAGTGTGTTTTGTGAAGTTTTTTAGGCAC
CTTTTGAAATGTAATCATTTGGGTCAATATGTAATTTTCAGTGTTAGACTA
GTAAA

Preferably, therefore, the promoter may comprise a nucleotide sequence substantially as set out in SEQ ID No: 40, or a fragment or variant thereof.

Preferably, therefore, the promoter may comprise a nucleotide sequence substantially as set out in SEQ ID No: 5, 25, 35 to 40, or a fragment or variant thereof.

Preferably, therefore, the promoter may comprise a nucleotide sequence substantially as set out in SEQ ID No: 5 or 25, or a fragment or variant thereof.

The genetic construct may further comprise one or more enhancer, which is configured to increase the expression of TH, GCH1 and optionally PTPS. In particular, the construct may comprise an enhancer designed to cooperate with the promoter. As an example, a construct including a CMV promoter may also include a CMV enhancer.

Thus, in one embodiment, the CMV promoter may comprise a CAG fused early enhancer, one embodiment of which is referred to herein as SEQ ID No: 43, as follows:

[SEQ ID No: 43]
ATGTTCCCATAGTAACGCCAATAGGGACTTTCCATTGACGTCAATGGGTGG
AGTATTTACGGTAAACTGCCCACTTGGCAGTACATCAAGTGTATCATATGC
CAAGTACGCCCCCTATTGACGTCAATGACGGTAAATGGCCCGCCTGGCATT
ATGCCCAGTACATGACCTTATGGGACTTTCCTACTTGGCAGTACATCTACG
TATTAGTCATCGCTATTACCATGGTCGAGGTGAGCCCCACGTTCTGCTTCA
CTCTCCCCATCTCCCCCCCCTCCCCACCCCCAATTTTGTATTTATTTATTT
TTTAATTATTTTGTGCAGCGATGGGGGCGGGGGGGGGGGGGGGGCGCGCGC
CAGGCGGGGCGGGGCGGGGCGAGGGGCGGGGCGGGGCGAGGCGGAGAGGTG
CGGCGGCAGCCAATCAGAGCGGCGCGCTCCGAAAGTTTCCTTTTATGGCGA
GGCGGCGGCGGCGGCGGCCCTATAAAAAGCGAAGCGCGCGGCGGGCGGGAG
TCGCTGCGCGCTGCCTTCGCCCCGTGCCCCGCTCCGCCGCCGCCTCGCGCC
GCCCGCCCCGGCTCTGACTGACCGCGTTACTCCCACAGGTGAGCGGGCGGG
ACGGCCCTTCTCCTCCGGGCTGTAATTAGCGCTTGGTTTAATGACGGCTTG
TTTCTTTTCTGTGGCTGCGTGAAAGCCTTGAGGGGCTCCGGGAGGGCCCTT
TGTGCGGGGGGAGCGGCTCGGGGGGTGCGTGCGTGTGTGTGTGCGTGGGGA
GCGCCGCGTGCGGCTCCGCGCTGCCCGGCGGCTGTGAGCGCTGCGGGCGCG
GCGCGGGGCTTTGTGCGCTCCGCAGTGTGCGCGAGGGGAGCGCGGCCGGGG
GCGGTGCCCCGCGGTGCGGGGGGGGCTGCGAGGGGAACAAAGGCTGCGTGC
GGGGTGTGTGCGTGGGGGGGTGAGCAGGGGGTGTGGGCGCGTCGGTCGGGC
TGCAACCCCCCCTGCACCCCCCTCCCCGAGTTGCTGAGCACGGCCCGGCTT
CGGGTGCGGGGCTCCGTACGGGGCGTGGCGCGGGGCTCGCCGTGCCGGGCG
GGGGGTGGCGGCAGGTGGGGGTGCCGGGCGGGGCGGGGCCGCCTCGGGCCG
GGGAGGGCTCGGGGGAGGGGCGCGGCGGCCCCCGGAGCGCCGGCGGCTGTC
GAGGCGCGGCGAGCCGCAGCCATTGCCTTTTATGGTAATCGTGCGAGAGGG
CGCAGGGACTTCCTTTGTCCCAAATCTGTGCGGAGCCGAAATCTGGGAGGC
GCCGCCGCACCCCCTCTAGCGGGCGCGGGGCGAAGCGGTGCGGCGCCGGCA
GGAAGGAAATGGGCGGGGAGGGCCTTCGTGCGTCGCCGCGCCGCCGTCCCC
TTCTCCCTCTCCAGCCTCGGGGCTGTCCGCGGGGGGACGGCTGCCTTCGGG
GGGGACGGGGCAGGGCGGGGTTCGGCTTCTGGCGTGTGACCGGCGGCTCTA
GAGCCTCTGCTAACCATGTTCATGCCTTCTTCTTTTTCCTACAGCTCCTGG
GCAACGTGCTGGTTATTGTGCTGTCTCATCATTTTGGCAAAGAATTG

Preferably, therefore, the promoter may comprise a nucleotide sequence substantially as set out in SEQ ID No: 43, or a variant or fragment thereof.

In one embodiment, the CMV promoter may comprise a CBh fused early enhancer, one embodiment of which is referred to herein as SEQ ID No: 44, as follows:

[SEQ ID No: 44]
CCAACCTGAAAAAAAGTGATTTCAGGCAGGTGCTCCAGGTAATTAAACATT
AATACCCCACCAACCAACCATCCCTTAAACCCTTACCTCTTGCTCAGCTAA
TTACAGCCCGGAGGAGAAGGGCCGTCCCGCCCGCTCACCTGTGGGAGTAAC
GCGGTCAGTCAGAGCCGGGGCGGGCGGCGCGAGGCGGCGGCGGAGCGGGGC
ACGGGGCGAAGGCAGCGCGCAGCGACTCCCGCCCGCCGCGCGCTTCGCTTT
TTATAGGGCCGCCGCCGCCGCCGCCTCGCCATAAAAGGAAACTTTCGGAGC
GCGCCGCTCTGATTGGCTGCCGCCGCACCTCTCCGCCTCGCCCCGCCCCGC
CCCTCGCCCCGCCCCGCCCCGCCTGGCGCGCGCCCCCCCCCCCCCCCCGCC
CCCATCGCTGCACAAAATAATTAAAAAATAAATAAATACAAAATTGGGGGT
GGGGAGGGGGGGGAGATGGGGAGAGTGAAGCAGAACGTGGGGCTCACCTCG
ACCATGGTAATAGCGATGACTAATACGTAGATGTACTGCCAAGTAGGAAAG
TCCCATAAGGTCATGTACTGGGCACAATGCCAGGCGGGCCATTTACCGTCA
TTGACGTCAATAGGGGGCGTACTTGGCATATGATACACTTGATGTACTGCC
AAGTGGGCAGTTTACCGTAAATACTCCACCCATTGACGTCAATGGAAAGTC
CCTATTGGCGTTACTATTGACGTCAATGGGCGGGGGTCGTTGGGCGGTCAG
CCAGGCGGGCCATTTACCGTAAGTTATGTAACG

Preferably, therefore, the promoter may comprise a nucleotide sequence substantially as set out in SEQ ID No: 44, or a variant or fragment thereof.

In one embodiment, the enhancer may be a CMV, one embodiment of which is referred to herein as SEQ ID No: 41, as follows:

[SEQ ID No: 41]
GCGTTACATAACTTACGGTAAATGGCCCGCCTGGCTGACCGCCCAACGACC
CCCGCCCATTGACGTCAATAATGACGTATGTTCCCATAGTAACGCCAATAG
GGACTTTCCATTGACGTCAATGGGTGGAGTATTTACGGTAAACTGCCCACT
TGGCAGTACATCAAGTGTATCATATGCCAAGTCCGCCCCCTATTGACGTCA
ATGACGGTAAATGGCCCGCCTGGCATTATGCCCAGTACATGACCTTACGGG
ACTTTCCTACTTGGCAGTACATCTACGTATTAGTCATCGCTATTACCATG

Preferably, therefore, the enhancer may comprise a nucleotide sequence substantially as set out in SEQ ID No: 41, or a variant or fragment thereof.

In one embodiment, the enhancer may be a Simian virus 40 enhancer, one embodiment of which is referred to herein as SEQ ID No: 42, as follows:

[SEQ ID No: 42]
CGATGGAGCGGAGAATGGGCGGAACTGGGCGGAGTTAGGGGCGGGATGGG
CGGAGTTAGGGGCGGGACTATGGTTGCTGACTAATTGAGATGCATGCTTT
GCATACTTCTGCCTGCTGGGGAGCCTGGGGACTTTCCACACCTGGTTGCT
GACTAATTGAGATGCATGCTTTGCATACTTCTGCCTGCTGGGGAGCCTGG
GGACTTTCCACACCCTAACTGACACACATTCCACAGC

Preferably, therefore, the enhancer may comprise a nucleotide sequence substantially as set out in SEQ ID No: 42, or a variant or fragment thereof.

In a preferred embodiment, the genetic construct comprises a spacer sequence disposed between the first and second coding sequences. This spacer sequence is such that it allows the production of functional TH and the production of functional GCH1 from the single promoter. In an embodiment, the spacer sequence comprises a sequence that allows for translation initiation in the middle of an mRNA sequence as part of the greater process of protein synthesis.

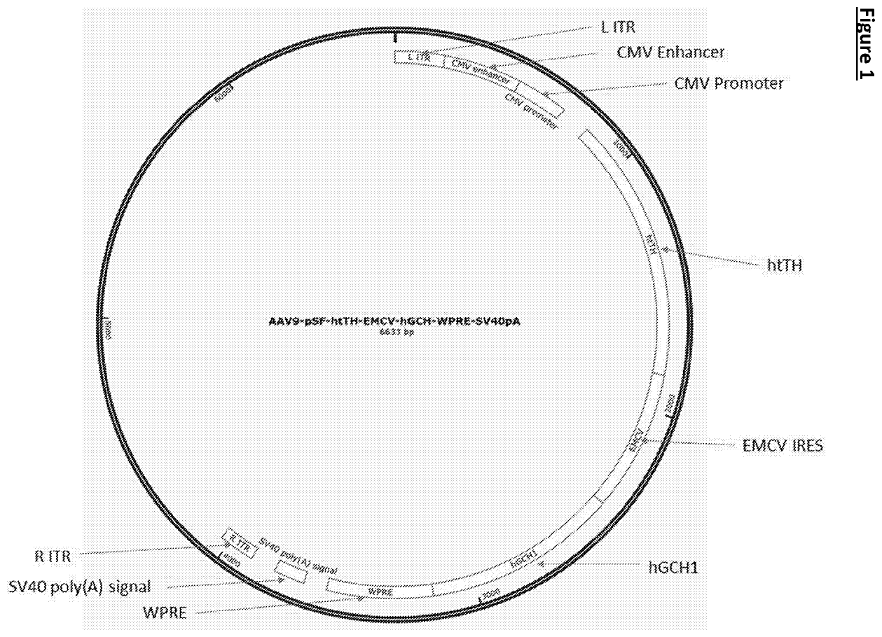

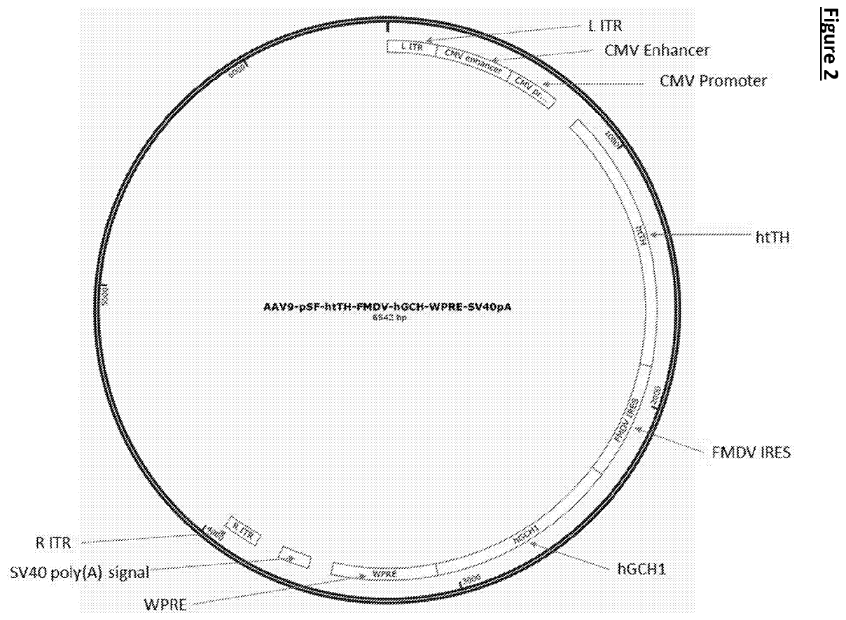

In a preferred embodiment, the spacer sequence may comprise a nucleotide sequence encoding a peptide spacer that is configured to be digested to thereby produce the TH and GCH1 as separate molecules. Preferably, in a particularly preferred embodiment, the spacer sequence comprises and encodes a viral peptide spacer sequence, more preferably a viral 2A peptide spacer sequence (Furler S, Paterna J-C, Weibel M and Bueler H Recombinant AAV vectors containing the foot and mouth disease virus 2A sequence confer efficient bicistronic gene expression in cultured cells and rat substantia nigra neurons Gene Ther. 2001, vol. 8, PP: 864-873). Preferably, the spacer sequence encoding the 2A peptide sequence connects the first coding sequence to the second coding sequence. This enables the construct to overcome the size restrictions that occur with expression in various vectors and enables expression of all of the peptides encoded by the construct of the first aspect to occur under control of a single promoter, as a single protein. Thus, following the translation of the single protein comprising the sequences of TH, the 2A peptide, and GCH1, cleavage occurs in the viral 2A peptide sequence at the terminal glycine-proline link, thereby liberating two proteins. The data presented herein demonstrate that a construct including a 5′ TH and a 3′ GCH1 separated by a viral 2A peptide spacer sequence leads to a surprisingly effective genetic construct (FIG. 1 and FIG. 2).

In a preferred embodiment, the spacer comprises a viral 2A peptide spacer and further comprises a furin cleavage site. Insertion of an upstream furin cleavage site allows the removal of 2A residues that would otherwise remain attached to the upstream protein.

In an embodiment, the nucleotide sequence of a peptide spacer encoding both a viral 2A sequence and a furin cleavage site may be referred to herein as SEQ ID No:8, or a fragment or variant thereof, as follows:

[SEQ ID NO: 8]
cgcgcgaaacgcgcgccggtgaaacagaccctgaactttgatctgctgaa
actggcgggcgatgtggaaagcaacccgggcccg

Preferably, therefore, the spacer sequence comprises a nucleotide sequence substantially as set out in SEQ ID No: 8, or a fragment or variant thereof.

The 2A spacer sequence may be any known variant, which includes those sequences referred to as E2A, F2A, P2A and T2A, as disclosed in Wang Y et al. Scientific Reports 2015, 5.

In one embodiment, the sequence is E2A, referred to herein as SEQ ID No: 27, as follows:

[SEQ ID NO: 27]
CAGTGTACTAATTATGCTCTCTTGAAATTGGCTGGAGATGTTGAGAGCAA
CCCTGGACCT

Preferably, therefore, the spacer sequence comprises a nucleotide sequence substantially as set out in SEQ ID No: 27, or a fragment or variant thereof.

In one embodiment, the sequence is F2A, referred to herein as SEQ ID No: 28, as follows:

[SEQ ID NO: 28]
GTGAAACAGACTTTGAATTTTGACCTTCTCAAGTTGGCGGGAGACGTGGA
GTCCAACCCTGGACCT

Preferably, therefore, the spacer sequence comprises a nucleotide sequence substantially as set out in SEQ ID No: 28, or a fragment or variant thereof.

In one embodiment, the sequence is P2A, referred to herein as SEQ ID No: 29, as follows:

[SEQ ID NO: 29]
GCCACGAACTTCTCTCTGTTAAAGCAAGCAGGAGATGTTGAAGAAAACCC
CGGGCCT

Preferably, therefore, the spacer sequence comprises a nucleotide sequence substantially as set out in SEQ ID No: 29, or a fragment or variant thereof.

In one embodiment, the sequence is T2A, referred to herein as SEQ ID No: 30, as follows:

[SEQ ID NO: 30]
GAGGGCAGGGGAAGTCTTCTAACATGCGGGGACGTGGAGGAAAATCCCGG
CCCC

Preferably, therefore, the spacer sequence comprises a nucleotide sequence substantially as set out in SEQ ID No: 30, or a fragment or variant thereof.

In an embodiment, the 2A sequence may be preceded by any sequence that improves the efficiency of 2A, i.e. the sequence is positioned 5′ to the 2A sequence. In an embodiment, the sequence that improves the efficiency of 2A is a glycine-serine-glycine spacer (GSG), referred to herein as SEQ ID No: 31, as follows:

[SEQ ID NO: 31]
GGAAGCGGA

Preferably, the 2A sequence is preceded by a nucleotide sequence substantially as set out in SEQ ID No: 31, or a fragment or variant thereof.

Alternatively, the spacer sequence may comprise a sequence encoding a flexible linker, which allows for the expression of both TH and GCH1 as a single polypeptide chain, but wherein the TH and GCH1 act as independent proteins. Hence, the proteins exert their effects in the same manner as if they were singly expressed. The data presented herein demonstrate that a construct including a 5′ TH and a 3′ GCH1 separated by spacer sequence comprising a flexible linker sequence leads to a surprisingly effective genetic construct (FIG. 1).

The flexible linker sequence may be as disclosed by WO 2013/061076 Ai (Oxford Biomedica), where this known linker was included in a tricistronic construct. The flexible linker sequence may be referred to herein as SEQ ID No:9, or a fragment or variant thereof, as follows:

[SEQ ID NO: 9]
ggaggtggcgggtccgggggcgggggtagcggtggcgggggctcc

Preferably, therefore, the flexible linker sequence comprises a nucleotide sequence substantially as set out in SEQ ID No: 9, or a fragment or variant thereof.

In one preferred embodiment, the flexible linker sequence comprises a nucleotide sequence encoding an amino acid sequence referred to herein as SEQ ID NO: 24, or a fragment or variant thereof, as set out below:

[SEQ ID NO: 24]
GGGGSGGGGSGGGGS

Preferably, therefore, the flexible linker sequence encodes an amino acid sequence substantially as set out in SEQ ID No: 24, or a fragment or variant thereof.

Alternatively, instead of a viral 2A spacer or flexible linker sequence, the spacer sequence may comprise an internal ribosome entry site (IRES). The data presented herein clearly demonstrate that a construct including a 5′ TH and a 3′ GCH1 separated by an IRES leads to a surprisingly effective genetic construct (FIG. 1 and FIG. 2). In an embodiment, the IRES is a picornavirus IRES.

In other embodiments, the IRES may be selected from a rhinovirus IRES, a hepatitis A virus IRES, a hepatitis C virus IRES, a poliovirus IRES, an enterovirus IRES, a cardiovirus IRES, an aphthovirus IRES, flavivirus IRES, a pestivirus IRES, a cripavirus IRES, a rhopalosiphum padi virus IRES, or any suitable IRES. In particular, the IRES may be any IRES described by the “IRESite” which provides a database of experimentally verified IRES structures (http://www.iresite.org/), or as disclosed in “New Messenger RNA Research Communications” (ISBN: 1-60021-488-6).

In a preferred embodiment, the IRES is a foot-and-mouth disease virus (FMDV) IRES, which may be as set out in SEQ ID No:6, or a fragment or variant thereof, as follows:

[SEQ ID NO: 6]
AGCAGGTTTCCCCAACTGACACAAAACGTGCAACTTGAAACTCCGCCTGG
TCTTTCCAGGTCTAGAGGGGTAACACTTTGTACTGCGTTTGGCTCCACGC
TCGATCCACTGGCGAGTGTTAGTAACACCACTGTTGCTTCGTAGCGGAGC
ATGACGGCCGTGGGAACTCCTCCTTGGTAACAAGGACCCACGGGGCCAAA
AGCCACGCCCACACGGGCCCGTCATGTGTGCAACCCCAGCACGGCGACTT
TACTGCGAAACCCACTTTAAAGTGACATTGAAACTGGTACCCACACACTG
GTGACAGGCTAAGGATGCCCTTCAGGTACCCCGAGGTAACACGCGACACT
CGGGATCTGAGAAGGGGACTGGGGCTTCTATAAAAGCGCTCGGTTTAAAA
AGCTTCTATGCCTGAATAGGTGACCGGAGGTCGGCACCTTTCCTTTGCAA
TTACTGACCAC

In another preferred embodiment, the IRES is an encephalomyocarditis virus (EMCV) IRES. The EMCV IRES may be as set out in SEQ ID No:7, or a fragment or variant thereof, as follows:

[SEQ ID NO: 7]
cgttactggccgaagccgcttggaataaggccggtgtgcgtttgtctata
tgttattttccaccatattgccgtcttttggcaatgtgagggcccggaaa
cctggccctgtcttcttgacgagcattcctaggggtctttcccctctcgc
caaaggaatgcaaggtctgttgaatgtcgtgaaggaagcagttcctctgg
aagcttcttgaagacaaacaacgtctgtagcgaccctttgcaggcagcgg
aaccccccacctggcgacaggtgcctctgcggccaaaagccacgtgtata
agatacacctgcaaaggcggcacaaccccagtgccacgttgtgagttgga
tagttgtggaaagagtcaaatggctcccctcaagcgtattcaacaagggg
ctgaaggatgcccagaaggtaccccattgtatgggatctgatctggggcc
tcggtgcacatgcttttcatgtgtttagtcgaggttaaaaaacgtctagg
ccccccgaaccacggggacgtggttttcctttgaaaaacacgatgataat
a

Therefore, preferably the IRES comprises a nucleotide sequence substantially as set out in SEQ ID No: 6 or 7, or a fragment or variant thereof.

In an embodiment where the third coding sequence is present, the genetic construct may further comprise a spacer sequence disposed between the second and third coding sequences. This spacer sequence allows the production of functional TH, the production of functional GCH1 and the production of functional PTPS from the single promoter. Preferably, the spacer sequence between the second and third coding sequence is as defined as the spacer sequence between the first and second coding sequence.

In an embodiment, the genetic construct may further comprise a nucleotide sequence encoding Woodchuck Hepatitis Virus Post-transcriptional Regulatory Element (WPRE), which enhances the expression of the two transgenes. Preferably, the WPRE coding sequence is disposed 3′ of the transgene coding sequence. In particular, the WPRE sequence is preferably 3′ of the GCH1 sequence. Preferably, when the third coding sequence is present, the WPRE sequence is preferably 3′ of the PTPS sequence.

One embodiment of the WPRE is 592 bp long, including gamma-alpha-beta elements, and is referred to herein as SEQ ID No: 10, as follows:

[SEQ ID NO: 10]
AATCAACCTCTGGATTACAAAATTTGTGAAAGATTGACTGGTATTCTTAA
CTATGTTGCTCCTTTTACGCTATGTGGATACGCTGCTTTAATGCCTTTGT
ATCATGCTATTGCTTCCCGTATGGCTTTCATTTTCTCCTCCTTGTATAAA
TCCTGGTTGCTGTCTCTTTATGAGGAGTTGTGGCCCGTTGTCAGGCAACG
TGGCGTGGTGTGCACTGTGTTTGCTGACGCAACCCCCACTGGTTGGGGCA
TTGCCACCACCTGTCAGCTCCTTTCCGGGACTTTCGCTTTCCCCCTCCCT
ATTGCCACGGCGGAACTCATCGCCGCCTGCCTTGCCCGCTGCTGGACAGG
GGCTCGGCTGTTGGGCACTGACAATTCCGTGGTGTTGTCGGGGAAGCTGA
CGTCCTTTCCATGGCTGCTCGCCTGTGTTGCCACCTGGATTCTGCGCGGG
ACGTCCTTCTGCTACGTCCCTTCGGCCCTCAATCCAGCGGACCTTCCTTC
CCGCGGCCTGCTGCCGGCTCTGCGGCCTCTTCCGCGTCTTCGCCTTCGCC
CTCAGACGAGTCGGATCTCCCTTTGGGCCGCCTCCCCGCCTG

Preferably, the WPRE comprises a nucleic acid sequence substantially as set out in SEQ ID No: 10, or a fragment or variant thereof.

However, in a preferred embodiment, a truncated WPRE is used, which is 247 bp long due to deletion of the beta element, and which is referred to herein as SEQ ID No: 11, as follows:

[SEQ ID NO: 11]
AATCAACCTCTGGATTACAAAATTTGTGAAAGATTGACTGGTATTCTTAA
CTATGTTGCTCCTTTTACGCTATGTGGATACGCTGCTTTAATGCCTTTGT
ATCATGCTATTGCTTCCCGTATGGCTTTCATTTTCTCCTCCTTGTATAAA
TCCTGGTTAGTTCTTGCCACGGCGGAACTCATCGCCGCCTGCCTTGCCCG
CTGCTGGACAGGGGCTCGGCTGTTGGGCACTGACAATTCCGTGGTGT

Preferably, the WPRE comprises a nucleic acid sequence substantially as set out in SEQ ID No: 11, or a fragment or variant thereof.

Preferably, the genetic construct comprises a nucleotide sequence encoding a polyA tail. Preferably, the polyA tail coding sequence is disposed 3′ of the transgene coding sequence, and preferably 3′ of the WHPE coding sequence.

Preferably, the polyA tail comprises the simian virus 40 poly-A 224 bp sequence. One embodiment of the polyA tail is referred to herein as SEQ ID No: 12, as follows:

[SEQ ID NO: 12]
AGCAGACATGATAAGATACATTGATGAGTTTGGACAAACCACAACTAGAA
TGCAGTGAAAAAAATGCTTTATTTGTGAAATTTGTGATGCTATTGCTTTA
TTTGTAACCATTATAAGCTGCAATAAACAAGTTAACAACAACAATTGCAT
TCATTTTATGTTTCAGGTTCAGGGGGAGGTGTGGGAGGTTTTTTAAAGCA
AGTAAAACCTCTACAAATGTGGTA

Preferably, the polyA tail comprises a nucleic acid sequence substantially as set out in PGP-SEQ ID No: 12, or a fragment or variant thereof.

Preferably, the genetic construct comprises left and/or right Inverted Terminal Repeat sequences (ITRs). Preferably, each ITR is disposed at the 5′ and/or 3′ end of the construct.

In a preferred embodiment the genetic construct may comprise, in this specified order, 5′ ITR; a human synapsin 1 promoter or a CMV promoter; a sequence encoding human truncated TH; a 2A-Furin-sequence; a sequence encoding human GCH1; a sequence encoding WPRE; a sequence encoding a poly A tail; and a 3′ ITR. The use of 5′ and 3′ indicates that the features are either upstream or downstream, and is not intended to indicate that the features are necessarily terminal features.

In a particular embodiment the genetic construct may comprise, in this specified order, a 5′ ITR; a human synapsin 1 promoter or a CMV promoter; a sequence encoding human truncated TH; a flexible linker; a sequence encoding human GCH1; a sequence encoding WPRE; a sequence encoding a poly A tail; and a 3′ ITR.

In a particular embodiment, the genetic construct comprises, in this specified order, a 5′ human synapsin 1 promoter or a CMV promoter; a sequence encoding human truncated TH; an IRES; and a 3′ sequence encoding human GCH1.

In a particular embodiment, the genetic construct may comprise, in this specified order, a 5′ ITR; a human synapsin 1 promoter or a CMV promoter; a sequence encoding human truncated TH; an IRES; a sequence encoding human GCH1; a sequence encoding WPRE; a sequence encoding a poly A tail; and a 3′ ITR.

The skilled person would understand that in embodiments whereby the sequence encoding human PTPS is present, the genetic construct may comprise sequences encoding human TH, GCH1 and PTPS in any 5′ to 3′ order, with any combination of linker sequence present between these sequences.

In a preferred embodiment the genetic construct may comprise, in this specified order, 5′ ITR; a human synapsin 1 promoter or a CMV promoter; a sequence encoding human truncated TH; a Furin-2A sequence; a sequence encoding human GCH1; a Furin-2A sequence; a sequence encoding human PTPS; a sequence encoding WPRE; a sequence encoding a poly A tail; and a 3′ ITR.

In a particular embodiment the genetic construct may comprise, in this specified order, a 5′ ITR; a human synapsin 1 promoter or a CMV promoter; a sequence encoding human truncated TH; a flexible linker; a sequence encoding human GCH1; a flexible linker; a sequence encoding human PTPS; a sequence encoding WPRE; a sequence encoding a poly A tail; and a 3′ ITR.

In a particular embodiment, the genetic construct comprises, in this specified order, a 5′ human synapsin 1 promoter or a CMV promoter; a sequence encoding human truncated TH; an IRES; a sequence encoding human GCH1, an IRES and a 3′ sequence encoding human PTPS. The use of 5′ and 3′ indicates that the features are either upstream or downstream, and is not intended to indicate that the features are necessarily terminal features.

In a particular embodiment, the genetic construct may comprise, in this specified order, a 5′ ITR; a human synapsin 1 promoter or a CMV promoter; a sequence encoding human truncated TH; an IRES; a sequence encoding human GCH1; an IRES; a sequence encoding human PTPS; a sequence encoding WPRE; a sequence encoding a poly A tail; and a 3′ ITR.

In a particular embodiment the genetic construct may comprise, in this specified order, 5′ ITR; a human synapsin 1 promoter or a CMV promoter; a sequence encoding human truncated TH; a Furin-2A sequence; a sequence encoding human PTPS; a Furin-2A sequence; a sequence encoding human GCH1; a sequence encoding WPRE; a sequence encoding a poly A tail; and a 3′ ITR.

In a particular embodiment the genetic construct may comprise, in this specified order, 5′ ITR; a human synapsin 1 promoter or a CMV promoter; a sequence encoding human PTPS; a Furin-2A sequence; a sequence encoding human truncated TH; a Furin-2A sequence; a sequence encoding human GCH1; a sequence encoding WPRE; a sequence encoding a poly A tail; and a 3′ ITR.

In a particular embodiment the genetic construct may comprise, in this specified order, 5′ ITR; a human synapsin 1 promoter or a CMV promoter; a sequence encoding human GCH1; a Furin-2A sequence; a sequence encoding human truncated TH; a Furin-2A sequence; a sequence encoding human PTPS; a sequence encoding WPRE; a sequence encoding a poly A tail; and a 3′ ITR.

One embodiment of the genetic construct is shown in FIG. 10, and is referred to herein as SEQ ID No: 18. This particular embodiment includes a CMV promoter and murine GCH1; these features would be easily replaceable by the skilled person for other variants as disclosed herein.

[SEQ ID NO: 18]
ggcgatcgcggctcccgacatcttggaccattagctccacaggtatcttcttccctctagtggtcataac
agcagcttcagctacctctcaattcaaaaaacccctcaagacccgtttagaggccccaaggggttatgct
atcaatcgttgcgttacacacacaaaaaaccaacacacatccatcttcgatggatagcgattttattatc
taactgctgatcgagtgtagccagatctagtaatcaattacggggtcattagttcatagcccatatatgg
agttccgcgttacataacttacggtaaatggcccgcctggctgaccgcccaacgacccccgcccattgac
gtcaataatgacgtatgttcccatagtaacgccaatagggactttccattgacgtcaatgggtggagtat
ttacggtaaactgcccacttggcagtacatcaagtgtatcatatgccaagtacgccccctattgacgtca
atgacggtaaatggcccgcctggcattatgcccagtacatgaccttatgggactttcctacttggcagta
catctacgtattagtcatcgctattaccatgctgatgcggttttggcagtacatcaatgggcgtggatag
cggtttgactcacggggatttccaagtctccaccccattgacgtcaatgggagtttgttttggcaccaaa
atcaacgggactttccaaaatgtcgtaacaactccgccccattgacgcaaatgggcggtaggcgtgtacg
gtgggaggtctatataagcagagctggtttagtgaaccgtcagatcagatctagagatcccgggaccgcc
accatgagccccgcggggcccaaggtcccctggttcccaagaaaagtgtcagagctggacaagtgtcatc
acctggtcaccaagttcgaccctgacctggacttggaccacccgggcttctcggaccaggtgtaccgcca
gcgcaggaagctgattgctgagatcgccttccagtacaggcacggcgacccgattccccgtgtggagtac
accgccgaggagattgccacctggaaggaggtctacaccacgctgaagggcctctacgccacgcacgcct
gcggggagcacctggaggcctttgctttgctggagcgcttcagcggctaccgggaagacaatatccccca
gctggaggacgtctcccgcttcctgaaggagcgcacgggcttccagctgcggcctgtggccggcctgctg
tccgcccgggacttcctggccagcctggccttccgcgtgttccagtgcacccagtatatccgccacgcgt
cctcgcccatgcactcccctgagccggactgctgccacgagctgctggggcacgtgcccatgctggccga
ccgcaccttcgcgcagttctcgcaggacattggcctggcgtccctgggggcctcggatgaggaaattgag
aagctgtccacgctgtactggttcacggtggagttcgggctgtgtaagcagaacggggaggtgaaggcct
atggtgccgggctgctgtcctcctacggggagctcctgcactgcctgtctgaggagcctgagattcgggc
cttcgaccctgaggctgcggccgtgcagccctaccaagaccagacgtaccagtcagtctacttcgtgtct
gagagcttcagtgacgccaaggacaagctcaggagctatgcctcacgcatccagcgccccttctccgtga
agttcgacccgtacacgctggccatcgacgtgctggacagcccccaggccgtgcggcgctccctggaggg
tgtccaggatgagctggacacccttgcccatgcgctgagtgccattggctaaagcaggtttccccaactg
acacaaaacgtgcaacttgaaactccgcctggtctttccaggtctagaggggtaacactttgtactgcgt
ttggctccacgctcgatccactggcgagtgttagtaacagcactgttgcttcgtagcggagcatgacggc
cgtgggaactcctccttggtaacaaggacccacggggccaaaagccacgcccacacgggcccgtcatgtg
tgcaaccccagcacggcgactttactgcgaaacccactttaaagtgacattgaaactggtacccacacac
tggtgacaggctaaggatgcccttcaggtaccccgaggtaacacgcgacactcgggatctgagaagggga
ctggggcttctataaaagcgctcggtttaaaaagcttctatgcctgaataggtgaccggaggtcggcacc
tttcctttgcaattactgaccacgccaccatggagaagccgcggggagtcaggtgcaccaatgggttctc
cgagcgggagctgccgcggcccggggccagcccgcctgccgagaagtcccggccgcccgaggccaagggc
gcacagccggccgacgcctggaaggcagggcggcaccgcagcgaggaggaaaaccaggtgaacctcccca
aactggcggctgcttactcgtccattctgctctcgctgggcgaggacccccagcggcaggggctgctcaa
gacgccctggagggcggccaccgccatgcagtacttcaccaagggataccaggagaccatctcagatgtc
ctgaatgatgctatatttgatgaagatcatgacgagatggtgattgtgaaggacatagatatgttctcca
tgtgtgagcatcaccttgttccatttgtaggaagggtccatattggctatcttcctaacaagcaagtcct
tggtctcagtaaacttgccaggattgtagaaatctacagtagacgactacaagttcaagagcgcctcacc
aaacagattgcggtggccatcacagaagccttgcagcctgctggcgttggagtagtgattgaagcgacac
acatgtgcatggtaatgcgaggcgtgcagaaaatgaacagcaagactgtcactagcaccatgctgggcgt
gttccgggaagaccccaagactcgggaggagttcctcacactaatcaggagctgaggccacctaatcaac
ctctggattacaaaatttgtgaaagattgactggtattcttaactatgttgctccttttacgctatgtgg
atacgctgctttaatgcctttgtatcatgctattgcttcccgtatggctttcattttctcctccttgtat
aaatcctggttgctgtctctttatgaggagttgtggcccgttgtcaggcaacgtggcgtggtgtgcactg
tgtttgctgacgcaacccccactggttggggcattgccaccacctgtcagctcctttccgggactttcgc
tttccccctccctattgccacggcggaactcatcgccgcctgccttgcccgctgctggacaggggctcgg
ctgttgggcactgacaattccgtggtgttgtcggggaaatcatcgtcctttcccatatgcagctcacaga
catgataagatacattgatgagtttggacaaaccacaactagaatgcagtgaaaaaaatgctttatttgt
gaaatttgtgatgctattgctttatttgtaaccattataagctgcaataaacaagttaacaacaacaatt
gcattcattttatgtttcaggttcagggggaggtgtgggaggttttttaaagcaagtaaaacctctacaa
atgtggtattggcccatctctatcggtatcgtagcataaccccttggggcctctaaacgggtcttgaggg
gttttttgtgcccctcgggccggattgctatctaccggcattggcgcagaaaaaaatgcctgatgcgacg
ctgcgcgtcttatactcccacatatgccagattcagcaacggatacggcttccccaacttgcccacttcc
atacgtgtcctccttaccagaaatttatccttaaggtcgtcagctatcctgcaggcgatctctcgatttc
gatcaagacattcctttaatggtcttttctggacaccactaggggtcagaagtagttcatcaaactttct
tccctccctaatctcattggttaccttgggctatcgaaacttaattaaccagtcaagtcagctacttggc
gagatcgacttgtctgggtttcgactacgctcagaattgcgtcagtcaagttcgatctggtccttgctat
tgcacccgttctccgattacgagtttcatttaaatcatgtgagcaaaaggccagcaaaaggccaggaacc
gtaaaaaggccgcgttgctggcgtttttccataggctccgcccccctgacgagcatcacaaaaatcgacg
ctcaagtcagaggtggcgaaacccgacaggactataaagataccaggcgtttccccctggaagctccctc
gtgcgctctcctgttccgaccctgccgcttaccggatacctgtccgcctttctcccttcgggaagcgtgg
cgctttctcatagctcacgctgtaggtatctcagttcggtgtaggtcgttcgctccaagctgggctgtgt
gcacgaaccccccgttcagcccgaccgctgcgccttatccggtaactatcgtcttgagtccaacccggta
agacacgacttatcgccactggcagcagccactggtaacaggattagcagagcgaggtatgtaggcggtg
ctacagagttcttgaagtggtggcctaactacggctacactagaagaacagtatttggtatctgcgctct
gctgaagccagttaccttcggaaaaagagttggtagctcttgatccggcaaacaaaccaccgctggtagc
ggtggtttttttgtttgcaagcagcagattacgcgcagaaaaaaaggatctcaagaagatcctttgatct
tttctacggggtctgacgctcagtggaacgaaaactcacgttaagggattttggtcatgagattatcaaa
aaggatcttcacctagatccttttaaattaaaaatgaagttttaaatcaatctaaagtatatatgagtaa
acttggtctgacagttaccaatgcttaatcagtgaggcacctatctcagcgatctgtctatttcgttcat
ccatagttgcatttaaatttccgaactctccaaggccctcgtcggaaaatcttcaaacctttcgtccgat
ccatcttgcaggctacctctcgaacgaactatcgcaagtctcttggccggccttgcgccttggctattgc
ttggcagcgcctatcgccaggtattactccaatcccgaatatccgagatcgggatcacccgagagaagtt
caacctacatcctcaatcccgatctatccgagatccgaggaatatcgaaatcggggcgcgcctggtgtac
cgagaacgatcctctcagtgcgagtctcgacgatccatatcgttgcttggcagtcagccagtcggaatcc
agcttgggacccaggaagtccaatcgtcagatattgtactcaagcctggtcacggcagcgtaccgatctg
tttaaacctagatattgatagtctgatcggtcaacgtataatcgagtcctagcttttgcaaacatctatc
aagagacaggatcagcaggaggctttcgcatgagtattcaacatttccgtgtcgcccttattcccttttt
tgcggcattttgccttcctgtttttgctcacccagaaacgctggtgaaagtaaaagatgctgaagatcag
ttgggtgcgcgagtgggttacatcgaactggatctcaacagcggtaagatccttgagagttttcgccccg
aagaacgctttccaatgatgagcacttttaaagttctgctatgtggcgcggtattatcccgtattgacgc
cgggcaagagcaactcggtcgccgcatacactattctcagaatgacttggttgagtattcaccagtcaca
gaaaagcatcttacggatggcatgacagtaagagaattatgcagtgctgccataaccatgagtgataaca
ctgcggccaacttacttctgacaacgattggaggaccgaaggagctaaccgcttttttgcacaacatggg
ggatcatgtaactcgccttgatcgttgggaaccggagctgaatgaagccataccaaacgacgagcgtgac
accacgatgcctgtagcaatggcaacaaccttgcgtaaactattaactggcgaactacttactctagctt
ccaggcaacagttgatagactggatggaggcggataaagttgcaggaccacttctgcgctcggcccttcc
ggctggctggtttattgctgataaatctggagccggtgagcgtgggtctcgcggtatcattgcagcactg
gggccagatggtaagccctcccgtatcgtagttatctacacgacggggagtcaggcaactatggatgaac
gaaatagacagatcgctgagataggtgcctcactgattaagcattggtaaccgattctaggtgcattggc
gcagaaaaaaatgcctgatgcgacgctgcgcgtcttatactcccacatatgccagattcagcaacggata
cggcttccccaacttgcccacttccatacgtgtcctccttaccagaaatttatccttaagatcccgaatc
gtttaaactcgactctggctctatcgaatctccgtcgtttcgagcttacgcgaacagccgtggcgctcat
ttgctcgtagggcatcgaatctcgtcagctatcgtcagcttacctttttggca

Preferably, the genetic construct comprises a nucleic acid sequence substantially as set out in SEQ ID No: 18, or a fragment or variant thereof.

One embodiment of the genetic construct is shown in FIG. 11, and is referred to herein as SEQ ID No: 19. This particular embodiment includes a CMV promoter and murine GCH1; these features would be easily replaceable by the skilled person for other variants as disclosed herein. The murine form of GCH1 is to facilitate preclinical testing of the construct. The murine form of GCH1 could be easily replaced by a skilled person, for instance the murine form could be replaced by the human form of GCH1.

[SEQ ID NO: 19]
ggcgatcgcggctcccgacatcttggaccattagctccacaggtatcttcttccctctagtggtcataac
agcagcttcagctacctctcaattcaaaaaacccctcaagacccgtttagaggccccaaggggttatgct
atcaatcgttgcgttacacacacaaaaaaccaacacacatccatcttcgatggatagcgattttattatc
taactgctgatcgagtgtagccagatctagtaatcaattacggggtcattagttcatagcccatatatgg
agttccgcgttacataacttacggtaaatggcccgcctggctgaccgcccaacgacccccgcccattgac
gtcaataatgacgtatgttcccatagtaacgccaatagggactttccattgacgtcaatgggtggagtat
ttacggtaaactgcccacttggcagtacatcaagtgtatcatatgccaagtacgccccctattgacgtca
atgacggtaaatggcccgcctggcattatgcccagtacatgaccttatgggactttcctacttggcagta
catctacgtattagtcatcgctattaccatgctgatgcggttttggcagtacatcaatgggcgtggatag
cggtttgactcacggggatttccaagtctccaccccattgacgtcaatgggagtttgttttggcaccaaa
atcaacgggactttccaaaatgtcgtaacaactccgccccattgacgcaaatgggcggtaggcgtgtacg
gtgggaggtctatataagcagagctggtttagtgaaccgtcagatcagatctagagatcccgggaccgcc
accatgagccccgcggggcccaaggtcccctggttcccaagaaaagtgtcagagctggacaagtgtcatc
acctggtcaccaagttcgaccctgacctggacttggaccacccgggcttctcggaccaggtgtaccgcca
gcgcaggaagctgattgctgagatcgccttccagtacaggcacggcgacccgattccccgtgtggagtac
accgccgaggagattgccacctggaaggaggtctacaccacgctgaagggcctctacgccacgcacgcct
gcggggagcacctggaggcctttgctttgctggagcgcttcagcggctaccgggaagacaatatccccca
gctggaggacgtctcccgcttcctgaaggagcgcacgggcttccagctgcggcctgtggccggcctgctg
tccgcccgggacttcctggccagcctggccttccgcgtgttccagtgcacccagtatatccgccacgcgt
cctcgcccatgcactcccctgagccggactgctgccacgagctgctggggcacgtgcccatgctggccga
ccgcaccttcgcgcagttctcgcaggacattggcctggcgtccctgggggcctcggatgaggaaattgag
aagctgtccacgctgtactggttcacggtggagttcgggctgtgtaagcagaacggggaggtgaaggcct
atggtgccgggctgctgtcctcctacggggagctcctgcactgcctgtctgaggagcctgagattcgggc
cttcgaccctgaggctgcggccgtgcagccctaccaagaccagacgtaccagtcagtctacttcgtgtct
gagagcttcagtgacgccaaggacaagctcaggagctatgcctcacgcatccagcgccccttctccgtga
agttcgacccgtacacgctggccatcgacgtgctggacagcccccaggccgtgcggcgctccctggaggg
tgtccaggatgagctggacacccttgcccatgcgctgagtgccattggctaacgcgcgaaacgcgcgccg
gtgaaacagaccctgaactttgatctgctgaaactggcgggcgatgtggaaagcaacccgggcccgatgg
agaagccgcggggagtcaggtgcaccaatgggttctccgagcgggagctgccgcggcccggggccagccc
gcctgccgagaagtcccggccgcccgaggccaagggcgcacagccggccgacgcctggaaggcagggcgg
caccgcagcgaggaggaaaaccaggtgaacctccccaaactggcggctgcttactcgtccattctgctct
cgctgggcgaggacccccagcggcaggggctgctcaagacgccctggagggcggccaccgccatgcagta
cttcaccaagggataccaggagaccatctcagatgtcctgaatgatgctatatttgatgaagatcatgac
gagatggtgattgtgaaggacatagatatgttctccatgtgtgagcatcaccttgttccatttgtaggaa
gggtccatattggctatcttcctaacaagcaagtccttggtctcagtaaacttgccaggattgtagaaat
ctacagtagacgactacaagttcaagagcgcctcaccaaacagattgcggtggccatcacagaagccttg
cagcctgctggcgttggagtagtgattgaagcgacacacatgtgcatggtaatgcgaggcgtgcagaaaa
tgaacagcaagactgtcactagcaccatgctgggcgtgttccgggaagaccccaagactcgggaggagtt
cctcacactaatcaggagctgaggccacctaatcaacctctggattacaaaatttgtgaaagattgactg
gtattcttaactatgttgctccttttacgctatgtggatacgctgctttaatgcctttgtatcatgctat
tgcttcccgtatggctttcattttctcctccttgtataaatcctggttgctgtctctttatgaggagttg
tggcccgttgtcaggcaacgtggcgtggtgtgcactgtgtttgctgacgcaacccccactggttggggca
ttgccaccacctgtcagctcctttccgggactttcgctttccccctccctattgccacggcggaactcat
cgccgcctgccttgcccgctgctggacaggggctcggctgttgggcactgacaattccgtggtgttgtcg
gggaaatcatcgtcctttcccatatgcagctcacagacatgataagatacattgatgagtttggacaaac
cacaactagaatgcagtgaaaaaaatgctttatttgtgaaatttgtgatgctattgctttatttgtaacc
attataagctgcaataaacaagttaacaacaacaattgcattcattttatgtttcaggttcagggggagg
tgtgggaggttttttaaagcaagtaaaacctctacaaatgtggtattggcccatctctatcggtatcgta
gcataaccccttggggcctctaaacgggtcttgaggggttttttgtgcccctcgggccggattgctatct
accggcattggcgcagaaaaaaatgcctgatgcgacgctgcgcgtcttatactcccacatatgccagatt
cagcaacggatacggcttccccaacttgcccacttccatacgtgtcctccttaccagaaatttatcctta
aggtcgtcagctatcctgcaggcgatctctcgatttcgatcaagacattcctttaatggtcttttctgga
caccactaggggtcagaagtagttcatcaaactttcttccctccctaatctcattggttaccttgggcta
tcgaaacttaattaaccagtcaagtcagctacttggcgagatcgacttgtctgggtttcgactacgctca
gaattgcgtcagtcaagttcgatctggtccttgctattgcacccgttctccgattacgagtttcatttaa
atcatgtgagcaaaaggccagcaaaaggccaggaaccgtaaaaaggccgcgttgctggcgtttttccata
ggctccgcccccctgacgagcatcacaaaaatcgacgctcaagtcagaggtggcgaaacccgacaggact
ataaagataccaggcgtttccccctggaagctccctcgtgcgctctcctgttccgaccctgccgcttacc
ggatacctgtccgcctttctcccttcgggaagcgtggcgctttctcatagctcacgctgtaggtatctca
gttcggtgtaggtcgttcgctccaagctgggctgtgtgcacgaaccccccgttcagcccgaccgctgcgc
cttatccggtaactatcgtcttgagtccaacccggtaagacacgacttatcgccactggcagcagccact
ggtaacaggattagcagagcgaggtatgtaggcggtgctacagagttcttgaagtggtggcctaactacg
gctacactagaagaacagtatttggtatctgcgctctgctgaagccagttaccttcggaaaaagagttgg
tagctcttgatccggcaaacaaaccaccgctggtagcggtggtttttttgtttgcaagcagcagattacg
cgcagaaaaaaaggatctcaagaagatcctttgatcttttctacggggtctgacgctcagtggaacgaaa
actcacgttaagggattttggtcatgagattatcaaaaaggatcttcacctagatccttttaaattaaaa
atgaagttttaaatcaatctaaagtatatatgagtaaacttggtctgacagttaccaatgcttaatcagt
gaggcacctatctcagcgatctgtctatttcgttcatccatagttgcatttaaatttccgaactctccaa
ggccctcgtcggaaaatcttcaaacctttcgtccgatccatcttgcaggctacctctcgaacgaactatc
gcaagtctcttggccggccttgcgccttggctattgcttggcagcgcctatcgccaggtattactccaat
cccgaatatccgagatcgggatcacccgagagaagttcaacctacatcctcaatcccgatctatccgaga
tccgaggaatatcgaaatcggggcgcgcctggtgtaccgagaacgatcctctcagtgcgagtctcgacga
tccatatcgttgcttggcagtcagccagtcggaatccagcttgggacccaggaagtccaatcgtcagata
ttgtactcaagcctggtcacggcagcgtaccgatctgtttaaacctagatattgatagtctgatcggtca
acgtataatcgagtcctagcttttgcaaacatctatcaagagacaggatcagcaggaggctttcgcatga
gtattcaacatttccgtgtcgcccttattcccttttttgcggcattttgccttcctgtttttgctcaccc
agaaacgctggtgaaagtaaaagatgctgaagatcagttgggtgcgcgagtgggttacatcgaactggat
ctcaacagcggtaagatccttgagagttttcgccccgaagaacgctttccaatgatgagcacttttaaag
ttctgctatgtggcgcggtattatcccgtattgacgccgggcaagagcaactcggtcgccgcatacacta
ttctcagaatgacttggttgagtattcaccagtcacagaaaagcatcttacggatggcatgacagtaaga
gaattatgcagtgctgccataaccatgagtgataacactgcggccaacttacttctgacaacgattggag
gaccgaaggagctaaccgcttttttgcacaacatgggggatcatgtaactcgccttgatcgttgggaacc
ggagctgaatgaagccataccaaacgacgagcgtgacaccacgatgcctgtagcaatggcaacaaccttg
cgtaaactattaactggcgaactacttactctagcttcccggcaacagttgatagactggatggaggcgg
ataaagttgcaggaccacttctgcgctcggcccttccggctggctggtttattgctgataaatctggagc
cggtgagcgtgggtctcgcggtatcattgcagcactggggccagatggtaagccctcccgtatcgtagtt
atctacacgacggggagtcaggcaactatggatgaacgaaatagacagatcgctgagataggtgcctcac
tgattaagcattggtaaccgattctaggtgcattggcgcagaaaaaaatgcctgatgcgacgctgcgcgt
cttatactcccacatatgccagattcagcaacggatacggcttccccaacttgcccacttccatacgtgt
cctccttaccagaaatttatccttaagatcccgaatcgtttaaactcgactctggctctatcgaatctcc
gtcgtttcgagcttacgcgaacagccgtggcgctcatttgctcgtcgggcatcgaatctcgtcagctatc
gtcagcttacctttttggca

Preferably, the genetic construct comprises a nucleic acid sequence substantially as set out in SEQ ID No: 19, or a fragment or variant thereof.

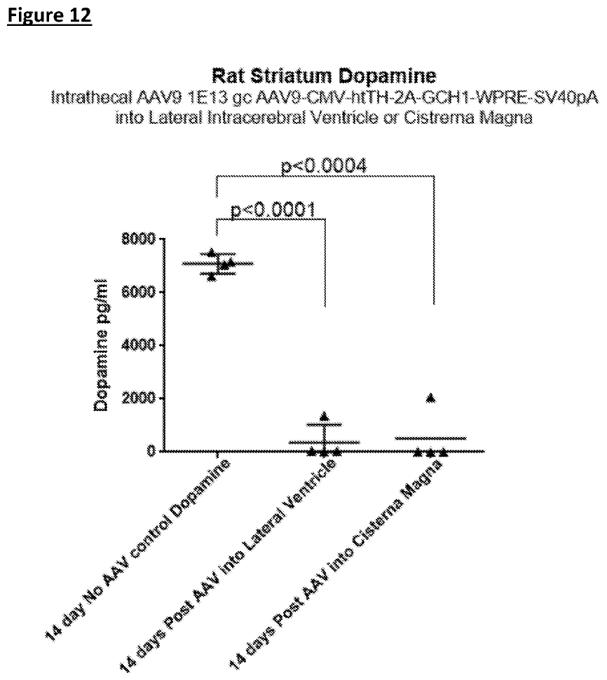

One embodiment of the genetic construct is shown in FIG. 12 and is referred to herein as SEQ ID No: 20. This particular embodiment includes a CMV promoter and murine GCH1; these features would be easily replaceable by the skilled person for other variants as disclosed herein. The murine form of GCH1 is to facilitate preclinical testing of the construct. The murine form of GCH1 could be easily replaced by a skilled person, for instance the murine form could be replaced by the human form of GCH1.

[SEQ ID NO: 20]
ggcgatcgcggctcccgacatcttggaccattagctccacaggtatcttcttccctctagtggtcataac
agcagcttcagctacctctcaattcaaaaaacccctcaagacccgtttagaggccccaaggggttatgct
atcaatcgttgcgttacacacacaaaaaaccaacacacatccatcttcgatggatagcgattttattatc
taactgctgatcgagtgtagccagatctagtaatcaattacggggtcattagttcatagcccatatatgg
agttccgcgttacataacttacggtaaatggcccgcctggctgaccgcccaacgacccccgcccattgac
gtcaataatgacgtatgttcccatagtaacgccaatagggactttccattgacgtcaatgggtggagtat
ttacggtaaactgcccacttggcagtacatcaagtgtatcatatgccaagtacgccccctattgacgtca
atgacggtaaatggcccgcctggcattatgcccagtacatgaccttatgggactttcctacttggcagta
catctacgtattagtcatcgctattaccatgctgatgcggttttggcagtacatcaatgggcgtggatag
cggtttgactcacggggatttccaagtctccaccccattgacgtcaatgggagtttgttttggcaccaaa
atcaacgggactttccaaaatgtcgtaacaactccgccccattgacgcaaatgggcggtaggcgtgtacg
gtgggaggtctatataagcagagctggtttagtgaaccgtcagatcagatctagagatcccgggaccgcc
accatgagccccgcggggcccaaggtcccctggttcccaagaaaagtgtcagagctggacaagtgtcatc
acctggtcaccaagttcgaccctgacctggacttggaccacccgggcttctcggaccaggtgtaccgcca
gcgcaggaagctgattgctgagatcgccttccagtacaggcacggcgacccgattccccgtgtggagtac
accgccgaggagattgccacctggaaggaggtctacaccacgctgaagggcctctacgccacgcacgcct
gcggggagcacctggaggcctttgctttgctggagcgcttcagcggctaccgggaagacaatatccccca
gctggaggacgtctcccgcttcctgaaggagcgcacgggcttccagctgcggcctgtggccggcctgctg
tccgcccgggacttcctggccagcctggccttccgcgtgttccagtgcacccagtatatccgccacgcgt
cctcgcccatgcactcccctgagccggactgctgccacgagctgctggggcacgtgcccatgctggccga
ccgcaccttcgcgcagttctcgcaggacattggcctggcgtccctgggggcctcggatgaggaaattgag
aagctgtccacgctgtactggttcacggtggagttcgggctgtgtaagcagaacggggaggtgaaggcct
atggtgccgggctgctgtcctcctacggggagctcctgcactgcctgtctgaggagcctgagattcgggc
cttcgaccctgaggctgcggccgtgcagccctaccaagaccagacgtaccagtcagtctacttcgtgtct
gagagcttcagtgacgccaaggacaagctcaggagctatgcctcacgcatccagcgccccttctccgtga
agttcgacccgtacacgctggccatcgacgtgctggacagcccccaggccgtgcggcgctccctggaggg
tgtccaggatgagctggacacccttgcccatgcgctgagtgccattggcggaggtggcgggtccgggggc
gggggtagcggtggcgggggctccgccaccatggagaagggccctgtgcgggcaccggcggagaagccgc
ggggcgccaggtgcagcaatgggttccccgagcgggatccgccgcggcccgggcccagcaggccggcgga
gaagcccccgcggcccgaggccaagagcgcgcagcccgcggacggctggaagggcgagcggccccgcagc
gaggaggataacgagctgaacctccctaacctggcagccgcctactcgtccatcctgagctcgctgggcg
agaacccccagcggcaagggctgctcaagacgccctggagggcggcctcggccatgcagttcttcaccaa
gggctaccaggagaccatctcagatgtcctaaacgatgctatatttgatgaagatcatgatgagatggtg
attgtgaaggacatagacatgttttccatgtgtgagcatcacttggttccatttgttggaaaggtccata
ttggttatcttcctaacaagcaagtccttggcctcagcaaacttgcgaggattgtagaaatctatagtag
aagactacaagttcaggagcgccttacaaaacaaattgctgtagcaatcacggaagccttgcggcctgct
ggagtcggggtagtggttgaagcaacacacatgtgtatggtaatgcgaggtgtacagaaaatgaacagca
aaactgtgaccagcacaatgttgggtgtgttccgggaggatccaaagactcgggaagagttcctgactct
cattaggagctgagccacctaatcaacctctggattacaaaatttgtgaaagattgactggtattcttaa
ctatgttgctccttttacgctatgtggatacgctgctttaatgcctttgtatcatgctattgcttcccgt
atggctttcattttctcctccttgtataaatcctggttgctgtctctttatgaggagttgtggcccgttg
tcaggcaacgtggcgtggtgtgcactgtgtttgctgacgcaacccccactggttggggcattgccaccac
ctgtcagctcctttccgggactttcgctttccccctccctattgccacggcggaactcatcgccgcctgc
cttgcccgctgctggacaggggctcggctgttgggcactgacaattccgtggtgttgtcggggaaatcat
cgtcctttcccatatgcagctcacagacatgataagatacattgatgagtttggacaaaccacaactaga
atgcagtgaaaaaaatgctttatttgtgaaatttgtgatgctattgctttatttgtaaccattataagct
gcaataaacaagttaacaacaacaattgcattcattttatgtttcaggttcagggggaggtgtgggaggt
tttttaaagcaagtaaaacctctacaaatgtggtattggcccatctctatcggtatcgtagcataacccc
ttggggcctctaaacgggtcttgaggggttttttgtgcccctcgggccggattgctatctaccggcattg
gcgcagaaaaaaatgcctgatgcgacgctgcgcgtcttatactcccacatatgccagattcagcaacgga
tacggcttccccaacttgcccacttccatacgtgtcctccttaccagaaatttatccttaaggtcgtcag
ctatcctgcaggcgatctctcgatttcgatcaagacattcctttaatggtcttttctggacaccactagg
ggtcagaagtagttcatcaaactttcttccctccctaatctcattggttaccttgggctatcgaaactta
attaaccagtcaagtcagctacttggcgagatcgacttgtctgggtttcgactacgctcagaattgcgtc
agtcaagttcgatctggtccttgctattgcacccgttctccgattacgagtttcatttaaatcatgtgag
caaaaggccagcaaaaggccaggaaccgtaaaaaggccgcgttgctggcgtttttccataggctccgccc
ccctgacgagcatcacaaaaatcgacgctcaagtcagaggtggcgaaacccgacaggactataaagatac
caggcgtttccccctggaagctccctcgtgcgctctcctgttccgaccctgccgcttaccggatacctgt
ccgcctttctcccttcgggaagcgtggcgctttctcatagctcacgctgtaggtatctcagttcggtgta
ggtcgttcgctccaagctgggctgtgtgcacgaaccccccgttcagcccgaccgctgcgccttatccggt
aactatcgtcttgagtccaacccggtaagacacgacttatcgccactggcagcagccactggtaacagga
ttagcagagcgaggtatgtaggcggtgctacagagttcttgaagtggtggcctaactacggctacactag
aagaacagtatttggtatctgcgctctgctgaagccagttaccttcggaaaaagagttggtagctcttga
tccggcaaacaaaccaccgctggtagcggtggtttttttgtttgcaagcagcagattacgcgcagaaaaa
aaggatctcaagaagatcctttgatcttttctacggggtctgacgctcagtggaacgaaaactcacgtta
agggattttggtcatgagattatcaaaaaggatcttcacctagatccttttaaattaaaaatgaagtttt
aaatcaatctaaagtatatatgagtaaacttggtctgacagttaccaatgcttaatcagtgaggcaccta
tctcagcgatctgtctatttcgttcatccatagttgcatttaaatttccgaactctccaaggccctcgtc
ggaaaatcttcaaacctttcgtccgatccatcttgcaggctacctctcgaacgaactatcgcaagtctct
tggccggccttgcgccttggctattgcttggcagcgcctatcgccaggtattactccaatcccgaatatc
cgagatcgggatcacccgagagaagttcaacctacatcctcaatcccgatctatccgagatccgaggaat
atcgaaatcggggcgcgcctggtgtaccgagaacgatcctctcagtgcgagtctcgacgatccatatcgt
tgcttggcagtcagccagtcggaatccagcttgggacccaggaagtccaatcgtcagatattgtactcaa
gcctggtcacggcagcgtaccgatctgtttaaacctagatattgatagtctgatcggtcaacgtataatc
gagtcctagcttttgcaaacatctatcaagagacaggatcagcaggaggctttcgcatgagtattcaaca
tttccgtgtcgcccttattcccttttttgcggcattttgccttcctgtttttgctcacccagaaacgctg
gtgaaagtaaaagatgctgaagatcagttgggtgcgcgagtgggttacatcgaactggatctcaacagcg
gtaagatccttgagagttttcgccccgaagaacgctttccaatgatgagcacttttaaagttctgctatg
tggcgcggtattatcccgtattgacgccgggcaagagcaactcggtcgccgcatacactattctcagaat
gacttggttgagtattcaccagtcacagaaaagcatcttacggatggcatgacagtaagagaattatgca
gtgctgccataaccatgagtgataacactgcggccaacttacttctgacaacgattggaggaccgaagga
gctaaccgcttttttgcacaacatgggggatcatgtaactcgccttgatcgttgggaaccggagctgaat
gaagccataccaaacgacgagcgtgacaccacgatgcctgtagcaatggcaacaaccttgcgtaaactat
taactggcgaactacttactctagcttcccggcaacagttgatagactggatggaggcggataaagttgc
aggaccacttctgcgctcggcccttccggctggctggtttattgctgataaatctggagccggtgagcgt
gggtctcgcggtatcattgcagcactggggccagatggtaagccctcccgtatcgtagttatctacacga
cggggagtcaggcaactatggatgaacgaaatagacagatcgctgagataggtgcctcactgattaagca
ttggtaaccgattctaggtgcattggcgcagaaaaaaatgcctgatgcgacgctgcgcgtcttatactcc
cacatatgccagattcagcaacggatacggcttccccaacttgcccacttccatacgtgtcctccttacc
agaaatttatccttaagatcccgaatcgtttaaactcgactctggctctatcgaatctccgtcgtttcga
gcttacgcgaacagccgtggcgctcatttgctcgtcgggcatcgaatctcgtcagctatcgtcagcttac
ctttttggca

Preferably, the genetic construct comprises a nucleic acid sequence substantially as set out in SEQ ID No: 20, or a fragment or variant thereof.

As described herein, by injecting the gene therapy construct of the first aspect into the intrathecal space, i.e. into the cerebrospinal fluid, it is surprisingly possible to raise the CSF level of L-DOPA (and dopamine), and use this as an novel and elegant route to impact L-DOPA and dopamine levels in the striatum of patients with Parkinson's disease. Additional advantages of using intrathecal injections are that it avoids the side effects experienced when using oral L-DOPA therapy, and also avoids the disadvantages of injecting directly into the striatal region of the patient's brain.

To this end, the inventors have created a series of recombinant expression vectors comprising the construct of the invention for use in treating Parkinson's disease.

Thus, according to a second aspect, there is provided a recombinant vector comprising the genetic construct, for use according to the first aspect.

As discussed under the first aspect, the inventors have found, surprisingly, that the construct does not need to be expressed in striatal cells, and thus the vector does not need to be targeted to striatal cells. Accordingly, preferably the vector does not comprise a modified capsid.

In one embodiment, the vector is configured to be targeted to cells of the ependyma and/or the adjacent tissue in the vicinity of the CSF.

The recombinant vector may be a recombinant AAV (rAAV) vector. The rAAV may be a naturally occurring vector or a vector with a hybrid AAV serotype. The rAAV may be AAV-1, AAV-2, AAV-3A, AAV-3B, AAV-4, AAV-5, AAV-6, AAV-7, AAV-8, AAV-9, AAV-10, and AAV-11. Preferably, the rAAV has tropism to neural tissue. In a preferred embodiment, the rAAV may be AAV1, AAV5, and more preferably AAV9.

The term “recombinant AAV (rAAV) vector” as used herein can mean a recombinant AAV-derived nucleic acid containing at least one terminal repeat sequence.

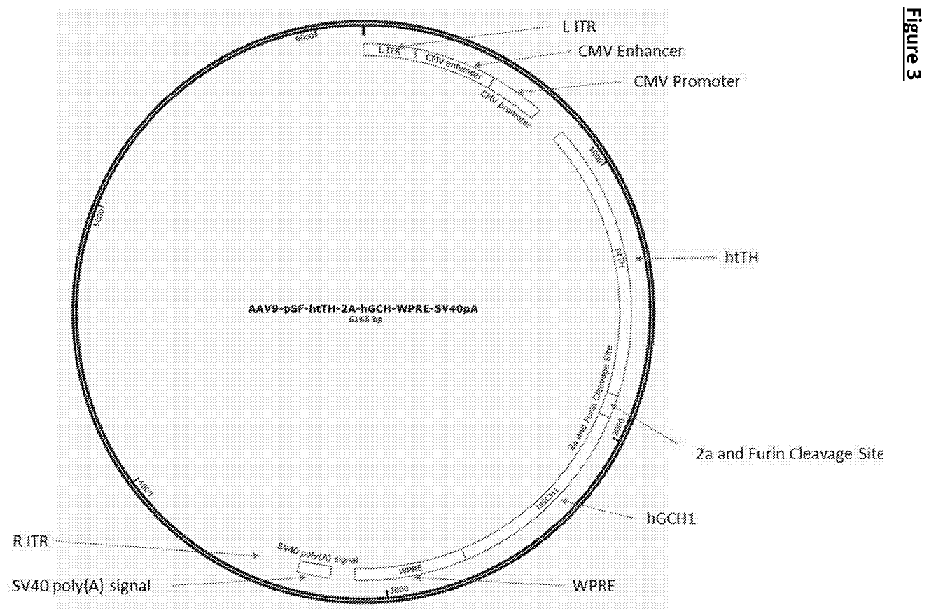

The following sequence, referred to herein as SEQ ID NO: 15, depicts a vector similar to SEQ ID NO: 13 depicted below, but this preferred embodiment includes a Furin cleavage site and a viral 2A peptide spacer, instead of the EMCV IRES. A map showing the features of a plasmid comprising SEQ ID NO: 15 is shown in FIG. 3.

[SEQ ID NO: 15]
CCTGCAGGCAGCTGCGCGCTCGCTCGCTCACTGAGGCCGCCCGGGCGTCGGGCGACCTTTGGTCGCCCGG
CCTCAGTGAGCGAGCGAGCGCGCAGAGAGGGAGTGGCCAACTCCATCACTAGGGGTTCCTGCGGCCGGTC
GCGTACTAGTAATCAATTACGGGGTCATTAGTTCATAGCCCATATATGGAGTTCCGCGTTACATAACTTA
CGGTAAATGGCCCGCCTGGCTGACCGCCCAACGACCCCCGCCCATTGACGTCAATAATGACGTATGTTCC
CATAGTAACGCCAATAGGGACTTTCCATTGACGTCAATGGGTGGAGTATTTACGGTAAACTGCCCACTTG
GCAGTACATCAAGTGTATCATATGCCAAGTACGCCCCCTATTGACGTCAATGACGGTAAATGGCCCGCCT
GGCATTATGCCCAGTACATGACCTTATGGGACTTTCCTACTTGGCAGTACATCTACGTATTAGTCATCGC
TATTACCATGCTGATGCGGTTTTGGCAGTACATCAATGGGCGTGGATAGCGGTTTGACTCACGGGGATTT
CCAAGTCTCCACCCCATTGACGTCAATGGGAGTTTGTTTTGGCACCAAAATCAACGGGACTTTCCAAAAT
GTCGTAACAACTCCGCCCCATTGACGCAAATGGGCGGTAGGCGTGTACGGTGGGAGGTCTATATAAGCAG
AGCTGGTTTAGTGAACCGTCAGATCAGATCTTTGTCGATCCTACCATCCACTCGACACACCCGCCAGCGG
CCGCTGCCAAGCTTCCGAGCTCTCGAATTCAAAGGAGGTACCCACCATGGCCACCATGAGCCCCGCGGGG
CCCAAGGTCCCCTGGTTCCCAAGAAAAGTGTCAGAGCTGGACAAGTGTCATCACCTGGTCACCAAGTTCG
ACCCTGACCTGGACTTGGACCACCCGGGCTTCTCGGACCAGGTGTACCGCCAGCGCAGGAAGCTGATTGC
TGAGATCGCCTTCCAGTACAGGCACGGCGACCCGATTCCCCGTGTGGAGTACACCGCCGAGGAGATTGCC
ACCTGGAAGGAGGTCTACACCACGCTGAAGGGCCTCTACGCCACGCACGCCTGCGGGGAGCACCTGGAGG
CCTTTGCTTTGCTGGAGCGCTTCAGCGGCTACCGGGAAGACAATATCCCCCAGCTGGAGGACGTCTCCCG
CTTCCTGAAGGAGCGCACGGGCTTCCAGCTGCGGCCTGTGGCCGGCCTGCTGTCCGCCCGGGACTTCCTG
GCCAGCCTGGCCTTCCGCGTGTTCCAGTGCACCCAGTATATCCGCCACGCGTCCTCGCCCATGCACTCCC
CTGAGCCGGACTGCTGCCACGAGCTGCTGGGGCACGTGCCCATGCTGGCCGACCGCACCTTCGCGCAGTT
CTCGCAGGACATTGGCCTGGCGTCCCTGGGGGCCTCGGATGAGGAAATTGAGAAGCTGTCCACGCTGTAC
TGGTTCACGGTGGAGTTCGGGCTGTGTAAGCAGAACGGGGAGGTGAAGGCCTATGGTGCCGGGCTGCTGT
CCTCCTACGGGGAGCTCCTGCACTGCCTGTCTGAGGAGCCTGAGATTCGGGCCTTCGACCCTGAGGCTGC
GGCCGTGCAGCCCTACCAAGACCAGACGTACCAGTCAGTCTACTTCGTGTCTGAGAGCTTCAGTGACGCC
AAGGACAAGCTCAGGAGCTATGCCTCACGCATCCAGCGCCCCTTCTCCGTGAAGTTCGACCCGTACACGC
TGGCCATCGACGTGCTGGACAGCCCCCAGGCCGTGCGGCGCTCCCTGGAGGGTGTCCAGGATGAGCTGGA
CACCCTTGCCCATGCGCTGAGTGCCATTGGCTAACGCGCGAAACGCGCGCCGGTGAAACAGACCCTGAAC
TTTGATCTGCTGAAACTGGCGGGCGATGTGGAAAGCAACCCGGGCCCGGCCACCATGGAGAAGGGCCCTG
TGCGGGCACCGGCGGAGAAGCCGCGGGGCGCCAGGTGCAGCAATGGGTTCCCCGAGCGGGATCCGCCGCG
GCCCGGGCCCAGCAGGCCGGCGGAGAAGCCCCCGCGGCCCGAGGCCAAGAGCGCGCAGCCCGCGGACGGC
TGGAAGGGCGAGCGGCCCCGCAGCGAGGAGGATAACGAGCTGAACCTCCCTAACCTGGCAGCCGCCTACT
CGTCCATCCTGAGCTCGCTGGGCGAGAACCCCCAGCGGCAAGGGCTGCTCAAGACGCCCTGGAGGGCGGC
CTCGGCCATGCAGTTCTTCACCAAGGGCTACCAGGAGACCATCTCAGATGTCCTAAACGATGCTATATTT
GATGAAGATCATGATGAGATGGTGATTGTGAAGGACATAGACATGTTTTCCATGTGTGAGCATCACTTGG
TTCCATTTGTTGGAAAGGTCCATATTGGTTATCTTCCTAACAAGCAAGTCCTTGGCCTCAGCAAACTTGC
GAGGATTGTAGAAATCTATAGTAGAAGACTACAAGTTCAGGAGCGCCTTACAAAACAAATTGCTGTAGCA
ATCACGGAAGCCTTGCGGCCTGCTGGAGTCGGGGTAGTGGTTGAAGCAACACACATGTGTATGGTAATGC
GAGGTGTACAGAAAATGAACAGCAAAACTGTGACCAGCACAATGTTGGGTGTGTTCCGGGAGGATCCAAA
GACTCGGGAAGAGTTCCTGACTCTCATTAGGAGCTGAGCCACCTAATCAACCTCTGGATTACAAAATTTG
TGAAAGATTGACTGGTATTCTTAACTATGTTGCTCCTTTTACGCTATGTGGATACGCTGCTTTAATGCCT
TTGTATCATGCTATTGCTTCCCGTATGGCTTTCATTTTCTCCTCCTTGTATAAATCCTGGTTGCTGTCTC
TTTATGAGGAGTTGTGGCCCGTTGTCAGGCAACGTGGCGTGGTGTGCACTGTGTTTGCTGACGCAACCCC
CACTGGTTGGGGCATTGCCACCACCTGTCAGCTCCTTTCCGGGACTTTCGCTTTCCCCCTCCCTATTGCC
ACGGCGGAACTCATCGCCGCCTGCCTTGCCCGCTGCTGGACAGGGGCTCGGCTGTTGGGCACTGACAATT
CCGTGGTGTTGTCGGGGAAATCATCGTCCTTTCCCTGGCTGACTGATACAATCGATTTCTGGATCCGCAG
GCCTCTGCTAGCTTGACTGACTGAGATACAGCGTACCTTCAGCTCACAGACATGATAAGATACATTGATG
AGTTTGGACAAACCACAACTAGAATGCAGTGAAAAAAATGCTTTATTTGTGAAATTTGTGATGCTATTGC
TTTATTTGTAACCATTATAAGCTGCAATAAACAAGTTAACAACAACAATTGCATTCATTTTATGTTTCAG
GTTCAGGGGGAGGTGTGGGAGGTTTTTTAAGCTTAACGCGGTAACCACGTGCGGACCCAACGGCCGCAGG
AACCCCTAGTGATGGAGTTGGCCACTCCCTCTCTGCGCGCTCGCTCGCTCACTGAGGCCGGGCGACCAAA
GGTCGCCCGACGCCCGGGCTTTGCCCGGGCGGCCTCAGTGAGCGAGCGAGCGCGCAGCTGCCTGCAGGGG
CGCCTGATGCGGTATTTTCTCCTTACGCATCTGTGCGGTATTTCACACCGCATACGTCAAAGCAACCATA
GTACGCGCCCTGTAGCGGCGCATTAAGCGCGGCGGGTGTGGTGGTTACGCGCAGCGTGACCGCTACACTT
GCCAGCGCCCTAGCGCCCGCTCCTTTCGCTTTCTTCCCTTCCTTTCTCGCCACGTTCGCCGGCTTTCCCC
GTCAAGCTCTAAATCGGGGGCTCCCTTTAGGGTTCCGATTTAGTGCTTTACGGCACCTCGACCCCAAAAA
ACTTGATTTGGGTGATGGTTCACGTAGTGGGCCATCGCCCTGATAGACGGTTTTTCGCCCTTTGACGTTG
GAGTCCACGTTCTTTAATAGTGGACTCTTGTTCCAAACTGGAACAACACTCAACCCTATCTCGGGCTATT
CTTTTGATTTATAAGGGATTTTGCCGATTTCGGCCTATTGGTTAAAAAATGAGCTGATTTAACAAAAATT
TAACGCGAATTTTAACAAAATATTAACGTTTACAATTTTATGGTGCACTCTCAGTACAATCTGCTCTGAT
GCCGCATAGTTAAGCCAGCCCCGACACCCGCCAACACCCGCTGACGCGCCCTGACGGGCTTGTCTGCTCC
CGGCATCCGCTTACAGACAAGCTGTGACCGTCTCCGGGAGCTGCATGTGTCAGAGGTTTTCACCGTCATC
ACCGAAACGCGCGAGACGAAAGGGCCTCGTGATACGCCTATTTTTATAGGTTAATGTCATGATAATAATG
GTTTCTTAGACGTCAGGTGGCACTTTTCGGGGAAATGTGCGCGGAACCCCTATTTGTTTATTTTTCTAAA
TACATTCAAATATGTATCCGCTCATGAGACAATAACCCTGATAAATGCTTCAATAATATTGAAAAAGGAA
GAGTATGAGTATTCAACATTTCCGTGTCGCCCTTATTCCCTTTTTTGCGGCATTTTGCCTTCCTGTTTTT
GCTCACCCAGAAACGCTGGTGAAAGTAAAAGATGCTGAAGATCAGTTGGGTGCACGAGTGGGTTACATCG
AACTGGATCTCAACAGCGGTAAGATCCTTGAGAGTTTTCGCCCCGAAGAACGTTTTCCAATGATGAGCAC
TTTTAAAGTTCTGCTATGTGGCGCGGTATTATCCCGTATTGACGCCGGGCAAGAGCAACTCGGTCGCCGC
ATACACTATTCTCAGAATGACTTGGTTGAGTACTCACCAGTCACAGAAAAGCATCTTACGGATGGCATGA
CAGTAAGAGAATTATGCAGTGCTGCCATAACCATGAGTGATAACACTGCGGCCAACTTACTTCTGACAAC
GATCGGAGGACCGAAGGAGCTAACCGCTTTTTTGCACAACATGGGGGATCATGTAACTCGCCTTGATCGT
TGGGAACCGGAGCTGAATGAAGCCATACCAAACGACGAGCGTGACACCACGATGCCTGTAGCAATGGCAA
CAACGTTGCGCAAACTATTAACTGGCGAACTACTTACTCTAGCTTCCCGGCAACAATTAATAGACTGGAT
GGAGGCGGATAAAGTTGCAGGACCACTTCTGCGCTCGGCCCTTCCGGCTGGCTGGTTTATTGCTGATAAA
TCTGGAGCCGGTGAGCGTGGGTCTCGCGGTATCATTGCAGCACTGGGGCCAGATGGTAAGCCCTCCCGTA
TCGTAGTTATCTACACGACGGGGAGTCAGGCAACTATGGATGAACGAAATAGACAGATCGCTGAGATAGG
TGCCTCACTGATTAAGCATTGGTAACTGTCAGACCAAGTTTACTCATATATACTTTAGATTGATTTAAAA
CTTCATTTTTAATTTAAAAGGATCTAGGTGAAGATCCTTTTTGATAATCTCATGACCAAAATCCCTTAAC
GTGAGTTTTCGTTCCACTGAGCGTCAGACCCCGTAGAAAAGATCAAAGGATCTTCTTGAGATCCTTTTTT
TCTGCGCGTAATCTGCTGCTTGCAAACAAAAAAACCACCGCTACCAGCGGTGGTTTGTTTGCCGGATCAA
GAGCTACCAACTCTTTTTCCGAAGGTAACTGGCTTCAGCAGAGCGCAGATACCAAATACTGTCCTTCTAG
TGTAGCCGTAGTTAGGCCACCACTTCAAGAACTCTGTAGCACCGCCTACATACCTCGCTCTGCTAATCCT
GTTACCAGTGGCTGCTGCCAGTGGCGATAAGTCGTGTCTTACCGGGTTGGACTCAAGACGATAGTTACCG
GATAAGGCGCAGCGGTCGGGCTGAACGGGGGGTTCGTGCACACAGCCCAGCTTGGAGCGAACGACCTACA
CCGAACTGAGATACCTACAGCGTGAGCTATGAGAAAGCGCCACGCTTCCCGAAGGGAGAAAGGCGGACAG
GTATCCGGTAAGCGGCAGGGTCGGAACAGGAGAGCGCACGAGGGAGCTTCCAGGGGGAAACGCCTGGTAT
CTTTATAGTCCTGTCGGGTTTCGCCACCTCTGACTTGAGCGTCGATTTTTGTGATGCTCGTCAGGGGGGC
GGAGCCTATGGAAAAACGCCAGCAACGCGGCCTTTTTACGGTTCCTGGCCTTTTGCTGGCCTTTTGCTCA
CATGT

Preferably, the vector comprises a nucleic acid sequence substantially as set out in SEQ ID No: 15, or a fragment or variant thereof.

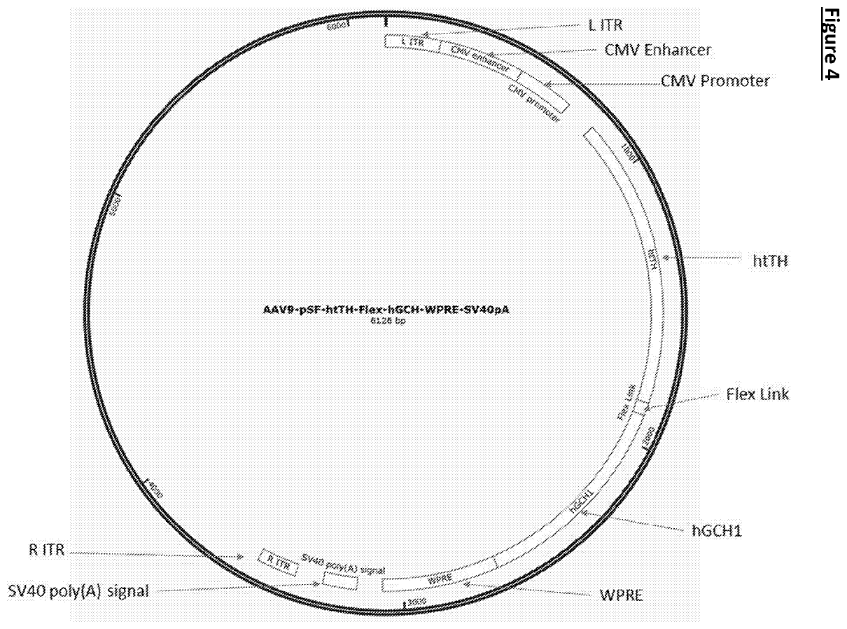

The following sequence, referred to herein as SEQ ID NO:16, depicts a vector similar to SEQ ID NO: 13, but this particular embodiment includes a flexible linker, instead of the EMCV IRES. A map showing the features of a plasmid comprising SEQ ID NO: 16 is shown in FIG. 4.

[SEQ ID NO: 16]
CCTGCAGGCAGCTGCGCGCTCGCTCGCTCACTGAGGCCGCCCGGGCGTCGGGCGACCTTTGGTCGCCCGG
CCTCAGTGAGCGAGCGAGCGCGCAGAGAGGGAGTGGCCAACTCCATCACTAGGGGTTCCTGCGGCCGGTC
GCGTACTAGTAATCAATTACGGGGTCATTAGTTCATAGCCCATATATGGAGTTCCGCGTTACATAACTTA
CGGTAAATGGCCCGCCTGGCTGACCGCCCAACGACCCCCGCCCATTGACGTCAATAATGACGTATGTTCC
CATAGTAACGCCAATAGGGACTTTCCATTGACGTCAATGGGTGGAGTATTTACGGTAAACTGCCCACTTG
GCAGTACATCAAGTGTATCATATGCCAAGTACGCCCCCTATTGACGTCAATGACGGTAAATGGCCCGCCT
GGCATTATGCCCAGTACATGACCTTATGGGACTTTCCTACTTGGCAGTACATCTACGTATTAGTCATCGC
TATTACCATGCTGATGCGGTTTTGGCAGTACATCAATGGGCGTGGATAGCGGTTTGACTCACGGGGATTT
CCAAGTCTCCACCCCATTGACGTCAATGGGAGTTTGTTTTGGCACCAAAATCAACGGGACTTTCCAAAAT
GTCGTAACAACTCCGCCCCATTGACGCAAATGGGCGGTAGGCGTGTACGGTGGGAGGTCTATATAAGCAG
AGCTGGTTTAGTGAACCGTCAGATCAGATCTTTGTCGATCCTACCATCCACTCGACACACCCGCCAGCGG
CCGCTGCCAAGCTTCCGAGCTCTCGAATTCAAAGGAGGTACCCACCATGGCCACCATGAGCCCCGCGGGG
CCCAAGGTCCCCTGGTTCCCAAGAAAAGTGTCAGAGCTGGACAAGTGTCATCACCTGGTCACCAAGTTCG
ACCCTGACCTGGACTTGGACCACCCGGGCTTCTCGGACCAGGTGTACCGCCAGCGCAGGAAGCTGATTGC
TGAGATCGCCTTCCAGTACAGGCACGGCGACCCGATTCCCCGTGTGGAGTACACCGCCGAGGAGATTGCC
ACCTGGAAGGAGGTCTACACCACGCTGAAGGGCCTCTACGCCACGCACGCCTGCGGGGAGCACCTGGAGG
CCTTTGCTTTGCTGGAGCGCTTCAGCGGCTACCGGGAAGACAATATCCCCCAGCTGGAGGACGTCTCCCG
CTTCCTGAAGGAGCGCACGGGCTTCCAGCTGCGGCCTGTGGCCGGCCTGCTGTCCGCCCGGGACTTCCTG
GCCAGCCTGGCCTTCCGCGTGTTCCAGTGCACCCAGTATATCCGCCACGCGTCCTCGCCCATGCACTCCC
CTGAGCCGGACTGCTGCCACGAGCTGCTGGGGCACGTGCCCATGCTGGCCGACCGCACCTTCGCGCAGTT
CTCGCAGGACATTGGCCTGGCGTCCCTGGGGGCCTCGGATGAGGAAATTGAGAAGCTGTCCACGCTGTAC
TGGTTCACGGTGGAGTTCGGGCTGTGTAAGCAGAACGGGGAGGTGAAGGCCTATGGTGCCGGGCTGCTGT
CCTCCTACGGGGAGCTCCTGCACTGCCTGTCTGAGGAGCCTGAGATTCGGGCCTTCGACCCTGAGGCTGC
GGCCGTGCAGCCCTACCAAGACCAGACGTACCAGTCAGTCTACTTCGTGTCTGAGAGCTTCAGTGACGCC
AAGGACAAGCTCAGGAGCTATGCCTCACGCATCCAGCGCCCCTTCTCCGTGAAGTTCGACCCGTACACGC
TGGCCATCGACGTGCTGGACAGCCCCCAGGCCGTGCGGCGCTCCCTGGAGGGTGTCCAGGATGAGCTGGA
CACCCTTGCCCATGCGCTGAGTGCCATTGGCTAAGGAGGTGGCGGGTCCGGGGGCGGGGGTAGCGGTGGC
GGGGGCTCCGCCACCATGGAGAAGGGCCCTGTGCGGGCACCGGCGGAGAAGCCGCGGGGCGCCAGGTGCA
GCAATGGGTTCCCCGAGCGGGATCCGCCGCGGCCCGGGCCCAGCAGGCCGGCGGAGAAGCCCCCGCGGCC
CGAGGCCAAGAGCGCGCAGCCCGCGGACGGCTGGAAGGGCGAGCGGCCCCGCAGCGAGGAGGATAACGAG
CTGAACCTCCCTAACCTGGCAGCCGCCTACTCGTCCATCCTGAGCTCGCTGGGCGAGAACCCCCAGCGGC
AAGGGCTGCTCAAGACGCCCTGGAGGGCGGCCTCGGCCATGCAGTTCTTCACCAAGGGCTACCAGGAGAC
CATCTCAGATGTCCTAAACGATGCTATATTTGATGAAGATCATGATGAGATGGTGATTGTGAAGGACATA
GACATGTTTTCCATGTGTGAGCATCACTTGGTTCCATTTGTTGGAAAGGTCCATATTGGTTATCTTCCTA
ACAAGCAAGTCCTTGGCCTCAGCAAACTTGCGAGGATTGTAGAAATCTATAGTAGAAGACTACAAGTTCA
GGAGCGCCTTACAAAACAAATTGCTGTAGCAATCACGGAAGCCTTGCGGCCTGCTGGAGTCGGGGTAGTG
GTTGAAGCAACACACATGTGTATGGTAATGCGAGGTGTACAGAAAATGAACAGCAAAACTGTGACCAGCA
CAATGTTGGGTGTGTTCCGGGAGGATCCAAAGACTCGGGAAGAGTTCCTGACTCTCATTAGGAGCTGAGC
CACCTAATCAACCTCTGGATTACAAAATTTGTGAAAGATTGACTGGTATTCTTAACTATGTTGCTCCTTT
TACGCTATGTGGATACGCTGCTTTAATGCCTTTGTATCATGCTATTGCTTCCCGTATGGCTTTCATTTTC
TCCTCCTTGTATAAATCCTGGTTGCTGTCTCTTTATGAGGAGTTGTGGCCCGTTGTCAGGCAACGTGGCG
TGGTGTGCACTGTGTTTGCTGACGCAACCCCCACTGGTTGGGGCATTGCCACCACCTGTCAGCTCCTTTC
CGGGACTTTCGCTTTCCCCCTCCCTATTGCCACGGCGGAACTCATCGCCGCCTGCCTTGCCCGCTGCTGG
ACAGGGGCTCGGCTGTTGGGCACTGACAATTCCGTGGTGTTGTCGGGGAAATCATCGTCCTTTCCCTGGC
TGACTGATACAATCGATTTCTGGATCCGCAGGCCTCTGCTAGCTTGACTGACTGAGATACAGCGTACCTT
CAGCTCACAGACATGATAAGATACATTGATGAGTTTGGACAAACCACAACTAGAATGCAGTGAAAAAAAT
GCTTTATTTGTGAAATTTGTGATGCTATTGCTTTATTTGTAACCATTATAAGCTGCAATAAACAAGTTAA
CAACAACAATTGCATTCATTTTATGTTTCAGGTTCAGGGGGAGGTGTGGGAGGTTTTTTAAGCTTAACGC
GGTAACCACGTGCGGACCCAACGGCCGCAGGAACCCCTAGTGATGGAGTTGGCCACTCCCTCTCTGCGCG
CTCGCTCGCTCACTGAGGCCGGGCGACCAAAGGTCGCCCGACGCCCGGGCTTTGCCCGGGCGGCCTCAGT
GAGCGAGCGAGCGCGCAGCTGCCTGCAGGGGCGCCTGATGCGGTATTTTCTCCTTACGCATCTGTGCGGT
ATTTCACACCGCATACGTCAAAGCAACCATAGTACGCGCCCTGTAGCGGCGCATTAAGCGCGGCGGGTGT
GGTGGTTACGCGCAGCGTGACCGCTACACTTGCCAGCGCCCTAGCGCCCGCTCCTTTCGCTTTCTTCCCT
TCCTTTCTCGCCACGTTCGCCGGCTTTCCCCGTCAAGCTCTAAATCGGGGGCTCCCTTTAGGGTTCCGAT
TTAGTGCTTTACGGCACCTCGACCCCAAAAAACTTGATTTGGGTGATGGTTCACGTAGTGGGCCATCGCC
CTGATAGACGGTTTTTCGCCCTTTGACGTTGGAGTCCACGTTCTTTAATAGTGGACTCTTGTTCCAAACT
GGAACAACACTCAACCCTATCTCGGGCTATTCTTTTGATTTATAAGGGATTTTGCCGATTTCGGCCTATT
GGTTAAAAAATGAGCTGATTTAACAAAAATTTAACGCGAATTTTAACAAAATATTAACGTTTACAATTTT
ATGGTGCACTCTCAGTACAATCTGCTCTGATGCCGCATAGTTAAGCCAGCCCCGACACCCGCCAACACCC
GCTGACGCGCCCTGACGGGCTTGTCTGCTCCCGGCATCCGCTTACAGACAAGCTGTGACCGTCTCCGGGA
GCTGCATGTGTCAGAGGTTTTCACCGTCATCACCGAAACGCGCGAGACGAAAGGGCCTCGTGATACGCCT
ATTTTTATAGGTTAATGTCATGATAATAATGGTTTCTTAGACGTCAGGTGGCACTTTTCGGGGAAATGTG
CGCGGAACCCCTATTTGTTTATTTTTCTAAATACATTCAAATATGTATCCGCTCATGAGACAATAACCCT
GATAAATGCTTCAATAATATTGAAAAAGGAAGAGTATGAGTATTCAACATTTCCGTGTCGCCCTTATTCC
CTTTTTTGCGGCATTTTGCCTTCCTGTTTTTGCTCACCCAGAAACGCTGGTGAAAGTAAAAGATGCTGAA
GATCAGTTGGGTGCACGAGTGGGTTACATCGAACTGGATCTCAACAGCGGTAAGATCCTTGAGAGTTTTC
GCCCCGAAGAACGTTTTCCAATGATGAGCACTTTTAAAGTTCTGCTATGTGGCGCGGTATTATCCCGTAT
TGACGCCGGGCAAGAGCAACTCGGTCGCCGCATACACTATTCTCAGAATGACTTGGTTGAGTACTCACCA
GTCACAGAAAAGCATCTTACGGATGGCATGACAGTAAGAGAATTATGCAGTGCTGCCATAACCATGAGTG
ATAACACTGCGGCCAACTTACTTCTGACAACGATCGGAGGACCGAAGGAGCTAACCGCTTTTTTGCACAA
CATGGGGGATCATGTAACTCGCCTTGATCGTTGGGAACCGGAGCTGAATGAAGCCATACCAAACGACGAG
CGTGACACCACGATGCCTGTAGCAATGGCAACAACGTTGCGCAAACTATTAACTGGCGAACTACTTACTC
TAGCTTCCCGGCAACAATTAATAGACTGGATGGAGGCGGATAAAGTTGCAGGACCACTTCTGCGCTCGGC
CCTTCCGGCTGGCTGGTTTATTGCTGATAAATCTGGAGCCGGTGAGCGTGGGTCTCGCGGTATCATTGCA
GCACTGGGGCCAGATGGTAAGCCCTCCCGTATCGTAGTTATCTACACGACGGGGAGTCAGGCAACTATGG
ATGAACGAAATAGACAGATCGCTGAGATAGGTGCCTCACTGATTAAGCATTGGTAACTGTCAGACCAAGT
TTACTCATATATACTTTAGATTGATTTAAAACTTCATTTTTAATTTAAAAGGATCTAGGTGAAGATCCTT
TTTGATAATCTCATGACCAAAATCCCTTAACGTGAGTTTTCGTTCCACTGAGCGTCAGACCCCGTAGAAA
AGATCAAAGGATCTTCTTGAGATCCTTTTTTTCTGCGCGTAATCTGCTGCTTGCAAACAAAAAAACCACC
GCTACCAGCGGTGGTTTGTTTGCCGGATCAAGAGCTACCAACTCTTTTTCCGAAGGTAACTGGCTTCAGC
AGAGCGCAGATACCAAATACTGTCCTTCTAGTGTAGCCGTAGTTAGGCCACCACTTCAAGAACTCTGTAG
CACCGCCTACATACCTCGCTCTGCTAATCCTGTTACCAGTGGCTGCTGCCAGTGGCGATAAGTCGTGTCT
TACCGGGTTGGACTCAAGACGATAGTTACCGGATAAGGCGCAGCGGTCGGGCTGAACGGGGGGTTCGTGC
ACACAGCCCAGCTTGGAGCGAACGACCTACACCGAACTGAGATACCTACAGCGTGAGCTATGAGAAAGCG
CCACGCTTCCCGAAGGGAGAAAGGCGGACAGGTATCCGGTAAGCGGCAGGGTCGGAACAGGAGAGCGCAC
GAGGGAGCTTCCAGGGGGAAACGCCTGGTATCTTTATAGTCCTGTCGGGTTTCGCCACCTCTGACTTGAG
CGTCGATTTTTGTGATGCTCGTCAGGGGGGCGGAGCCTATGGAAAAACGCCAGCAACGCGGCCTTTTTAC
GGTTCCTGGCCTTTTGCTGGCCTTTTGCTCACATGT

Preferably, the vector comprises a nucleic acid sequence substantially as set out in SEQ ID No: 16, or a fragment or variant thereof.

In an embodiment, the vector may be an AAV1 vector, comprising a human synapsin 1 promoter, a sequence encoding human truncated TH, an IRES, a sequence encoding human GCH1, a sequence encoding WPRE, a sequence encoding a poly A tail. The following sequence, referred to herein as SEQ ID NO: 13, depicts such a vector. This particular embodiment includes a CMV promoter, a CMV enhancer, an EMCV IRES, and a SV40 poly A tail. The individual features would be easily replaceable by the skilled person for alternatives as disclosed herein.

A map showing the features of a plasmid comprising SEQ ID NO: 13 is shown in FIG. 1.

[SEQ ID NO: 13]
CCTGCAGGCAGCTGCGCGCTCGCTCGCTCACTGAGGCCGCCCGGGCGTCGGGCGACCTTTGGTCGCCCGG
CCTCAGTGAGCGAGCGAGCGCGCAGAGAGGGAGTGGCCAACTCCATCACTAGGGGTTCCTGCGGCCGGTC
GCGTACTAGTAATCAATTACGGGGTCATTAGTTCATAGCCCATATATGGAGTTCCGCGTTACATAACTTA
CGGTAAATGGCCCGCCTGGCTGACCGCCCAACGACCCCCGCCCATTGACGTCAATAATGACGTATGTTCC
CATAGTAACGCCAATAGGGACTTTCCATTGACGTCAATGGGTGGAGTATTTACGGTAAACTGCCCACTTG
GCAGTACATCAAGTGTATCATATGCCAAGTACGCCCCCTATTGACGTCAATGACGGTAAATGGCCCGCCT
GGCATTATGCCCAGTACATGACCTTATGGGACTTTCCTACTTGGCAGTACATCTACGTATTAGTCATCGC
TATTACCATGCTGATGCGGTTTTGGCAGTACATCAATGGGCGTGGATAGCGGTTTGACTCACGGGGATTT
CCAAGTCTCCACCCCATTGACGTCAATGGGAGTTTGTTTTGGCACCAAAATCAACGGGACTTTCCAAAAT
GTCGTAACAACTCCGCCCCATTGACGCAAATGGGCGGTAGGCGTGTACGGTGGGAGGTCTATATAAGCAG
AGCTGGTTTAGTGAACCGTCAGATCAGATCTTTGTCGATCCTACCATCCACTCGACACACCCGCCAGCGG
CCGCTGCCAAGCTTCCGAGCTCTCGAATTCAAAGGAGGTACCCACCATGGCCACCATGAGCCCCGCGGGG
CCCAAGGTCCCCTGGTTCCCAAGAAAAGTGTCAGAGCTGGACAAGTGTCATCACCTGGTCACCAAGTTCG
ACCCTGACCTGGACTTGGACCACCCGGGCTTCTCGGACCAGGTGTACCGCCAGCGCAGGAAGCTGATTGC
TGAGATCGCCTTCCAGTACAGGCACGGCGACCCGATTCCCCGTGTGGAGTACACCGCCGAGGAGATTGCC
ACCTGGAAGGAGGTCTACACCACGCTGAAGGGCCTCTACGCCACGCACGCCTGCGGGGAGCACCTGGAGG
CCTTTGCTTTGCTGGAGCGCTTCAGCGGCTACCGGGAAGACAATATCCCCCAGCTGGAGGACGTCTCCCG
CTTCCTGAAGGAGCGCACGGGCTTCCAGCTGCGGCCTGTGGCCGGCCTGCTGTCCGCCCGGGACTTCCTG
GCCAGCCTGGCCTTCCGCGTGTTCCAGTGCACCCAGTATATCCGCCACGCGTCCTCGCCCATGCACTCCC
CTGAGCCGGACTGCTGCCACGAGCTGCTGGGGCACGTGCCCATGCTGGCCGACCGCACCTTCGCGCAGTT
CTCGCAGGACATTGGCCTGGCGTCCCTGGGGGCCTCGGATGAGGAAATTGAGAAGCTGTCCACGCTGTAC
TGGTTCACGGTGGAGTTCGGGCTGTGTAAGCAGAACGGGGAGGTGAAGGCCTATGGTGCCGGGCTGCTGT
CCTCCTACGGGGAGCTCCTGCACTGCCTGTCTGAGGAGCCTGAGATTCGGGCCTTCGACCCTGAGGCTGC
GGCCGTGCAGCCCTACCAAGACCAGACGTACCAGTCAGTCTACTTCGTGTCTGAGAGCTTCAGTGACGCC
AAGGACAAGCTCAGGAGCTATGCCTCACGCATCCAGCGCCCCTTCTCCGTGAAGTTCGACCCGTACACGC
TGGCCATCGACGTGCTGGACAGCCCCCAGGCCGTGCGGCGCTCCCTGGAGGGTGTCCAGGATGAGCTGGA
CACCCTTGCCCATGCGCTGAGTGCCATTGGCTAAACGTTACTGGCCGAAGCCGCTTGGAATAAGGCCGGT
GTGCGTTTGTCTATATGTTATTTTCCACCATATTGCCGTCTTTTGGCAATGTGAGGGCCCGGAAACCTGG
CCCTGTCTTCTTGACGAGCATTCCTAGGGGTCTTTCCCCTCTCGCCAAAGGAATGCAAGGTCTGTTGAAT
GTCGTGAAGGAAGCAGTTCCTCTGGAAGCTTCTTGAAGACAAACAACGTCTGTAGCGACCCTTTGCAGGC
AGCGGAACCCCCCACCTGGCGACAGGTGCCTCTGCGGCCAAAAGCCACGTGTATAAGATACACCTGCAAA
GGCGGCACAACCCCAGTGCCACGTTGTGAGTTGGATAGTTGTGGAAAGAGTCAAATGGCTCCCCTCAAGC
GTATTCAACAAGGGGCTGAAGGATGCCCAGAAGGTACCCCATTGTATGGGATCTGATCTGGGGCCTCGGT
GCACATGCTTTTCATGTGTTTAGTCGAGGTTAAAAAACGTCTAGGCCCCCCGAACCACGGGGACGTGGTT
TTCCTTTGAAAAACACGATGATAATAGCCACCATGGAGAAGGGCCCTGTGCGGGCACCGGCGGAGAAGCC
GCGGGGCGCCAGGTGCAGCAATGGGTTCCCCGAGCGGGATCCGCCGCGGCCCGGGCCCAGCAGGCCGGCG
GAGAAGCCCCCGCGGCCCGAGGCCAAGAGCGCGCAGCCCGCGGACGGCTGGAAGGGCGAGCGGCCCCGCA
GCGAGGAGGATAACGAGCTGAACCTCCCTAACCTGGCAGCCGCCTACTCGTCCATCCTGAGCTCGCTGGG
CGAGAACCCCCAGCGGCAAGGGCTGCTCAAGACGCCCTGGAGGGCGGCCTCGGCCATGCAGTTCTTCACC
AAGGGCTACCAGGAGACCATCTCAGATGTCCTAAACGATGCTATATTTGATGAAGATCATGATGAGATGG
TGATTGTGAAGGACATAGACATGTTTTCCATGTGTGAGCATCACTTGGTTCCATTTGTTGGAAAGGTCCA
TATTGGTTATCTTCCTAACAAGCAAGTCCTTGGCCTCAGCAAACTTGCGAGGATTGTAGAAATCTATAGT
AGAAGACTACAAGTTCAGGAGCGCCTTACAAAACAAATTGCTGTAGCAATCACGGAAGCCTTGCGGCCTG
CTGGAGTCGGGGTAGTGGTTGAAGCAACACACATGTGTATGGTAATGCGAGGTGTACAGAAAATGAACAG
CAAAACTGTGACCAGCACAATGTTGGGTGTGTTCCGGGAGGATCCAAAGACTCGGGAAGAGTTCCTGACT
CTCATTAGGAGCTGAGCCACCTAATCAACCTCTGGATTACAAAATTTGTGAAAGATTGACTGGTATTCTT
AACTATGTTGCTCCTTTTACGCTATGTGGATACGCTGCTTTAATGCCTTTGTATCATGCTATTGCTTCCC
GTATGGCTTTCATTTTCTCCTCCTTGTATAAATCCTGGTTGCTGTCTCTTTATGAGGAGTTGTGGCCCGT
TGTCAGGCAACGTGGCGTGGTGTGCACTGTGTTTGCTGACGCAACCCCCACTGGTTGGGGCATTGCCACC
ACCTGTCAGCTCCTTTCCGGGACTTTCGCTTTCCCCCTCCCTATTGCCACGGCGGAACTCATCGCCGCCT
GCCTTGCCCGCTGCTGGACAGGGGCTCGGCTGTTGGGCACTGACAATTCCGTGGTGTTGTCGGGGAAATC
ATCGTCCTTTCCCTGGCTGACTGATACAATCGATTTCTGGATCCGCAGGCCTCTGCTAGCTTGACTGACT
GAGATACAGCGTACCTTCAGCTCACAGACATGATAAGATACATTGATGAGTTTGGACAAACCACAACTAG
AATGCAGTGAAAAAAATGCTTTATTTGTGAAATTTGTGATGCTATTGCTTTATTTGTAACCATTATAAGC
TGCAATAAACAAGTTAACAACAACAATTGCATTCATTTTATGTTTCAGGTTCAGGGGGAGGTGTGGGAGG
TTTTTTAAGCTTAACGCGGTAACCACGTGCGGACCCAACGGCCGCAGGAACCCCTAGTGATGGAGTTGGC
CACTCCCTCTCTGCGCGCTCGCTCGCTCACTGAGGCCGGGCGACCAAAGGTCGCCCGACGCCCGGGCTTT
GCCCGGGCGGCCTCAGTGAGCGAGCGAGCGCGCAGCTGCCTGCAGGGGCGCCTGATGCGGTATTTTCTCC
TTACGCATCTGTGCGGTATTTCACACCGCATACGTCAAAGCAACCATAGTACGCGCCCTGTAGCGGCGCA
TTAAGCGCGGCGGGTGTGGTGGTTACGCGCAGCGTGACCGCTACACTTGCCAGCGCCCTAGCGCCCGCTC
CTTTCGCTTTCTTCCCTTCCTTTCTCGCCACGTTCGCCGGCTTTCCCCGTCAAGCTCTAAATCGGGGGCT
CCCTTTAGGGTTCCGATTTAGTGCTTTACGGCACCTCGACCCCAAAAAACTTGATTTGGGTGATGGTTCA
CGTAGTGGGCCATCGCCCTGATAGACGGTTTTTCGCCCTTTGACGTTGGAGTCCACGTTCTTTAATAGTG
GACTCTTGTTCCAAACTGGAACAACACTCAACCCTATCTCGGGCTATTCTTTTGATTTATAAGGGATTTT
GCCGATTTCGGCCTATTGGTTAAAAAATGAGCTGATTTAACAAAAATTTAACGCGAATTTTAACAAAATA
TTAACGTTTACAATTTTATGGTGCACTCTCAGTACAATCTGCTCTGATGCCGCATAGTTAAGCCAGCCCC
GACACCCGCCAACACCCGCTGACGCGCCCTGACGGGCTTGTCTGCTCCCGGCATCCGCTTACAGACAAGC
TGTGACCGTCTCCGGGAGCTGCATGTGTCAGAGGTTTTCACCGTCATCACCGAAACGCGCGAGACGAAAG
GGCCTCGTGATACGCCTATTTTTATAGGTTAATGTCATGATAATAATGGTTTCTTAGACGTCAGGTGGCA
CTTTTCGGGGAAATGTGCGCGGAACCCCTATTTGTTTATTTTTCTAAATACATTCAAATATGTATCCGCT
CATGAGACAATAACCCTGATAAATGCTTCAATAATATTGAAAAAGGAAGAGTATGAGTATTCAACATTTC
CGTGTCGCCCTTATTCCCTTTTTTGCGGCATTTTGCCTTCCTGTTTTTGCTCACCCAGAAACGCTGGTGA
AAGTAAAAGATGCTGAAGATCAGTTGGGTGCACGAGTGGGTTACATCGAACTGGATCTCAACAGCGGTAA
GATCCTTGAGAGTTTTCGCCCCGAAGAACGTTTTCCAATGATGAGCACTTTTAAAGTTCTGCTATGTGGC
GCGGTATTATCCCGTATTGACGCCGGGCAAGAGCAACTCGGTCGCCGCATACACTATTCTCAGAATGACT
TGGTTGAGTACTCACCAGTCACAGAAAAGCATCTTACGGATGGCATGACAGTAAGAGAATTATGCAGTGC
TGCCATAACCATGAGTGATAACACTGCGGCCAACTTACTTCTGACAACGATCGGAGGACCGAAGGAGCTA
ACCGCTTTTTTGCACAACATGGGGGATCATGTAACTCGCCTTGATCGTTGGGAACCGGAGCTGAATGAAG
CCATACCAAACGACGAGCGTGACACCACGATGCCTGTAGCAATGGCAACAACGTTGCGCAAACTATTAAC
TGGCGAACTACTTACTCTAGCTTCCCGGCAACAATTAATAGACTGGATGGAGGCGGATAAAGTTGCAGGA
CCACTTCTGCGCTCGGCCCTTCCGGCTGGCTGGTTTATTGCTGATAAATCTGGAGCCGGTGAGCGTGGGT
CTCGCGGTATCATTGCAGCACTGGGGCCAGATGGTAAGCCCTCCCGTATCGTAGTTATCTACACGACGGG
GAGTCAGGCAACTATGGATGAACGAAATAGACAGATCGCTGAGATAGGTGCCTCACTGATTAAGCATTGG
TAACTGTCAGACCAAGTTTACTCATATATACTTTAGATTGATTTAAAACTTCATTTTTAATTTAAAAGGA
TCTAGGTGAAGATCCTTTTTGATAATCTCATGACCAAAATCCCTTAACGTGAGTTTTCGTTCCACTGAGC
GTCAGACCCCGTAGAAAAGATCAAAGGATCTTCTTGAGATCCTTTTTTTCTGCGCGTAATCTGCTGCTTG
CAAACAAAAAAACCACCGCTACCAGCGGTGGTTTGTTTGCCGGATCAAGAGCTACCAACTCTTTTTCCGA
AGGTAACTGGCTTCAGCAGAGCGCAGATACCAAATACTGTCCTTCTAGTGTAGCCGTAGTTAGGCCACCA
CTTCAAGAACTCTGTAGCACCGCCTACATACCTCGCTCTGCTAATCCTGTTACCAGTGGCTGCTGCCAGT
GGCGATAAGTCGTGTCTTACCGGGTTGGACTCAAGACGATAGTTACCGGATAAGGCGCAGCGGTCGGGCT
GAACGGGGGGTTCGTGCACACAGCCCAGCTTGGAGCGAACGACCTACACCGAACTGAGATACCTACAGCG
TGAGCTATGAGAAAGCGCCACGCTTCCCGAAGGGAGAAAGGCGGACAGGTATCCGGTAAGCGGCAGGGTC
GGAACAGGAGAGCGCACGAGGGAGCTTCCAGGGGGAAACGCCTGGTATCTTTATAGTCCTGTCGGGTTTC
GCCACCTCTGACTTGAGCGTCGATTTTTGTGATGCTCGTCAGGGGGGCGGAGCCTATGGAAAAACGCCAG
CAACGCGGCCTTTTTACGGTTCCTGGCCTTTTGCTGGCCTTTTGCTCACATGT

Preferably, the vector comprises a nucleic acid sequence substantially as set out in SEQ ID No: 13, or a fragment or variant thereof.

The following sequence, referred to herein as SEQ ID NO: 14, depicts a vector similar to SEQ ID NO: 13, but this particular embodiment includes an FMDV IRES instead of the EMCV IRES. A map showing the features of a plasmid comprising SEQ ID NO: 14 is shown in FIG. 2.

[SEQ ID NO: 14]
CCTGCAGGCAGCTGCGCGCTCGCTCGCTCACTGAGGCCGCCCGGGCGTCGGGCGACCTTTGGTCGCCCGG
CCTCAGTGAGCGAGCGAGCGCGCAGAGAGGGAGTGGCCAACTCCATCACTAGGGGTTCCTGCGGCCGGTC
GCGTACTAGTAATCAATTACGGGGTCATTAGTTCATAGCCCATATATGGAGTTCCGCGTTACATAACTTA
CGGTAAATGGCCCGCCTGGCTGACCGCCCAACGACCCCCGCCCATTGACGTCAATAATGACGTATGTTCC
CATAGTAACGCCAATAGGGACTTTCCATTGACGTCAATGGGTGGAGTATTTACGGTAAACTGCCCACTTG
GCAGTACATCAAGTGTATCATATGCCAAGTACGCCCCCTATTGACGTCAATGACGGTAAATGGCCCGCCT
GGCATTATGCCCAGTACATGACCTTATGGGACTTTCCTACTTGGCAGTACATCTACGTATTAGTCATCGC
TATTACCATGCTGATGCGGTTTTGGCAGTACATCAATGGGCGTGGATAGCGGTTTGACTCACGGGGATTT
CCAAGTCTCCACCCCATTGACGTCAATGGGAGTTTGTTTTGGCACCAAAATCAACGGGACTTTCCAAAAT
GTCGTAACAACTCCGCCCCATTGACGCAAATGGGCGGTAGGCGTGTACGGTGGGAGGTCTATATAAGCAG
AGCTGGTTTAGTGAACCGTCAGATCAGATCTTTGTCGATCCTACCATCCACTCGACACACCCGCCAGCGG
CCGCTGCCAAGCTTCCGAGCTCTCGAATTCAAAGGAGGTACCCACCATGGCCACCATGAGCCCCGCGGGG
CCCAAGGTCCCCTGGTTCCCAAGAAAAGTGTCAGAGCTGGACAAGTGTCATCACCTGGTCACCAAGTTCG
ACCCTGACCTGGACTTGGACCACCCGGGCTTCTCGGACCAGGTGTACCGCCAGCGCAGGAAGCTGATTGC
TGAGATCGCCTTCCAGTACAGGCACGGCGACCCGATTCCCCGTGTGGAGTACACCGCCGAGGAGATTGCC
ACCTGGAAGGAGGTCTACACCACGCTGAAGGGCCTCTACGCCACGCACGCCTGCGGGGAGCACCTGGAGG
CCTTTGCTTTGCTGGAGCGCTTCAGCGGCTACCGGGAAGACAATATCCCCCAGCTGGAGGACGTCTCCCG
CTTCCTGAAGGAGCGCACGGGCTTCCAGCTGCGGCCTGTGGCCGGCCTGCTGTCCGCCCGGGACTTCCTG
GCCAGCCTGGCCTTCCGCGTGTTCCAGTGCACCCAGTATATCCGCCACGCGTCCTCGCCCATGCACTCCC
CTGAGCCGGACTGCTGCCACGAGCTGCTGGGGCACGTGCCCATGCTGGCCGACCGCACCTTCGCGCAGTT
CTCGCAGGACATTGGCCTGGCGTCCCTGGGGGCCTCGGATGAGGAAATTGAGAAGCTGTCCACGCTGTAC
TGGTTCACGGTGGAGTTCGGGCTGTGTAAGCAGAACGGGGAGGTGAAGGCCTATGGTGCCGGGCTGCTGT
CCTCCTACGGGGAGCTCCTGCACTGCCTGTCTGAGGAGCCTGAGATTCGGGCCTTCGACCCTGAGGCTGC
GGCCGTGCAGCCCTACCAAGACCAGACGTACCAGTCAGTCTACTTCGTGTCTGAGAGCTTCAGTGACGCC
AAGGACAAGCTCAGGAGCTATGCCTCACGCATCCAGCGCCCCTTCTCCGTGAAGTTCGACCCGTACACGC
TGGCCATCGACGTGCTGGACAGCCCCCAGGCCGTGCGGCGCTCCCTGGAGGGTGTCCAGGATGAGCTGGA
CACCCTTGCCCATGCGCTGAGTGCCATTGGCTAAAGCAGGTTTCCCCAACTGACACAAAACGTGCAACTT
GAAACTCCGCCTGGTCTTTCCAGGTCTAGAGGGGTAACACTTTGTACTGCGTTTGGCTCCACGCTCGATC
CACTGGCGAGTGTTAGTAACAGCACTGTTGCTTCGTAGCGGAGCATGACGGCCGTGGGAACTCCTCCTTG
GTAACAAGGACCCACGGGGCCAAAAGCCACGCCCACACGGGCCCGTCATGTGTGCAACCCCAGCACGGCG
ACTTTACTGCGAAACCCACTTTAAAGTGACATTGAAACTGGTACCCACACACTGGTGACAGGCTAAGGAT
GCCCTTCAGGTACCCCGAGGTAACACGCGACACTCGGGATCTGAGAAGGGGACTGGGGCTTCTATAAAAG
CGCTCGGTTTAAAAAGCTTCTATGCCTGAATAGGTGACCGGAGGTCGGCACCTTTCCTTTGCAATTACTG
ACCACGCCACCATGGAGAAGGGCCCTGTGCGGGCACCGGCGGAGAAGCCGCGGGGCGCCAGGTGCAGCAA
TGGGTTCCCCGAGCGGGATCCGCCGCGGCCCGGGCCCAGCAGGCCGGCGGAGAAGCCCCCGCGGCCCGAG
GCCAAGAGCGCGCAGCCCGCGGACGGCTGGAAGGGCGAGCGGCCCCGCAGCGAGGAGGATAACGAGCTGA
ACCTCCCTAACCTGGCAGCCGCCTACTCGTCCATCCTGAGCTCGCTGGGCGAGAACCCCCAGCGGCAAGG
GCTGCTCAAGACGCCCTGGAGGGCGGCCTCGGCCATGCAGTTCTTCACCAAGGGCTACCAGGAGACCATC
TCAGATGTCCTAAACGATGCTATATTTGATGAAGATCATGATGAGATGGTGATTGTGAAGGACATAGACA
TGTTTTCCATGTGTGAGCATCACTTGGTTCCATTTGTTGGAAAGGTCCATATTGGTTATCTTCCTAACAA
GCAAGTCCTTGGCCTCAGCAAACTTGCGAGGATTGTAGAAATCTATAGTAGAAGACTACAAGTTCAGGAG
CGCCTTACAAAACAAATTGCTGTAGCAATCACGGAAGCCTTGCGGCCTGCTGGAGTCGGGGTAGTGGTTG
AAGCAACACACATGTGTATGGTAATGCGAGGTGTACAGAAAATGAACAGCAAAACTGTGACCAGCACAAT
GTTGGGTGTGTTCCGGGAGGATCCAAAGACTCGGGAAGAGTTCCTGACTCTCATTAGGAGCTGAGCCACC
TAATCAACCTCTGGATTACAAAATTTGTGAAAGATTGACTGGTATTCTTAACTATGTTGCTCCTTTTACG
CTATGTGGATACGCTGCTTTAATGCCTTTGTATCATGCTATTGCTTCCCGTATGGCTTTCATTTTCTCCT
CCTTGTATAAATCCTGGTTGCTGTCTCTTTATGAGGAGTTGTGGCCCGTTGTCAGGCAACGTGGCGTGGT
GTGCACTGTGTTTGCTGACGCAACCCCCACTGGTTGGGGCATTGCCACCACCTGTCAGCTCCTTTCCGGG
ACTTTCGCTTTCCCCCTCCCTATTGCCACGGCGGAACTCATCGCCGCCTGCCTTGCCCGCTGCTGGACAG
GGGCTCGGCTGTTGGGCACTGACAATTCCGTGGTGTTGTCGGGGAAATCATCGTCCTTTCCCTGGCTGAC
TGATACAATCGATTTCTGGATCCGCAGGCCTCTGCTAGCTTGACTGACTGAGATACAGCGTACCTTCAGC
TCACAGACATGATAAGATACATTGATGAGTTTGGACAAACCACAACTAGAATGCAGTGAAAAAAATGCTT
TATTTGTGAAATTTGTGATGCTATTGCTTTATTTGTAACCATTATAAGCTGCAATAAACAAGTTAACAAC
AACAATTGCATTCATTTTATGTTTCAGGTTCAGGGGGAGGTGTGGGAGGTTTTTTAAGCTTAACGCGGTA
ACCACGTGCGGACCCAACGGCCGCAGGAACCCCTAGTGATGGAGTTGGCCACTCCCTCTCTGCGCGCTCG
CTCGCTCACTGAGGCCGGGCGACCAAAGGTCGCCCGACGCCCGGGCTTTGCCCGGGCGGCCTCAGTGAGC
GAGCGAGCGCGCAGCTGCCTGCAGGGGCGCCTGATGCGGTATTTTCTCCTTACGCATCTGTGCGGTATTT
CACACCGCATACGTCAAAGCAACCATAGTACGCGCCCTGTAGCGGCGCATTAAGCGCCGCGGGTGTGGTG
GTTACGCGCAGCGTGACCGCTACACTTGCCAGCGCCCTAGCGCCCGCTCCTTTCGCTTTCTTCCCTTCCT
TTCTCGCCACGTTCGCCGGCTTTCCCCGTCAAGCTCTAAATCGGGGGCTCCCTTTAGGGTTCCGATTTAG
TGCTTTACGGCACCTCGACCCCAAAAAACTTGATTTGGGTGATGGTTCACGTAGTGGGCCATCGCCCTGA
TAGACGGTTTTTCGCCCTTTGACGTTGGAGTCCACGTTCTTTAATAGTGGACTCTTGTTCCAAACTGGAA
CAACACTCAACCCTATCTCGGGCTATTCTTTTGATTTATAAGGGATTTTGCCGATTTCGGCCTATTGGTT
AAAAAATGAGCTGATTTAACAAAAATTTAACGCGAATTTTAACAAAATATTAACGTTTACAATTTTATGG
TGCACTCTCAGTACAATCTGCTCTGATGCCGCATAGTTAAGCCAGCCCCGACACCCGCCAACACCCGCTG
ACGCGCCCTGACGGGCTTGTCTGCTCCCGGCATCCGCTTACAGACAAGCTGTGACCGTCTCCGGGAGCTG
CATGTGTCAGAGGTTTTCACCGTCATCACCGAAACGCGCGAGACGAAAGGGCCTCGTGATACGCCTATTT
TTATAGGTTAATGTCATGATAATAATGGTTTCTTAGACGTCAGGTGGCACTTTTCGGGGAAATGTGCGCG
GAACCCCTATTTGTTTATTTTTCTAAATACATTCAAATATGTATCCGCTCATGAGACAATAACCCTGATA
AATGCTTCAATAATATTGAAAAAGGAAGAGTATGAGTATTCAACATTTCCGTGTCGCCCTTATTCCCTTT
TTTGCGGCATTTTGCCTTCCTGTTTTTGCTCACCCAGAAACGCTGGTGAAAGTAAAAGATGCTGAAGATC
AGTTGGGTGCACGAGTGGGTTACATCGAACTGGATCTCAACAGCGGTAAGATCCTTGAGAGTTTTCGCCC
CGAAGAACGTTTTCCAATGATGAGCACTTTTAAAGTTCTGCTATGTGGCGCGGTATTATCCCGTATTGAC
GCCGGGCAAGAGCAACTCGGTCGCCGCATACACTATTCTCAGAATGACTTGGTTGAGTACTCACCAGTCA
CAGAAAAGCATCTTACGGATGGCATGACAGTAAGAGAATTATGCAGTGCTGCCATAACCATGAGTGATAA
CACTGCGGCCAACTTACTTCTGACAACGATCGGAGGACCGAAGGAGCTAACCGCTTTTTTGCACAACATG
GGGGATCATGTAACTCGCCTTGATCGTTGGGAACCGGAGCTGAATGAAGCCATACCAAACGACGAGCGTG
ACACCACGATGCCTGTAGCAATGGCAACAACGTTGCGCAAACTATTAACTGGCGAACTACTTACTCTAGC
TTCCCGGCAACAATTAATAGACTGGATGGAGGCGGATAAAGTTGCAGGACCACTTCTCCGCTCGGCCCTT
CCGGCTGGCTGGTTTATTGCTGATAAATCTGGAGCCGGTGAGCGTGGGTCTCGCGGTATCATTGCAGCAC
TGGGGCCAGATGGTAAGCCCTCCCGTATCGTAGTTATCTACACGACGGGGAGTCAGGCAACTATGGATGA
ACGAAATAGACAGATCGCTGAGATAGGTGCCTCACTGATTAAGCATTGGTAACTGTCAGACCAAGTTTAC
TCATATATACTTTAGATTGATTTAAAACTTCATTTTTAATTTAAAAGGATCTAGGTGAAGATCCTTTTTG
ATAATCTCATGACCAAAATCCCTTAACGTGAGTTTTCGTTCCACTGAGCGTCAGACCCCGTAGAAAAGAT
CAAAGGATCTTCTTGAGATCCTTTTTTTCTGCGCGTAATCTGCTGCTTGCAAACAAAAAAACCACCGCTA
CCAGCGGTGGTTTGTTTGCCGGATCAAGAGCTACCAACTCTTTTTCCGAAGGTAACTGGCTTCAGCAGAG
CGCAGATACCAAATACTGTCCTTCTAGTGTAGCCGTAGTTAGGCCACCACTTCAAGAACTCTGTAGCACC
GCCTACATACCTCGCTCTGCTAATCCTGTTACCAGTGGCTGCTGCCAGTGGCGATAAGTCGTGTCTTACC
GGGTTGGACTCAAGACGATAGTTACCGGATAAGGCGCAGCGGTCGGGCTGAACGGGGGGTTCGTGCACAC
AGCCCAGCTTGGAGCGAACGACCTACACCGAACTGAGATACCTACAGCGTGAGCTATGAGAAAGCGCCAC
GCTTCCCGAAGGGAGAAAGGCGGACAGGTATCCGGTAAGCGGCAGGGTCGGAACAGGAGAGCGCACGAGG
GAGCTTCCAGGGGGAAACGCCTGGTATCTTTATAGTCCTGTCGGGTTTCGCCACCTCTGACTTGAGCGTC
GATTTTTGTGATGCTCGTCAGGGGGGCGGAGCCTATGGAAAAACGCCAGCAACGCGGCCTTTTTACGGTT
CCTGGCCTTTTGCTGGCCTTTTGCTCACATGT

Preferably, the vector comprises a nucleic acid sequence substantially as set out in SEQ ID No: 14, or a fragment or variant thereof.

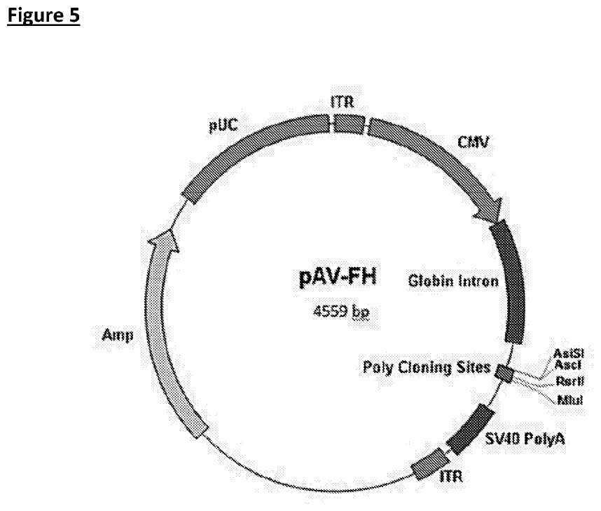

The following sequence, referred to herein as SEQ ID NO: 17, encodes a vector carrying AAV2 right and left ITRs. This vector is suitable for the production of AAV vectors; the genetic constructs of the first aspect can be subcloned into this vector. A map showing the features of a plasmid comprising SEQ ID NO: 17 is shown in FIG. 5. This vector is purely for illustrative purposes, and the skilled person would be aware of other suitable vectors. The pAV-FH vector sequence shown in FIG. 5, and other suitable vectors for the production of AAV vectors, are commercially available.

[SEQ ID NO: 17]
CCTGCAGGCAGCTGCGCGCTCGCTCGCTCACTGAGGCCGCCCGGGCGTCGGGCGACCTTTGGTCGCCCGG
CCTCAGTGAGCGAGCGAGCGCGCAGAGAGGGAGTGGCCAACTCCATCACTAGGGGTTCCTGCGGCCGGTC
GCGTCTAGTTATTAATAGTAATCAATTACGGGGTCATTAGTTCATAGCCCATATATGGAGTTCCGCGTTA
CATAACTTACGGTAAATGGCCCGCCTGGCTGACCGCCCAACGACCCCCGCCCATTGACGTCAATAATGAC
GTATGTTCCCATAGTAACGCAATAGGGACTTTCCATTGACGTCAATGGGTGGAGTATTTACGGTAAACTG
CCCACTTGGCAGTACATCAAGTGTATCATATGCCAAGTACGCCCCCTATTGACGTCAATGACGGTAAATG
GCCCGCCTGGCATTATGCCCAGTACATGACCTTATGGGACTTTCCTACTTGGCAGTACATCTACGTATTA
GTCATCGCTATTACCATGGTGATGCGGTTTTGGCAGTACATCAATGGGCGTGGATAGCGGTTTGACTCAC
GGGGATTTCCAAGTCTCCACCCCATTGACGTCAATGGGAGTTTGTTTTGCACCAAAATCAACGGGACTTT
CCAAAATGTCGTAACAACTCCGCCCCATTGACGCAAATGGGCGGTAGGCGTGTACGGTGGGAGGTCTATA
TAAGCAGAGCTGTTTAGTGAACCGTCAGATCGCCTGGAGACGCCATCCACGCTGTTTTGACCTCCATAGA
AGACACCGGGACCGATCCAGCCTCCGCGGATTCGAATCCCGGCCGGGAACGGTGCATTGGAACGCGGATT
CCCCGTGCCAAGAGTGACGTAAGTACCGCCTATAGAGTCTATAGGCCCACAAAAAATGCTTTCTTCTTTT
AATATACTTTTTTGTTTATCTTATTTCTAATACTTTCCCTAATCTCTTTCTTTCAGGGCAATAATGATAC
AATGTATCATGCCTCTTTGCACCATTCTAAAGAATAACAGTGATAATTTCTGGGTTAAGGCAATAGCAAT
ATTTCTGCATATAAATATTTCTGCATATAAATTGTAACTGATGTAAGAGGTTTCATATTGCTAATAGCAG
CTACAATCCAGCTACCATTCTGCTTTTATTTTATGGTTGGGATAAGGCTGGATTATTCTGAGTCCAAGCT
AGGCCCTTTTGCTAATCATGTTCATACCTCTTATCTTCCTCCCACAGCTCCTGGGCAACGTGCTGGTCTG
TGTGCTGGCCCATCACTTTGGCAAAGAATTGGGATTCGAACATCGATTGAATTCAGATCCGCTAGTAATA
CGACTCACTATAGGGAGAGGATCCGGTACCGAGGAGATCTGCCGCCGCGATCGCCGGCGCGCCAGATCTC
ACGCTTAACTAGCTAGCGGACCGACGCGTACGCGGCCGCTCGAGGATTATAAGGATGACGACGATAAATT
CGTCGAGCACCACCACCACCACCACTAATAAGGTTTATCCGATCCACCGGATCTAGATAAGATATCCGAT
CCACCGGATCTAGATAACTGATCATAATCAGCCATACCACATTTGTAGAGGTTTTACTTGCTTTAAAAAA
CCTCCCACACCTCCCCCTGAACCTGAAACATAAAATGAATGCAATTGTTGTTGTTAACTTGTTTATTGCA
GCTTATAATGGTTACAAATAAAGCAATAGCATCACAAATTTCACAAATAAAGCATTTTTTTCACTGCATT
CTAGTTGTGGTTTGTCCAAACTCATCAATGTATCTTAACGCGGTAACCACGTGCGGACCCAACGGCCGCA
GGAACCCCTAGTGATGGAGTTGGCCACTCCCTCTagagCTGCGCGCTCGCTCGCTCACTGAGGCCGGGCG
ACCAAAGGTCGCCCGACGCCCGGGCTTTGCCCGGGCGGCCTCAGTGAGCGAGCGAGCGCGCAGCTGCCTG
CAGGGGCGCCTGATGCGGTATTTTCTCCTTACGCATCTGTGCGGTATTTCACACCGCATACGTCAAAGCA
ACCATAGTACGCGCCCTGTAGCGGCGCATTAAGCGCGGCGGGTGTGGTGGTTACGCGCAGCGTGACCGCT
ACACTTGCCAGCGCCCTAGCGCCCGCTCCTTTCGCTTTCTTCCCTTCCTTTCTCGCCACGTTCGCCGGCT
TTCCCCGTCAAGCTCTAAATCGGGGGCTCCCTTTAGGGTTCCGATTTAGTGCTTTACGGCACCTCGACCC
CAAAAAACTTGATTTGGGTGATGGTTCACGTAGTGGGCCATCGCCCTGATAGACGGTTTTTCGCCCTTTG
ACGTTGGAGTCCACGTTCTTTAATAGTGGACTCTTGTTCCAAACTGGAACAACACTCAACCCTATCTCGG
GCTATTCTTTTGATTTATAAGGGATTTTGCCGATTTCGGCCTATTGGTTAAAAAATGAGCTGATTTAACA
AAAATTTAACGCGAATTTTAACAAAATATTAACGTTTACAATTTTATGGTGCACTCTCAGTACAATCTGC
TCTGATGCCGCATAGTTAAGCCAGCCCCGACACCCGCCAACACCCGCTGACGCGCCCTGACGGGCTTGTC
TGCTCCCGGCATCCGCTTACAGACAAGCTGTGACCGTCTCCGGGAGCTGCATGTGTCAGAGGTTTTCACC
GTCATCACCGAAACGCGCGAGACGAAAGGGCCTCGTGATACGCCTATTTTTATAGGTTAATGTCATGATA
ATAATGGTTTCTTAGACGTCAGGTGGCACTTTTCGGGGAAATGTGCGCGGAACCCCTATTTGTTTATTTT
TCTAAATACATTCAAATATGTATCCGCTCATGAGACAATAACCCTGATAAATGCTTCAATAATATTGAAA
AAGGAAGAGTATGAGTATTCAACATTTCCGTGTCGCCCTTATTCCCTTTTTTGCGGCATTTTGCCTTCCT
GTTTTTGCTCACCCAGAAACGCTGGTGAAAGTAAAAGATGCTGAAGATCAGTTGGGTGCACGAGTGGGTT
ACATCGAACTGGATCTCAACAGCGGTAAGATCCTTGAGAGTTTTCGCCCCGAAGAACGTTTTCCAATGAT
GAGCACTTTTAAAGTTCTGCTATGTGGCGCGGTATTATCCCGTATTGACGCCGGGCAAGAGCAACTCGGT
CGCCGCATACACTATTCTCAGAATGACTTGGTTGAGTACTCACCAGTCACAGAAAAGCATCTTACGGATG
GCATGACAGTAAGAGAATTATGCAGTGCTGCCATAACCATGAGTGATAACACTGCGGCCAACTTACTTCT
GACAACGATCGGAGGACCGAAGGAGCTAACCGCTTTTTTGCACAACATGGGGGATCATGTAACTCGCCTT
GATCGTTGGGAACCGGAGCTGAATGAAGCCATACCAAACGACGAGCGTGACACCACGATGCCTGTAGCAA
TGGCAACAACGTTGCGCAAACTATTAACTGGCGAACTACTTACTCTAGCTTCCCGGCAACAATTAATAGA
CTGGATGGAGGCGGATAAAGTTGCAGGACCACTTCTGCGCTCGGCCCTTCCGGCTGGCTGGTTTATTGCT
GATAAATCTGGAGCCGGTGAGCGTGGGTCTCGCGGTATCATTGCAGCACTGGGGCCAGATGGTAAGCCCT
CCCGTATCGTAGTTATCTACACGACGGGGAGTCAGGCAACTATGGATGAACGAAATAGACAGATCGCTGA
GATAGGTGCCTCACTGATTAAGCATTGGTAACTGTCAGACCAAGTTTACTCATATATACTTTAGATTGAT
TTAAAACTTCATTTTTAATTTAAAAGGATCTAGGTGAAGATCCTTTTTGATAATCTCATGACCAAAATCC
CTTAACGTGAGTTTTCGTTCCACTGAGCGTCAGACCCCGTAGAAAAGATCAAAGGATCTTCTTGAGATCC
TTTTTTTCTGCGCGTAATCTGCTGCTTGCAAACAAAAAAACCACCGCTACCAGCGGTGGTTTGTTTGCCG
GATCAAGAGCTACCAACTCTTTTTCCGAAGGTAACTGGCTTCAGCAGAGCGCAGATACCAAATACTGTCC
TTCTAGTGTAGCCGTAGTTAGGCCACCACTTCAAGAACTCTGTAGCACCGCCTACATACCTCGCTCTGCT
AATCCTGTTACCAGTGGCTGCTGCCAGTGGCGATAAGTCGTGTCTTACCGGGTTGGACTCAAGACGATAG
TTACCGGATAAGGCGCAGCGGTCGGGCTGAACGGGGGGTTCGTGCACACAGCCCAGCTTGGAGCGAACGA
CCTACACCGAACTGAGATACCTACAGCGTGAGCTATGAGAAAGCGCCACGCTTCCCGAAGGGAGAAAGGC
GGACAGGTATCCGGTAAGCGGCAGGGTCGGAACAGGAGAGCGCACGAGGGAGCTTCCAGGGGGAAACGCC
TGGTATCTTTATAGTCCTGTCGGGTTTCGCCACCTCTGACTTGAGCGTCGATTTTTGTGATGCTCGTCAG
GGGGGCGGAGCCTATGGAAAAACGCCAGCAACGCGGCCTTTTTACGGTTCCTGGCCTTTTGCTGGCCTTT
TGCTCACATGT

Preferably, the recombinant vector of the invention may comprise a nucleic acid sequence which enhances expression of tyrosine hydroxylase (TH) and GTP cyclohydrolase 1 (GCH1). More preferably, the nucleic acid sequence comprises or consists of an optimised intron with pUC origin and RNA-OUT (OIPR) sequence, as described in Lu et al, 2017, “A 5′ Noncoding Exon Containing Engineered Intron Enhances Transgene Expression from Recombinant AAV Vectors in vivo”, Human Gene Therapy, Volume 28, Page 125-134 and WO2013119371.

The OIPR sequence may be referred to herein as SEQ ID No: 26, as follows:

[SEQ ID No: 26]
ATTGGGATCTTCACACAGCAGGTAAGGTTGCGGGCCGGGCCTGGGCCGGGTCCGGGCCGGGTATTGCCCG
CCTAATGAGCGGGCTTTTTTTTCTTACCCCTTCTTCCGCTTCCTCGCTCACTGACTCGCTGCGCTCGGTC
GTTCGGCTGCGGCGAGCGGTATCAGCTCACTCAAAGGCGGTAATACGGTTATCCACAGAATCAGGGGATA
ACGCAGGAAAGAACATGTGAGCAAAAGGCCAGCAAAAGGCCAGGAACCGTAAAAAGGCCGCGTTGCTGGC
GTTTTTCCATAGGCTCCGCCCCCCTGACGAGCATCACAAAAATCGACGCTCAAGTCAGAGGTGGCGAAAC
CCGACAGGACTATAAAGATACCAGGCGTTTCCCCCTGGAAGCTCCCTCGTGCGCTCTCCTGTTCCGACCC
TGCCGCTTACCGGATACCTGTCCGCCTTTCTCCCTTCGGGAAGCGTGGCGCTTTCTCATAGCTCACGCTG
TAGGTATCTCAGTTCGGTGTAGGTCGTTCGCTCCAAGCTGGGCTGTGTGCACGAACCCCCCGTTCAGCCC
GACCGCTGCGCCTTATCCGGTAACTATCGTCTTGAGTCCAACCCGGTAAGACACGACTTATCGCCACTGG
CAGCAGCCACTGGTAACAGGATTAGCAGAGCGAGGTATGTAGGCGGTGCTACAGAGTTCTTGAAGTGGTG
GCCTAACTACGGCTACACTAGAAGAACAGTATTTGGTATCTGCGCTCTGCTGAAGCCAGTTACCTTCGGA
AAAAGAGTTGGTAGCTCTTGATCCGGCAAACAAACCACCGCTGGTAGCGGTGGTTTTTTTGTTTGCAAGC
AGCAGATTACGCGCAGAAAAAAAGGATCTCAAGAAGATCCTTTGATCTTTTCTACGGGGTCTGACGCTCA
GTGGAACGAAAACTCACGTTAAGGGATTTTGGTCATGAGATTATCAAAAAGGATCTTCACCTAGATCCTT
TTAAATTAAAAATGAAGTTTTAAATCAATCTAAAGTATATATGAGTAAACTTGGTCTGACAGTTACCAAT
GCTTAATCAGTGAGGCACCTATCTCAGCGATCTGTCTATTTCGTTCATCCATAGTTGCCTGACTCCTGCA
AACCACGTTGTGGTAGAATTGGTAAAGAGAGTCGTGTAAAATATCGAGTT
CGCACATCTTGTTGTCTGATTATTGATTTTTGGCGAAACCATTTGATCATATGACAAGATGTGTATCTAC
CTTAACTTAATGATTTTGATAAAAATCATTAGGTACCCCGGCCCGCACTGACCCCTGGTGTTGCTTTTTT
TTTTTAGGCCGCAAGCTGAAGCGTGTCC

Preferably, the OIPR sequence comprises a nucleic acid sequence substantially as set out in SEQ ID No: 26, or a fragment or variant thereof.

Preferably, the OIPR sequence is located within the main cassette and is disposed 3′ of the promoter sequence, and 5′ of the coding sequences of tyrosine hydroxylase (TH) and GTP cyclohydrolase 1 (GCH1).

The following sequence, referred to herein as SEQ ID NO: 34, depicts a preferred vector for use according to the first aspect of the invention, the vector comprising, 5′ to 3′, a CMV enhancer, CMV promoter, a sequence encoding truncated human tyrosine hydroxylase (TH) (i.e. excluding the regulatory domain), an F2A linker, furin cleavage site, a sequence encoding human GCH1, a woodchuck hepatitis virus posttranscriptional regulatory element (WPRE) modified to prevent expression of X-protein and SV40pA, in series between two AAV2 inverted terminal repeats (ITRs).

[SEQ ID No: 34]
gcgatcgcggctoccgacatcttggaccattagctccacaggtatcttcttccctctagtggtcataaca
gcagcttcagctacctctcaattcaaaaaacccctcaagacccgtttagaggccccaaggggttatgcta
tcaatcgttgcgttacacacacaaaaaaccaacacacatccatcttcgatggatagcgattttattatct
aactgctgatcgagtgtagccagatctagtaatcaattacggggtcattagttcatagcccatatatgga
gttccgcgttacataacttacggtaaatggcccgcctggctgaccgcccaacgacccccgcccattgacg
tcaataatgacgtatgttcccatagtaacgccaatagggactttccattgacgtcaatgggtggagtatt
tacggtaaactgcccacttggcagtacatcaagtgtatcatatgccaagtacgccccctattgacgtcaa
tgacggtaaatggcccgcctggcattatgcccagtacatgaccttatgggactttcctacttggcagtac
atctacgtattagtcatcgctattaccatgctgatgcggttttggcagtacatcaatgggcgtggatagc
ggtttgactcacggggatttccaagtctccaccccattgacgtcaatgggagtttgttttggcaccaaaa
tcaacgggactttccaaaatgtcgtaacaactccgccccattgacgcaaatgggcggtaggcgtgtacgg
tgggaggtctatataagcagagctggtttagtgaaccgtcagatcagatctttgtcgatcctaccatcca
ctcgacacacccgccagcggccgctgccaagcttccgagctctcgaattcaaaggaggtacccaccatgg
ccaccatgagccccgcggggcccaaggtcccctggttcccaagaaaagtgtcagagctggacaagtgtca
tcacctggtcaccaagttcgaccctgacctggacttggaccacccgggcttctcggaccaggtgtaccgc
cagcgcaggaagctgattgctgagatcgccttccagtacaggcacggcgacccgattccccgtgtggagt
acaccgccgaggagattgccacctggaaggaggtctacaccacgctgaagggcctctacgccacgcacgc
ctgcggggagcacctggaggcctttgctttgctggagcgcttcagcggctaccgggaagacaatatcccc
cagctggaggacgtctcccgcttcctgaaggagcgcacgggcttccagctgcggcctgtggccggcctgc
tgtccgcccgggacttcctggccagcctggccttccgcgtgttccagtgcacccagtatatccgccacgc
gtcctcgcccatgcactcccctgagccggactgctgccacgagctgctggggcacgtgcccatgctggcc
gaccgcaccttcgcgcagttctcgcaggacattggcctggcgtccctgggggcctcggatgaggaaattg
agaagctgtccacgctgtactggttcacggtggagttcgggctgtgtaagcagaacggggaggtgaaggc
ctatggtgccgggctgctgtcctcctacggggagctcctgcactgcctgtctgaggagcctgagattcgg
gccttcgaccctgaggctgcggccgtgcagccctaccaagaccagacgtaccagtcagtctacttcgtgt
ctgagagcttcagtgacgccaaggacaagctcaggagctatgcctcacgcatccagcgccccttctccgt
gaagttcgacccgtacacgctggccatcgacgtgctggacagcccccaggccgtgcggcgctccctggag
ggtgtccaggatgagctggacacccttgcccatgcgctgagtgccattggccgcgcgaaacgcgcgccgg
tgaaacagaccctgaactttgatctgctgaaactggcgggcgatgtggaaagcaacccgggcccgatgga
gaagggccctgtgcgggcaccggcggagaagccgcggggcgccaggtgcagcaatgggttccccgagcgg
gatccgccgcggcccgggcccagcaggccggcggagaagcccccgcggcccgaggccaagagcgcgcagc
ccgcggacggctggaagggcgagcggccccgcagcgaggaggataacgagctgaacctccctaacctggc
agccgcctactcgtccatcctgagctcgctgggcgagaacccccagcggcaagggctgctcaagacgccc
tggagggcggcctcggccatgcagttcttcaccaagggctaccaggagaccatctcagatgtcctaaacg
atgctatatttgatgaagatcatgatgagatggtgattgtgaaggacatagacatgttttccatgtgtga
gcatcacttggttccatttgttggaaaggtccatattggttatcttcctaacaagcaagtccttggcctc
agcaaacttgcgaggattgtagaaatctatagtagaagactacaagttcaggagcgccttacaaaacaaa
ttgctgtagcaatcacggaagccttgcggcctgctggagtcggggtagtggttgaagcaacacacatgtg
tatggtaatgcgaggtgtacagaaaatgaacagcaaaactgtgaccagcacaatgttgggtgtgttccgg
gaggatccaaagactcgggaagagttcctgactctcattaggagctgagccacctaatcaacctctggat
tacaaaatttgtgaaagattgactggtattcttaactatgttgctccttttacgctatgtggatacgctg
ctttaatgcctttgtatcatgctattgcttcccgtatggctttcattttctcctccttgtataaatcctg
gttgctgtctctttatgaggagttgtggcccgttgtcaggcaacgtggcgtggtgtgcactgtgtttgct
gacgcaacccccactggttggggcattgccaccacctgtcagctcctttccgggactttcgctttccccc
tccctattgccacggcggaactcatcgccgcctgccttgcccgctgctggacaggggctcggctgttggg
cactgacaattccgtggtgttgtcggggaaatcatcgtcctttccctggctgactgatacaatcgatttc
tggatccgcaggcctctgctagcttgactgactgagatacagcgtaccttcagctcacagacatgataag
atacattgatgagtttggacaaaccacaactagaatgcagtgaaaaaaatgctttatttgtgaaatttgt
gatgctattgctttatttgtaaccattataagctgcaataaacaagttaacaacaacaattgcattcatt
ttatgtttcaggttcagggggaggtgtgggaggttttttaaagcaagtaaaacctctacaaatgtggtag
tcgtcagctatcctgcaggcgatctctcgatttcgatcaagacattcctttaatggtcttttctggacac
cactaggggtcagaagtagttcatcaaactttcttccctccctaatctcattggttaccttgggctatcg
aaacttaattaaccagtcaagtcagctacttggcgagatcgacttgtctgggtttcgactacgctcagaa
ttgcgtcagtcaagttcgatctggtccttgctattgcacccgttctccgattacgagtttcatttaaatc
atgtgagcaaaaggccagcaaaaggccaggaaccgtaaaaaggccgcgttgctggcgtttttccataggc
tccgcccccctgacgagcatcacaaaaatcgacgctcaagtcagaggtggcgaaacccgacaggactata
aagataccaggcgtttccccctggaagctccctcgtgcgctctcctgttccgaccctgccgcttaccgga
tacctgtccgcctttctcccttcgggaagcgtggcgctttctcatagctcacgctgtaggtatctcagtt
cggtgtaggtcgttcgctccaagctgggctgtgtgcacgaaccccccgttcagcccgaccgctgcgcctt
atccggtaactatcgtcttgagtccaacccggtaagacacgacttatcgccactggcagcagccactggt
aacaggattagcagagcgaggtatgtaggcggtgctacagagttcttgaagtggtggcctaactacggct
acactagaagaacagtatttggtatctgcgctctgctgaagccagttaccttcggaaaaagagttggtag
ctcttgatccggcaaacaaaccaccgctggtagcggtggtttttttgtttgcaagcagcagattacgcgc
agaaaaaaaggatctcaagaagatcctttgatcttttctacggggtctgacgctcagtggaacgaaaact
cacgttaagggattttggtcatgagattatcaaaaaggatcttcacctagatccttttaaattaaaaatg
aagttttaaatcaatctaaagtatatatgagtaaacttggtctgacagttaccaatgcttaatcagtgag
gcacctatctcagcgatctgtctatttcgttcatccatagttgcatttaaatttccgaactctccaaggc
cctcgtcggaaaatcttcaaacctttcgtccgatccatcttgcaggctacctctcgaacgaactatcgca
agtctcttggccggccttgcgccttggctattgcttggcagcgcctatcgccaggtattactccaatccc
gaatatccgagatcgggatcacccgagagaagttcaacctacatcctcaatcccgatctatccgagatcc
gaggaatatcgaaatcggggcgcgcctggtgtaccgagaacgatcctctcagtgcgagtctcgacgatcc
atatcgttgcttggcagtcagccagtcggaatccagcttgggacccaggaagtccaatcgtcagatattg
tactcaagcctggtcacggcagcgtaccgatctgtttaaacctagatattgatagtctgatcggtcaacg
tataatcgagtcctagcttttgcaaacatctatcaagagacaggatcagcaggaggctttcgcatgattg
aacaagatggattgcacgcaggttctccggcggcttgggtggagaggctattcggctatgactgggcaca
acagacaatcggctgctctgatgccgccgtgttccggctgtcagcgcaggggcgtccggttctttttgtc
aagaccgacctgtccggtgccctgaatgaactgcaagacgaggcagcgcggctatcgtggctggcgacga
cgggcgttccttgcgcggctgtgctcgacgttgtcactgaagcgggaagggactggctgctattgggcga
agtgccggggcaggatctcctgtcatctcaccttgctcctgccgagaaagtatccatcatggctgatgca
atgcggcggctgcatacgcttgatccggctacctgcccattcgaccaccaagcgaaacatcgcatcgagc
gagcacgtactcggatggaagccggtcttgtcgatcaggatgatctggacgaagagcatcaggggctcgc
gccagccgaactgttcgccaggctcaaggcgtctatgcccgacggcgaggatctcgtcgtgacccacggc
gatgcctgcttgccgaatatcatggtggaaaatggccgcttttctggattcatcgactgtggccgtctgg
gtgtggcggaccgctatcaggacatagcgttggctacccgtgatattgctgaagagcttggcggcgaatg
ggctgaccgcttccttgtgctttacggtatcgccgcgcccgattcgcagcgcatcgccttctatcgcctt
cttgacgagttcttctgaccgattctaggtgcattggcgcagaaaaaaatgcctgatgcgacgctgcgcg
tcttatactcccacatatgccagattcagcaacggatacggcttccccaacttgcccacttccatacgtg
tcctccttaccagaaatttatccttaaggtcgtttaaactcgactctggctctatcgaatctccgtcgtt
tcgagcttacgcgaacagccgtggcgctcatttgctcgtcgggcatcgaatctcgtcagctatcgtcagc
ttacctttttggca

Preferably, the vector comprises a nucleic acid sequence substantially as set out in SEQ ID No: 34, or a fragment or variant thereof.

The gene therapy vectors may be produced by any technique known in the art. For instance, the rAAV vectors may be produced using classic triple transfection methodology. Methods for the production of adeno-associated virus vectors are disclosed in Matsushita et al. (Matsushita et al., Adeno-associated virus vectors can be efficiently produced without helper virus. Gene Therapy (1998) 5, 938-945)

In one embodiment the genome sequence described herein, i.e. the promoter-TH-linker-GCH1 sequence, or promoter-TH-linker-GCH1-linker-PTPS sequence, may be administered by injection directly as naked DNA without a viral vector. The naked DNA may be administered as a plasmid, minicircle, nanoplasmid or mini-intron plasmid (MIP). The naked DNA may be delivered as a plasmid administered in any suitable non-viral carrier that would be known to those skilled in the art.

Preferably, the non-viral carrier is selected from the groups consisting of: poly(2-ethyl-2-oxazoline)-PLA-g-PEI amphiphilic triblock micelles, a Poly(β-amino ester)-based biodegradable nanoparticle, a Pluronic® block-copolymer such as Pluronic F27, Pluronic F68 or Pluronic F85 a mixture of Pluronics such as SP1017, and a carrier such as BrainFectIn® (OZ Biosciences, Marseille, France).

It will be appreciated that the amount of the genetic construct or the recombinant vector that is required is determined by its biological activity and bioavailability, which in turn depends on the mode of administration, the physiochemical properties of the genetic construct or the recombinant vector and whether it is being used as a monotherapy or in a combined therapy. Optimal dosages to be administered may be determined by those skilled in the art, and will vary with the particular genetic construct or the recombinant vector in use, the strength of the pharmaceutical composition, the mode of administration, and the advancement of the neurodegenerative disorder. Additional factors depending on the particular subject being treated will result in a need to adjust dosages, including subject age, weight, gender, diet, and time of administration.

The dose delivered may be 300 μl to 20,000 μl, 300 μl to 10,000 μl, 300 μl to 5,000 μl, 300 μl to 4500 μl, 400 μl to 4000 μl, 500 μl to 3500 μl, 600 μl to 3000 μl, 700 μl to 2500 μl, 750 μl to 2000 μl, 800 μl to 1500 μl, 850 μl to 1000 μl, or roughly 900 μl.

The titre of the dose may be 1E8 to 5E14, 1E9 to 1E14, 1E10 to 5E13, 1E11 to 1E13, 1E12 to 8E12, 4E12 to 6E12, or roughly 5E12 genome copies per ml (GC/ml).

The genetic construct or the recombinant vector may be administered before, during or after onset of the disorder. Doses may be given as a single administration, or multiple doses may be given over the course of the treatment. A dose may be administered to a patient, and the patient may be monitored in order to assess the necessity for a second or further doses. Repeat use delivery of the same genome may be facilitated by the switching the AAV capsid serotype to reduce the probability of interference by an antibody or cell mediated immune response induced by the previous treatment.

In some embodiments, the therapeutic methods may include, prior to gene therapy treatment, a test infusion of L-DOPA. The test infusion may be used to demonstrate that a subject is responsive to L-DOPA, and so may allow the selection of subjects most likely to benefit from gene therapy treatment. The L-DOPA test infusion may be by any means capable of creating a steady blood level over hours or days. Examples of suitable infusion methods include by nasogastric tube, i.v. infusion, infusion via a pump, by the use of DuoDOPA, or any other suitable means.

It will be appreciated that the genetic construct according to the first aspect, or the recombinant vector according to the second aspect may be used in a medicament, which may be used as a monotherapy (i.e. use of the genetic construct according to the first aspect or the vector according to the second aspect of the invention), for treating, ameliorating, or preventing any disorder as disclosed herein. Alternatively, the genetic construct or the recombinant vector according to the invention may be used as an adjunct to, or in combination with, known therapies for treating, ameliorating, or preventing any disorder as disclosed herein. In some cases, the genetic construct may be used as an adjunct to, in combination with, or alongside a treatment designed to improve the gene therapy. For instance, the genetic construct may be used in combination with an immunosuppressive treatment, in order to reduce, prevent, or control an immune response induced by the gene therapy itself. For example, the immunosuppressive treatment may prevent, reduce, or control an immune response directed to a capsid of a gene therapy vector, a genome comprised within a gene therapy vector, or a product produced by a gene therapy vector during therapy. The immunosuppressive regime may include a general immunosuppressant, such as steroid. The immunosuppressive regime may include more targeted immunosuppression designed to reduce specific immune responses, such as immunotherapy to specific antigens found within, or produced by, a gene therapy construct.

The genetic construct according or the recombinant vector according to the invention may be combined in compositions having a number of different forms depending, in particular, on the manner in which the composition is to be used. Thus, for example, the composition may be in the form of a powder, liquid, micellar solution, liposome suspension or any other suitable form that may be administered to a person or animal in need of treatment. It will be appreciated that the vehicle of medicaments according to the invention should be one which is well-tolerated by the subject to whom it is given. Preferably, the composition is in the form of an injectable liquid.

Known procedures, such as those conventionally employed by the pharmaceutical industry (e.g. in vivo experimentation, clinical trials, etc.), may be used to form specific formulations of the genetic construct or the recombinant vector according to the invention and precise therapeutic regimes.

According to a third aspect, there is provided a pharmaceutical composition comprising the genetic construct according to the first aspect, or the recombinant vector according to the second aspect, and a pharmaceutically acceptable vehicle, for use in treating, preventing, or ameliorating a neurodegenerative disorder, wherein the pharmaceutical composition is delivered to the cerebrospinal fluid (CSF) of a subject.

Preferably, the delivery and neurodegenerative disorder is as defined in the first aspect. Preferably, however, the composition is an injectable composition.

A “subject” may be a vertebrate, mammal, or domestic animal. Hence, compositions and medicaments according to the invention may be used to treat any mammal, for example livestock (e.g. a horse), pets, or may be used in other veterinary applications. Most preferably, however, the subject is a human being.

A “therapeutically effective amount” of the genetic construct, the recombinant vector or the pharmaceutical composition is any amount which, when administered to a subject, is the amount of the aforementioned that is needed to treat the disorder.

For example, the therapeutically effective amount of the genetic construct, the recombinant vector or the pharmaceutical composition used may be from about 0.01 mg to about 800 mg, and preferably from about 0.01 mg to about 500 mg. It is preferred that the amount of the genetic construct, the recombinant vector or the pharmaceutical composition is an amount from about 0.1 mg to about 250 mg, and most preferably from about 0.1 mg to about 20 mg.

A “pharmaceutically acceptable vehicle” as referred to herein, is any known compound or combination of known compounds that are known to those skilled in the art to be useful in formulating pharmaceutical compositions.

In a preferred embodiment, the pharmaceutically acceptable vehicle may be such as to allow injection of the composition directly into a subject. For instance, the vehicle may be suitable for allowing the injection of the composition into the CSF.

In one embodiment, the pharmaceutically acceptable vehicle may be a solid, and the composition may be in the form of a powder, or suspension. A solid pharmaceutically acceptable vehicle may include one or more substances which may also act as, lubricants, solubilisers, suspending agents, dyes, fillers, glidants, compression aids, inert binders, preservatives, dyes, coatings, or solid-disintegrating agents. The vehicle may also be an encapsulating material. In powders, the vehicle is a finely divided solid that is in admixture with the finely divided active agents according to the invention. In another embodiment, the pharmaceutical vehicle may be a gel or the like.

However, the pharmaceutical vehicle may be a suspension or a liquid, and the pharmaceutical composition is in the form of a suspension or a solution. Liquid pharmaceutical compositions, which are sterile solutions or suspensions, can be utilized by, for example, intramuscular, intrathecal, epidural, intraperitoneal, intravenous and particularly subcutaneous injection. The genetic construct or the recombinant vector may be prepared as a sterile solid composition that may be dissolved or suspended at the time of administration using sterile water, saline, Dulbecco's Phosphate Buffered Saline (dPBS) with MgCl2 and CaCl2), or other appropriate sterile injectable medium.

It will be appreciated that the invention extends to any nucleic acid or peptide or variant, derivative or analogue thereof, which comprises substantially the amino acid or nucleic acid sequences of any of the sequences referred to herein, including variants or fragments thereof. The terms “substantially the amino acid/nucleotide/peptide sequence”, “variant” and “fragment”, can be a sequence that has at least 40% sequence identity with the amino acid/nucleotide/peptide sequences of any one of the sequences referred to herein, for example 40% identity with the sequence identified as SEQ ID No:1-44, and so on.

Amino acid/polynucleotide/polypeptide sequences with a sequence identity which is greater than 65%, more preferably greater than 70%, even more preferably greater than 75%, and still more preferably greater than 80% sequence identity to any of the sequences referred to are also envisaged. Preferably, the amino acid/polynucleotide/polypeptide sequence has at least 85% identity with any of the sequences referred to, more preferably at least 90% identity, even more preferably at least 92% identity, even more preferably at least 95% identity, even more preferably at least 97% identity, even more preferably at least 98% identity and, most preferably at least 99% identity with any of the sequences referred to herein.

The skilled technician will appreciate how to calculate the percentage identity between two amino acid/polynucleotide/polypeptide sequences. In order to calculate the percentage identity between two amino acid/polynucleotide/polypeptide sequences, an alignment of the two sequences must first be prepared, followed by calculation of the sequence identity value. The percentage identity for two sequences may take different values depending on: (i) the method used to align the sequences, for example, ClustalW, BLAST, FASTA, Smith-Waterman (implemented in different programs), or structural alignment from 3D comparison; and (ii) the parameters used by the alignment method, for example, local vs global alignment, the pair-score matrix used (e.g. BLOSUM62, PAM250, Gonnet etc.), and gap-penalty, e.g. functional form and constants.

Having made the alignment, there are many different ways of calculating percentage identity between the two sequences. For example, one may divide the number of identities by: (i) the length of shortest sequence; (ii) the length of alignment; (iii) the mean length of sequence; (iv) the number of non-gap positions; or (v) the number of equivalenced positions excluding overhangs. Furthermore, it will be appreciated that percentage identity is also strongly length dependent. Therefore, the shorter a pair of sequences is, the higher the sequence identity one may expect to occur by chance.

Hence, it will be appreciated that the accurate alignment of protein or DNA sequences is a complex process. The popular multiple alignment program ClustalW (Thompson et al., 1994, Nucleic Acids Research, 22, 4673-4680; Thompson et al., 1997, Nucleic Acids Research, 24, 4876-4882) is a preferred way for generating multiple alignments of proteins or DNA in accordance with the invention. Suitable parameters for ClustalW may be as follows: For DNA alignments: Gap Open Penalty=15.0, Gap Extension Penalty=6.66, and Matrix=Identity. For protein alignments: Gap Open Penalty=10.0, Gap Extension Penalty=0.2, and Matrix=Gonnet. For DNA and Protein alignments: ENDGAP=−1, and GAPDIST=4. Those skilled in the art will be aware that it may be necessary to vary these and other parameters for optimal sequence alignment.

Preferably, calculation of percentage identities between two amino acid/polynucleotide/polypeptide sequences may then be calculated from such an alignment as (N/T)*100, where N is the number of positions at which the sequences share an identical residue, and T is the total number of positions compared including gaps and either including or excluding overhangs. Preferably, overhangs are included in the calculation. Hence, a most preferred method for calculating percentage identity between two sequences comprises (i) preparing a sequence alignment using the ClustalW program using a suitable set of parameters, for example, as set out above; and (ii) inserting the values of N and T into the following formula: Sequence Identity=(N/T)*100.

Alternative methods for identifying similar sequences will be known to those skilled in the art. For example, a substantially similar nucleotide sequence will be encoded by a sequence which hybridizes to DNA sequences or their complements under stringent conditions. By stringent conditions, we mean the nucleotide hybridises to filter-bound DNA or RNA in 3× sodium chloride/sodium citrate (SSC) at approximately 45° C. followed by at least one wash in 0.2×SSC/0.1% SDS at approximately 20-65° C. Alternatively, a substantially similar polypeptide may differ by at least 1, but less than 5, 10, 20, 50 or 100 amino acids from the sequences shown in, for example, in the amino acid sequence that are included within SEQ ID Nos: 1-44.

Due to the degeneracy of the genetic code, it is clear that any nucleic acid sequence described herein could be varied or changed without substantially affecting the sequence of the protein encoded thereby, to provide a functional variant thereof. Suitable nucleotide variants are those having a sequence altered by the substitution of different codons that encode the same amino acid within the sequence, thus producing a silent change. Other suitable variants are those having homologous nucleotide sequences but comprising all, or portions of, sequence, which are altered by the substitution of different codons that encode an amino acid with a side chain of similar biophysical properties to the amino acid it substitutes, to produce a conservative change. For example, small non-polar, hydrophobic amino acids include glycine, alanine, leucine, isoleucine, valine, proline, and methionine. Large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine. The negatively charged (acidic) amino acids include aspartic acid and glutamic acid. It will therefore be appreciated which amino acids may be replaced with an amino acid having similar biophysical properties, and the skilled technician will know the nucleotide sequences encoding these amino acids.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying Figures, in which:

FIG. 1 is a plasmid map of a first embodiment of the construct of the invention, showing the features of SEQ ID NO: 13;

FIG. 2 is a plasmid map of a second embodiment of the construct of the invention, showing the features of SEQ ID NO: 14;

FIG. 3 is a plasmid map of a third embodiment of the construct of the invention, showing the features of SEQ ID NO: 15;

FIG. 4 is a plasmid map of a fourth embodiment of the construct of the invention, showing the features of SEQ ID NO: 16;

FIG. 5 is a plasmid map of a fifth embodiment of the construct of the invention, showing the features of SEQ ID NO: 17;

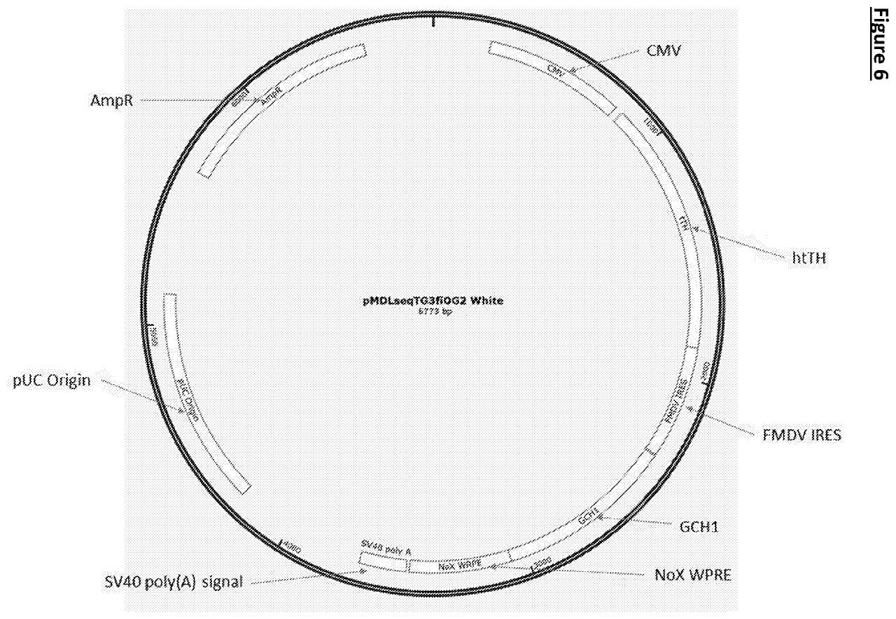

FIG. 6 is a plasmid map of a sixth embodiment of the construct of the invention, showing the features of SEQ ID NO: 18;

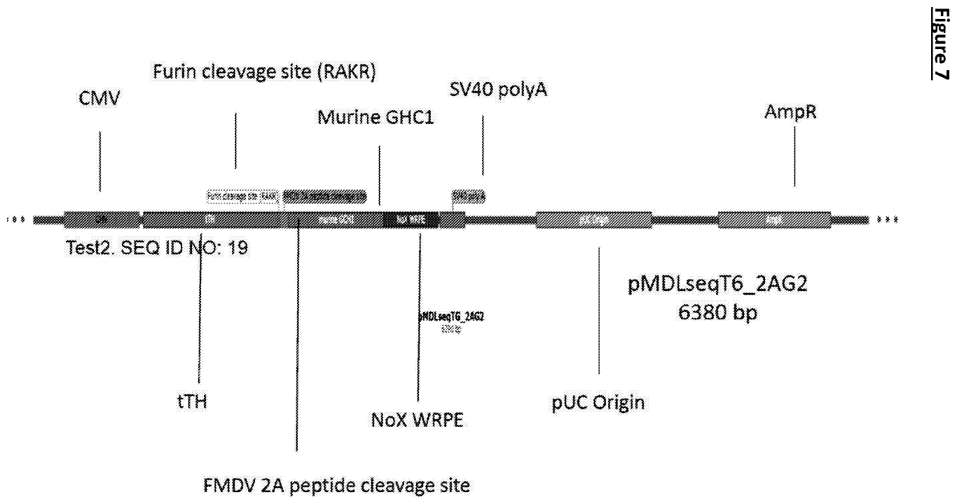

FIG. 7 is a plasmid map of a seventh embodiment of the construct of the invention, showing the features of SEQ ID NO: 19;

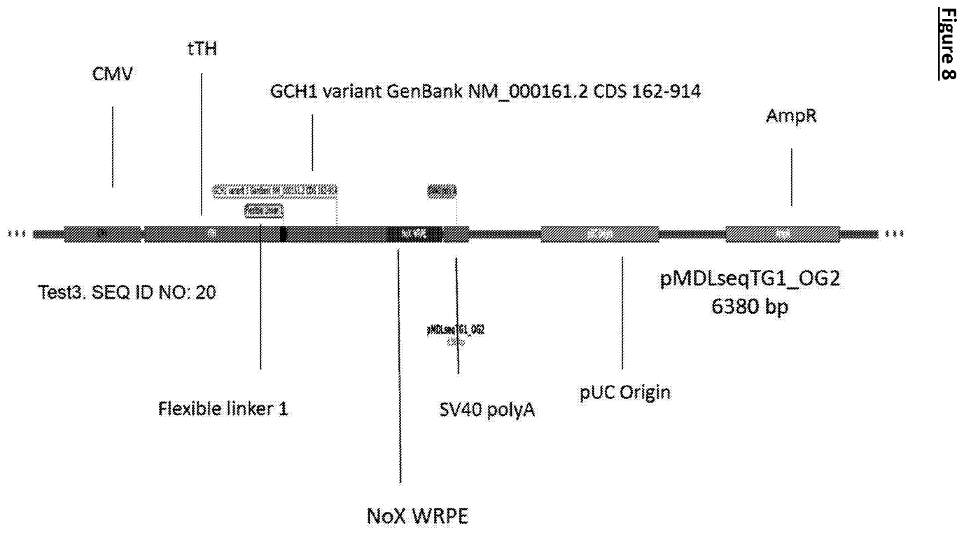

FIG. 8 is a plasmid map of an eighth embodiment of the construct of the invention, showing the features of SEQ ID NO: 20;

FIG. 9 shows that rats treated by intrathecal injection of one embodiment of the construct of the invention display increased L-DOPA levels in the CSF after 14 days. Pre-AAV baseline DOPA is the first control referring to the DOPA concentration at the initial time-point of injection (pre-AAV), and the second control is the level of DOPA in a subject not exposed to the construct at 14 days (no AAV);

FIG. 10 shows that rats treated by intrathecal injection of one embodiment of the construct of the invention display increased dopamine levels in the CSF. The control refers to the dopamine concentration at the initial time-point of injection (pre-AAV); and

FIG. 11 shows that rats treated by intrathecal injection of one embodiment of the construct of the invention display reduced intracellular dopamine levels in the striatum. The control refers to the dopamine concentration 14 days after AAV administration (14 day no AAV control).

FIG. 12 shows that intrathecal injection into either the lateral intracerebral ventricle or the cisterna magna produced a similar reduction in striatal intracellular dopamine levels.

### EXAMPLES

### Background

Previous studies for gene therapy for Parkinson's disease have assumed that for successful treatment, vectors for gene therapy would need to be transferred directly into the patient's striatum, where the vector carries genes necessary for the production of dopamine or L-DOPA by brain cells that would ordinarily be non-dopamine producing. The aim of such treatment is the local generation of dopamine within the affected areas of the brains of Parkinson's patients. Several methods of gene therapy have been disclosed. However, while the technique has shown some promise, and the previous methods provide a proof of the principle, previous vectors have not been optimal, and are associated with brain surgery risks. In particular, there has been a need for vectors and delivery means that leads to optimal production of dopamine (either directly or indirectly via L-DOPA) in the brains of Parkinson's patients, and which can be manufactured at suitable levels and with suitable cost effectiveness to be a viable treatment option, and which do not suffer the risks and complexities associated with direct injection into the striatum, putamen, caudate or substantia nigra.

The inventor hypothesised that by injecting the AAV into the intrathecal space—i.e. into the cerebrospinal fluid—it is possible to raise the CSF and brain extracellular fluid levels of L-DOPA and use this as a route of impacting the dopamine level in the striatum of patients with PD. Although this would expose the entire brain to increased levels of L-DOPA, this should be similar to what happens when patients are treated with classical oral L-DOPA. The latter has been the gold standard for the treatment of PD for more than 40 years and the “whole brain” impact of L-DOPA is usually well-tolerated in the majority of patients.

Based on the inventor's hypothesis, he performed a study in rats using two routes to administer constructs of the invention into the CSF, either a single simple injection into the intracerebral ventricle system or a single simple injection into the cisterna magna.

Materials and Methods

Construct/Vector

A bicistronic AAV (serotype 9) was used prepared by triple transfection. The vector genome included a CMV enhancer, CMV promoter, cDNA for human tyrosine hydroxylase (excluding the regulatory domain), an F2A linker, furin cleavage site, cDNA for human GCH1, a woodchuck hepatitis virus posttranscriptional regulatory element (WPRE) modified to prevent expression of X-protein and SV40pA, in series between two AAV2 inverted terminal repeats (ITRs).

OHDA Lesion of the MFB

Unilateral lesions of the nigrastriatal pathway were conducted by intracerebral administration of 6-hydroxydopamine (6-OHDA). 6-OHDA was formulated at 5 mg/ml solution in 0.03% ascorbic acid in sterile 0.90% NaCl. Three μL of 6-OHDA was injected into the medial forebrain bundle at the following stereotaxic coordinates from bregma: Anteroposterior (A/P) −4.0 mm; mediolateral (M/L) −1.3 mm; ventrodorsal (V/D) −8.0 mm with reference to top of skull.

ICV Injection of TA and CSF Collection

Two weeks after 6-OHDA lesion, animals were randomized into the treatment groups. Animals from Group 2 were anesthetized with isoflurane and placed in the stereotaxic frame with a nose bar set a +5 mm. A 2 cm sagittal incision was made to locate bregma. A hole was drilled using the following coordinates: AP: −0.4; L: +2.0. CSF (˜50 μl) was drawn from the ventricle (using a Hamilton syringe lowered at −4.5 mm).

For CM collection, rats were anesthetized with isoflurane and positioned in the stereotaxic frame. The rat head was flexed downward at approximately 45 degrees, a depressible surface with the appearance of a rhomb between occipital protuberances and the spine of the atlas was visible. The 23 G needle was punctured into the cisterna magna for CSF collection without making any incision at this region.

The AAV9 vector was slowly infused into the ventricle (10 μl/min) using the same coordinates and the same hole, the volume of injection: 50 μl (TBD). The needle was be left in place for 3 min and then withdrawn. The incision was closed with wound clips. After CM CSF collection, the needle was left in place and then connected to a syringe containing the TA. TA was slowly infused into the CM (10 μl/min) the volume of injection: ˜50 μl (TBD). The needle was left in place for 3 min and then withdrawn. Control animals did not have vector injected.

Terminal CSF Collection and Striatum Dissection

On day 28 days after the 6-OHDA lesions, animals were anesthetized with isoflurane and CSF was collected from the CM, transferred into a clean tube and flash frozen. After CSF collection, animals were sacrificed and brains extracted. Left striatum was dissected, weighed in the tube and flash frozen CSF samples were stored at −80° C. until shipment to client-designated laboratory.

Table 1 shows a summary of the steps that were performed to measure CSF levels after lesion of the basal forebrain and subsequent injection of the bicistronic vector.

| Day | Event |
|---|---|
| Day 1 | Surgery/unilateral lesion of the medial forebrain bundle |
| Day 14 | Surgery: CSF collection and TA injection into the lateral |
| | ventricle or CM |
| Day 28 | Takedown: CSF collection, striatum dissection |

Results and Discussion

FIGS. 1 to 8 show embodiments of the gene therapy vectors used in accordance with the invention described herein. In particular, the vector shown in FIG. 3 was used in the following examples.

### Example 1—DOPA Levels are Elevated in the CSF

The gene therapy vectors described herein are designed to transfect cells of the ependyma and the adjacent tissue in the vicinity of the CSF. The vectors transduce production of tyrosine hydroxylase and GCH1 (the latter is rate-limiting in the production of BH4, which is a cofactor essential for TH activity). FIG. 9 shows that DOPA (=L-DOPA) levels in the CSF display a very highly significant increase in vector-treated animals compared with either pre-treatment or non-treated (no vector) controls.

### Example 2—Dopamine Levels are Elevated in the CSF

It is known that there is residual AADC activity in the Parkinsonian brain, and the fact that oral L-DOPA is active depends on this. While there are a number of views on where this AADC resides (e.g. surviving dopaminergic neurons, interneurons, serotonegic neurons, or a combination of these), the inventor has observed the increased CSF L-DOPA to result in an increase in CSF dopamine concentrations due to this decarboxylation. Indeed, FIG. 10 shows that Dopamine levels in the CSF display a very highly significant increase in vector-treated animals compared with the pre-treatment control.

### Example 3—Striatum Intracellular Dopamine Levels are Reduced

The DOPA and Dopamine produced in this way in the CSF, ependyma and adjacent tissue will be distributed more broadly into the brain via blood or in extracellular fluid pulsating in the perivascular space, and this will enable them to reach the striatum to impart their therapeutic effects. The striatum can be viewed as two compartments (the intracellular compartment and the extracellular fluid compartment), and it will be appreciated that what happens in the extracellular compartment influences what happens intracellularly. In the present invention, dopaminergic cells can detect the amount of dopamine in the extracellular fluid within the striatum. If the extracellular level of dopamine is high, the striatal cells react by reducing their production and subsequent secretion of dopamine.

Assaying the intracellular Dopamine levels in the striatum therefore provides an indicator of whether the increase in L-DOPA production in the ependyma and tissue adjacent to the CSF is:
(a) distributed to non-adjacent tissue; and
(b) sufficient to stimulate dopamine receptors at these non-adjacent sites and therefore to be of therapeutic potential.

FIG. 11 shows that the intracellular striatum Dopamine levels display a very highly significant decrease in vector treated animals compared with the no AAV control. As the intracellular levels of Dopamine are reduced in the vector-treated animals, this is consistent with a subsequent increase in extracellular Dopamine levels. Given that Figure C shows the concentration of intracellular striatal Dopamine concentrations, and the understanding that the therapeutic objective with this invention is to raise L-DOPA levels in the extracellular fluid surrounding the basal ganglia (including the striatum), these data clearly support the view that the vector is achieving its desired effect in that the increases in DOPA and Dopamine are principally in the extracellular fluid compartment of the striatum. The increase in DOPA and Dopamine in the extracellular compartment will result in feedback inhibition of Dopamine production within the surviving dopaminergic cells of the lesioned striatum.

FIG. 12 shows that intrathecal injection into either the lateral intracerebral ventricle or the cisterna magna produced a similar reduction in striatal intracellular dopamine levels.

### SUMMARY

In summary, the use of the constructs described herein displays the following advantages over current methods in the art:

i) the invention is a simple and practical method of treating Parkinson's which addresses the limitations of previously employed methods. The inventor has demonstrated that a gene therapy construct administered in non-targeted manner into the CSF can result in an increase is substrate (DOPA) sufficient to enable local conversion of the neurotransmitter L-DOPA within the therapeutic target (the striatum) and has demonstrated that the resulting extracellular levels of dopamine are sufficient to stimulate and expected result on local dopamine receptors. (ii) provision of constant level of L-DOPA substrate to the CNS. This may replace or reduce the need for oral L-DOPA therapy. By providing a constant level of L-DOPA production, the peaks and troughs associated with oral therapy will be avoided or reduced. This in turn will prevent, or reduce the risk of, or treat long-term complications of L-DOPA therapy that are related to the variable blood levels associated with oral L-DOPA therapy (including dyskinesia, on/off fluctuations and “freezing”);

iii) no need for the requirement of complex, lengthy surgery to infuse gene therapy directly to the striatum. Current gene therapy approaches seeking to increase L-DOPA or dopamine production within the central nervous system infuse vector directly into the striatum. This may require use of multiple needle tracts though brain tissue of both hemispheres in order to ensure adequate distribution of vector over the target tissue. Infusion of vector into brain tissue must be slow to achieve maximum distribution and avoid injury. The resulting procedure must be implemented by a full neurosurgical team in a neurosurgical suite and may take up to 10 hours (usually 4-6 hours). The procedure carries the risk of death or incapacity due to cerebral haemorrhage. In contrast direct injection of vector into the cerebrospinal fluid can be achieved more quickly and simply and at lower risk;

iv) marked reduction in cost of goods versus gene therapy transducing constant peripheral production of L-DOPA (for example from liver and/or muscle). By enabling local production of L-DOPA within the CNS, the invention avoids inefficiency due to peripheral distribution, excretion and metabolism of L-DOPA before it reaches the CNS and reduces the challenge of transfer of L-DOPA across the blood brain barrier. The invention therefore requires a lower dose of vector with a lower cost of goods. The invention avoids the need for many intramuscular injections or complex infusion regimens necessary to adequately transduce liver or muscle and may be less immunogenic;

v) the use results in the production of L-DOPA but does not transduce expression of AADC. Thus, while increasing the level of the dopamine substrate available throughout the CNS (as happens with current standard therapy with oral or enteral administration of L-DOPA) production of dopamine is only increased in areas of brain with significant intrinsic AADC activity. This reduces the risk of off-target dopamine induced toxicity; and

vi) by providing constant levels of DOPA and dopamine in the striatal extracellular fluid the invention achieves the same pharmacological objective as currently achieved by continuous infusion of L-DOPA/carbidopa gel without the need for continuous infusion into the jejunum. The invention will enable the superior efficacy achieved by continuous infusion of L-DOPA/carbidopa gel (Duodopa) but without the lifelong burden of PEG tube and the associated risks of blockage, displacement and infection.

1-26. (canceled)

27. A method of treating, preventing, or ameliorating a neurodegenerative disorder in a subject, wherein the method comprises administering, to a subject in need of such treatment, a genetic construct comprising a promoter operably linked to a first coding sequence, which encodes tyrosine hydroxylase (TH), and a second coding sequence, which encodes GTP cyclohydrolase 1 (GCH1), wherein the second coding sequence is 3′ to the first coding sequence, and the first and second coding sequences are part of a single operon, wherein the genetic construct does not encode aromatic amino acid decarboxylase (AADC), and wherein the construct is delivered to the cerebrospinal fluid (CSF) of the subject.

28. The method according to claim 27, wherein the construct is delivered to the CSF by injection.

29. The method according to claim 27, wherein the genetic construct is delivered to the CSF via one or more of a group selected from: the intracerebral ventricle system; the cisterna magna; and between lumbar vertebrae L3/L4, L4/L5 or L5/S1.

30. The method according to claim 27, wherein the genetic construct is delivered to the CSF via the intracerebral ventricle system.

31. The method according to claim 27, wherein the genetic construct is delivered to the CSF via the cisterna magna.

32. The method according to claim 27, wherein the genetic construct is delivered to the CSF via between lumbar vertebrae L3/L4, L4/L5 or L5/S1.

33. The method according to claim 27, wherein the CSF DOPA level is increased sufficiently to trigger feedback inhibition of dopamine production by surviving dopaminergic cells within the striatum.

34. The method according to claim 27, wherein the CSF DOPA level is increased to between 5 pmol/ml and 20 pmol/ml, between 7 pmol/ml and 15 pmol/ml, or between 8 pmol/ml and 12 pmol/ml.

35. The method according to claim 27, wherein the genetic construct is delivered to the CSF by injection between lumbar vertebrae L3/L4, L4/L5 or L5/S1, wherein the method further comprises injecting a contrast media in combination with the genetic construct.

36. The method according to claim 27, wherein the neurodegenerative disorder to be treated is a disease associated with catecholamine dysfunction.

37. The method according to claim 27, wherein the neurodegenerative disorder to be treated is selected from the group consisting of Parkinson's disease, DOPA responsive dystonia, vascular Parkinsonism, side effects associated with L-DOPA treatment, or L-DOPA induced dyskinesia.

38. The method according to claim 27, wherein the neurodegenerative disorder to be treated is Parkinson's disease.

39. The method according to claim 27, wherein the first coding sequence comprises a nucleotide sequence substantially as set out in SEQ ID NO: 1 or SEQ ID No:2, or a fragment or variant thereof, and/or comprises a nucleotide sequence encoding an amino acid sequence substantially as set out in SEQ ID NO: 21 or SEQ ID No:22, or a fragment or variant thereof.

40. The method according to claim 27, wherein the second coding sequence comprises a nucleotide sequence substantially as set out in SEQ ID NO: 4, or a fragment or variant thereof, and/or comprises a nucleotide sequence encoding an amino acid sequence substantially as set out in SEQ ID NO: 23, or a fragment or variant thereof.

41. The method according to claim 27, wherein the construct further comprises a third coding sequence, which encodes 6-pyruvoyltetrahydropterin (PTPS), wherein the third coding sequence is 3′ to the second coding sequence and is part of the a single operon.

42. The method according to claim 41, wherein the third coding sequence comprises a nucleotide sequence substantially as set out in SEQ ID NO: 32, or a fragment or variant thereof, and/or comprises a nucleotide sequence encoding an amino acid sequence substantially as set out in SEQ ID NO: 33, or a fragment or variant thereof.

43. The method according to claim 27, wherein the construct comprises a sequence substantially as set out in SEQ ID NO: 18, SEQ ID NO: 19, or SEQ ID NO: 20, or a fragment or variant thereof.

44. A method of treating, preventing, or ameliorating a neurodegenerative disorder in a subject, wherein the method comprises administering, to a subject in need of such treatment, a recombinant vector comprising the genetic construct according to claim 27, wherein the vector is delivered to the cerebrospinal fluid (CSF) of the subject.

45. The method according to claim 44, wherein the recombinant vector is a recombinant AAV vector, or wherein the vector does not comprise a modified capsid.

46. A method of treating, preventing, or ameliorating a neurodegenerative disorder in a subject, wherein the method comprises administering, to a subject in need of such treatment, a pharmaceutical composition comprising the genetic construct defined in claim 27 and a pharmaceutically acceptable vehicle, wherein the pharmaceutical composition is delivered to the cerebrospinal fluid (CSF) of a subject.

47. A method of treating, preventing, or ameliorating a neurodegenerative disorder in a subject, wherein the method comprises administering, to a subject in need of such treatment, a pharmaceutical composition comprising the recombinant vector defined in claim 44 and a pharmaceutically acceptable vehicle, wherein the pharmaceutical composition is delivered to the cerebrospinal fluid (CSF) of a subject.
